SEQUENCE LISTING (239 sequences)

SEQ ID NO: 1 - protein, 20 aa, homo sapiens
  MUC1 hypoglycosylated epitope region
HGVTSAPDTRPAPGSTAPPA

SEQ ID NO: 2 - protein, 21 aa, homo sapiens
  CD8&#945; signal peptide
MALPVTALLLPLALLLHAARP

SEQ ID NO: 3 - protein, 40 aa, homo sapiens
  R2 suicide switch
SDPGSGGGGSCPYSNPSLCSGGGGSCPYSNPSLCSGGGGS

SEQ ID NO: 4 - protein, 45 aa, homo sapiens
  CD8&#945; hinge
TTTPAPRPPTPAPTIASQPLSLRPEACRPAAGGAVHTRGLDFACD

SEQ ID NO: 5 - protein, 24 aa, homo sapiens
  CD8&#945; transmembrane domain
IYIWAPLAGTCGVLLLSLVITLYC

SEQ ID NO: 6 - protein, 42 aa, homo sapiens
  4-1BB co-stimulatory domain
KRGRKKLLYIFKQPFMRPVQTTQEEDGCSCRFPEEEEGGCEL

SEQ ID NO: 7 - protein, 112 aa, homo sapiens
  CD3&#950; signalling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 15 aa, artifical
  Linker
GGGGSGGGGSGGGGS

SEQ ID NO: 9 - protein, 118 aa, artifical
  heavy chain variable region MUC1-A
QVQLQQSGAELARPGASVKLSCKASGYTFTNYGLSWVKQRTGQGLEWIGENHPGSGIIYHNEKFRGKATLTADKSSSTAYVQLSSLTSEDSAVYFCARSSGTRGFAYWGQGTLVTVSA

SEQ ID NO: 10 - protein, 114 aa, artifical
  light chain variable region MUC1-A
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHGPWTFGGGTKLEIKRA

SEQ ID NO: 11 - protein, 5 aa, artifical
  CDR1VH-A
NYGLS

SEQ ID NO: 12 - protein, 17 aa, artifical
  CDR2VH-A
ENHPGSGIIYHNEKFRG

SEQ ID NO: 13 - protein, 9 aa, artifical
  CDR3VH-A
SSGTRGFAY

SEQ ID NO: 14 - protein, 16 aa, artifical
  CDR1VL-A
RSSQSIVHSNGNTYLE

SEQ ID NO: 15 - protein, 7 aa, artifical
  CDR2VL-A
KVSNRFS

SEQ ID NO: 16 - protein, 9 aa, artifical
  CDR3VL-A
FQGSHGPWT

SEQ ID NO: 17 - protein, 247 aa, artifical
  MUC1-A full Binding domain
QVQLQQSGAELARPGASVKLSCKASGYTFTNYGLSWVKQRTGQGLEWIGENHPGSGIIYHNEKFRGKATLTADKSSSTAYVQLSSLTSEDSAVYFCARSSGTRGFAYWGQGTLVTVSAGGGGSGGGGSGGGGSDVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHGPWTFGGGTKLEIKRA

SEQ ID NO: 18 - protein, 528 aa, artifical
  CLS MUC1-A CAR full sequence
MALPVTALLLPLALLLHAARPQVQLQQSGAELARPGASVKLSCKASGYTFTNYGLSWVKQRTGQGLEWIGENHPGSGIIYHNEKFRGKATLTADKSSSTAYVQLSSLTSEDSAVYFCARSSGTRGFAYWGQGTLVTVSAGGGGSGGGGSGGGGSDVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHGPWTFGGGTKLEIKRAGSGGGGSCPYSNPSLCSGGGGSCPYSNPSLCSGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 117 aa, artifical
  heavy chain variable region MUC1-B
EVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSS

SEQ ID NO: 20 - protein, 109 aa, artifical
  light chain variable region MUC1-B
DIVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVL

SEQ ID NO: 21 - protein, 10 aa, artifical
  CDR1VH-B
GFTFSNYWMN

SEQ ID NO: 22 - protein, 19 aa, artifical
  CDR2VH-B
EIRLKSNNYATHYAESVKG

SEQ ID NO: 23 - protein, 6 aa, artifical
  CDR3VH-B
GNSFAY

SEQ ID NO: 24 - protein, 11 aa, artifical
  CDR1VL-B
TGAVTTSNYAN

SEQ ID NO: 25 - protein, 8 aa, artifical
  CDR2VL-B
GGTNNRAP

SEQ ID NO: 26 - protein, 9 aa, artifical
  CDR3VL-B
ALWYSNHWV

SEQ ID NO: 27 - protein, 241 aa, artifical
  MUC1-B full Binding domain
EVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGGGSGGGGSGGGGSDIVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVL

SEQ ID NO: 28 - protein, 522 aa, artifical
  CLS MUC1-B CAR  - Full sequence
MALPVTALLLPLALLLHAARPEVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGGGSGGGGSGGGGSDIVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSGGGGSCPYSNPSLCSGGGGSCPYSNPSLCSGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 120 aa, artifical
  heavy chain variable region MUC1-C
QMQLVQSEAELKKPGASVKVSCKASGYSFTGHYMHWVRQAPGQGLEWMGWIDPVTGGTKYAQNFQGWVTMTRDTSIRTAYLELSRLRSDDTAMYYCAREVTGDRGQFDKWGQGTLVTVAS

SEQ ID NO: 30 - protein, 107 aa, artifical
  light chain variable region MUC1-C
QSVLTQPPSVSVAPGKTARITCGGNNIGSKSVHWYQQKPGQAPALVIYYGSNRPSGIPERFSGSNSGNTATLTISRVEAGDEADYYCQVWDSSSDWVFGGGTKLTVL

SEQ ID NO: 31 - protein, 8 aa, artifical
  CDR1VH-C
GYSFTGHY

SEQ ID NO: 32 - protein, 8 aa, artifical
  CDR2VH-C
IDPVTGGT

SEQ ID NO: 33 - protein, 10 aa, artifical
  CDR3VH-C
AREVTGDRGQ

SEQ ID NO: 34 - protein, 6 aa, artifical
  CDR1VL-C
NIGSKS

SEQ ID NO: 35 - protein, 3 aa, artifical
  CDR2VL-C
YGS

SEQ ID NO: 36 - protein, 10 aa, artifical
  CDR3VL-C
QVWDSSSDWV

SEQ ID NO: 37 - protein, 242 aa, artifical
  MUC1-C full Binding domain
QMQLVQSEAELKKPGASVKVSCKASGYSFTGHYMHWVRQAPGQGLEWMGWIDPVTGGTKYAQNFQGWVTMTRDTSIRTAYLELSRLRSDDTAMYYCAREVTGDRGQFDKWGQGTLVTVASGGGGSGGGGSGGGGSQSVLTQPPSVSVAPGKTARITCGGNNIGSKSVHWYQQKPGQAPALVIYYGSNRPSGIPERFSGSNSGNTATLTISRVEAGDEADYYCQVWDSSSDWVFGGGTKLTVL

SEQ ID NO: 38 - protein, 523 aa, artifical
  CLS MUC1-C CAR - Full sequence
MALPVTALLLPLALLLHAARPQMQLVQSEAELKKPGASVKVSCKASGYSFTGHYMHWVRQAPGQGLEWMGWIDPVTGGTKYAQNFQGWVTMTRDTSIRTAYLELSRLRSDDTAMYYCAREVTGDRGQFDKWGQGTLVTVASGGGGSGGGGSGGGGSQSVLTQPPSVSVAPGKTARITCGGNNIGSKSVHWYQQKPGQAPALVIYYGSNRPSGIPERFSGSNSGNTATLTISRVEAGDEADYYCQVWDSSSDWVFGGGTKLTVLGSGGGGSCPYSNPSLCSGGGGSCPYSNPSLCSGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - protein, 116 aa, artifical
  heavy chain variable region MUC1-D
QVQLQQSDAELVKPGSSVKISCKASGYTFTDHAIHWVKQKPEQGLEWIGHFSPGNTDIKYNDKFKGKATLTVDRSSSTAYMQLNSLTSEDSAVYFCKTSTFFFDYWGQGTTLTVSS

SEQ ID NO: 40 - protein, 113 aa, artifical
  light chain variable region MUC1-D
DIVMTQSPSSLTVTAGEKVTMICKSSQSLLNSGDQKNYLTWYQQKPGQPPKLLIFWASTRESGVPDRFTGSGSGTDFTLTISSVQAEDLAVYYCQNDYSYPLTFGAGTKLELK

SEQ ID NO: 41 - protein, 9 aa, artifical
  CDR1VH-D
YTFTDHAIH

SEQ ID NO: 42 - protein, 14 aa, artifical
  CDR2VH-D
WIGHFSPGNTDIKY

SEQ ID NO: 43 - protein, 9 aa, artifical
  CDR3VH-D
KTSTFFFDY

SEQ ID NO: 44 - protein, 14 aa, artifical
  CDR1VL-D
QSLLNSGDQKNYLT

SEQ ID NO: 45 - protein, 11 aa, artifical
  CDR2VL-D
LLIFWASTRES

SEQ ID NO: 46 - protein, 8 aa, artifical
  CDR3VL-D
QNDYSYPL

SEQ ID NO: 47 - protein, 244 aa, artifical
  MUC1-D full Binding domain
QVQLQQSDAELVKPGSSVKISCKASGYTFTDHAIHWVKQKPEQGLEWIGHFSPGNTDIKYNDKFKGKATLTVDRSSSTAYMQLNSLTSEDSAVYFCKTSTFFFDYWGQGTTLTVSSGGGGSGGGGSGGGGSDIVMTQSPSSLTVTAGEKVTMICKSSQSLLNSGDQKNYLTWYQQKPGQPPKLLIFWASTRESGVPDRFTGSGSGTDFTLTISSVQAEDLAVYYCQNDYSYPLTFGAGTKLELK

SEQ ID NO: 48 - protein, 525 aa, artifical
  CLS MUC1-D CAR - Full sequence
MALPVTALLLPLALLLHAARPQVQLQQSDAELVKPGSSVKISCKASGYTFTDHAIHWVKQKPEQGLEWIGHFSPGNTDIKYNDKFKGKATLTVDRSSSTAYMQLNSLTSEDSAVYFCKTSTFFFDYWGQGTTLTVSSGGGGSGGGGSGGGGSDIVMTQSPSSLTVTAGEKVTMICKSSQSLLNSGDQKNYLTWYQQKPGQPPKLLIFWASTRESGVPDRFTGSGSGTDFTLTISSVQAEDLAVYYCQNDYSYPLTFGAGTKLELKGSGGGGSCPYSNPSLCSGGGGSCPYSNPSLCSGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - protein, 9 aa, artifical
  Mimotope Rituximab
CPYSNPSLC

SEQ ID NO: 50 - protein, 24 aa, artifical
  Epitope Palivizumab
NSELLSLINDMPITNDQKKLMSNN

SEQ ID NO: 51 - protein, 12 aa, artifical
  Mimotope 1 Cetuximab
CQFDLSTRRLKC

SEQ ID NO: 52 - protein, 12 aa, artifical
  Mimotope 2 Cetuximab
CQYNLSSRALKC

SEQ ID NO: 53 - protein, 12 aa, artifical
  Mimotope 3 Cetuximab
CVWQRWQKSYVC

SEQ ID NO: 54 - protein, 12 aa, artifical
  Mimotope 4 Cetuximab
CMWDRFSRWYKC

SEQ ID NO: 55 - protein, 25 aa, artifical
  Epitope 1 Nivolumab
SFVLNWYRMSPSNQTDKLAAFPEDR

SEQ ID NO: 56 - protein, 19 aa, artifical
  Epitope 2 Nivolumab
SGTYLCGAISLAPKAQIKE

SEQ ID NO: 57 - protein, 24 aa, artifical
  QBEND-10 Epitope
ELPTQGTFSNVSTNVSPAKPTTTA

SEQ ID NO: 58 - protein, 12 aa, artifical
  Alemtuzumab Epitope
GQNDTSQTSSPS

SEQ ID NO: 59 - protein, 299 aa, artifical
  dnTGFbetaR2
VLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSS

SEQ ID NO: 60 - protein, 335 aa, artifical
  HLA-E
HSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYYKAEWSDSAQGSESHSL

SEQ ID NO: 61 - protein, 603 aa, artifical
  HLA-G
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGATNFSLLKQAGDVEENPGPMCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 62 - protein, 603 aa, artifical; identical to SEQ ID NO: 61
  IL-12a-P2A- IL-12b

SEQ ID NO: 63 - protein, 253 aa, homo sapiens
  IL-12a
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 64 - protein, 328 aa, homo sapiens
  IL-12b
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 65 - protein, 378 aa, artifical
  IgEss_IL-15a-P2A-IgEss_IL-15RA
GTGSATMDWTWILFLVAAATRVHSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGATNFSLLKQAGDVEENPGPGTGSATMDWTWILFLVAAATRVHSITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTT

SEQ ID NO: 66 - protein, 133 aa, homo sapiens
  IL-15a
GIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 67 - protein, 175 aa, homo sapiens
  IL-15RA
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTT

SEQ ID NO: 68 - protein, 182 aa, homo sapiens
  IL-18
MGTGSATMDWTWILFLVAAATRVHSYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 69 - protein, 405 aa, homo sapiens
  HYAL1
MAAHLLPICALFLTLLDMAQGFRGPLLPNRPFTTVWNANTQWCLERHGVDVDVSVFDVVANPGQTFRGPDMTIFYSSQLGTYPYYTPTGEPVFGGLPQNASLIAHLARTFQDILAAIPAPDFSGLAVIDWEAWRPRWAFNWDTKDIYRQRSRALVQAQHPDWPAPQVEAVAQDQFQGAARAWMAGTLQLGRALRPRGLWGFYGFPDCYNYDFLSPNYTGQCPSGIRAQNDQLGWLWGQSRALYPSIYMPAVLEGTGKSQMYVQHRVAEAFRVAVAAGDPNLPVLPYVQIFYDTTNHFLPLESCQAIKEYMDTTLGPFILNVTSGALLCSQALCSGHGRCVRRTSHPKALLLLNPASFSIQLTPGGGPLSLRGALSLEDQAQMAVEFKCRCYPGWQAPWCERKSMW

SEQ ID NO: 70 - protein, 473 aa, homo sapiens
  HYAL2
MRAGPGPTVTLALVLAVSWAMELKPTAPPIFTGRPFVVAWDVPTQDCGPRLKVPLDLNAFDVQASPNEGFVNQNITIFYRDRLGLYPRFDSAGRSVHGGVPQNVSLWAHRKMLQKRVEHYIRTQESAGLAVIDWEDWRPVWVRNWQDKDVYRRLSRQLVASRHPDWPPDRIVKQAQYEFEFAAQQFMLETLRYVKAVRPRHLWGFYLFPDCYNHDYVQNWESYTGRCPDVEVARNDQLAWLWAESTALFPSVYLDETLASSRHGRNFVSFRVQEALRVARTHHANHALPVYVFTRPTYSRRLTGLSEMDLISTIGESAALGAAGVILWGDAGYTTSTETCQYLKDYLTRLLVPYVVNVSWATQYCSRAQCHGHGRCVRRNPSASTFLHLSTNSFRLVPGHAPGEPQLRPVGELSWADIDHLQTHFRCQCYLGWSGEQCQWDHRQAAGGASEAWAGSHLTSLLALAALAFTWTL

SEQ ID NO: 71 - protein, 511 aa, homo sapiens
  SPAM1
MGVLKFKHIFFRSFVKSSGVSQIVFTFLLIPCCLTLNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIWRLEVWDQGISRIGFF

SEQ ID NO: 72 - protein, 569 aa, homo sapiens
  GPI1
MVALCSLQHLGSSDPRALPTLPTATSGQRPAKRRRKSPAMAALTRDPQFQKLQQWYREHRSELNLRRLFDANKDRFNHFSLTLNTNHGHILVDYSKNLVTEDVMRMLVDLAKSRGVEAARERMFNGEKINYTEGRAVLHVALRNRSNTPILVDGKDVMPEVNKVLDKMKSFCQGPLMVTEALKPYSSGGPRVWYVSNIDGTHIAKTLAQLNPESSLFIIASKTFTTQETITNAETAKEWFLQAAKDPSAVAKHFVALSTNTTKVKEFGIDPQNMFEFWDWVGGRYSLWSAIGLSIALHVGFDNFEQLLSGAHWMDQHFRTTPLEKNAPVLLALLGIWYINCFGCETHAMLPYDQYLHRFAAYFQQGDMESNGKYITKSGTRVDHQTGPIVWGEPGTNGQHAFYQLIHQGTKMIPCDFLIPVQTQHPIRKGLHHKILLANFLAQTEALMRGKSTEEARKELQAAGKSPEDLERLLPHKVFEGNRPTNSIVFTKLTPFMLGALVAMYEHKIFVQGIIWDINSFDQWGVELGKQLAKKIEPELDGSAQVTSHDASTNGLINFIKQQREARVQ

SEQ ID NO: 73 - protein, 622 aa, homo sapiens
  PCK1
MPPQLQNGLNLSAKVVQGSLDSLPQAVREFLENNAELCQPDHIHICDGSEEENGRLLGQMEEEGILRRLKKYDNCWLALTDPRDVARIESKTVIVTQEQRDTVPIPKTGLSQLGRWMSEEDFEKAFNARFPGCMKGRTMYVIPFSMGPLGSPLSKIGIELTDSPYVVASMRIMTRMGTPVLEAVGDGEFVKCLHSVGCPLPLQKPLVNNWPCNPELTLIAHLPDRREIISFGSGYGGNSLLGKKCFALRMASRLAKEEGWLAEHMLILGITNPEGEKKYLAAAFPSACGKTNLAMMNPSLPGWKVECVGDDIAWMKFDAQGHLRAINPENGFFGVAPGTSVKTNPNAIKTIQKNTIFTNVAETSDGGVYWEGIDEPLASGVTITSWKNKEWSSEDGEPCAHPNSRFCTPASQCPIIDAAWESPEGVPIEGIIFGGRRPAGVPLVYEALSWQHGVFVGAAMRSEATAAAEHKGKIIMHDPFAMRPFFGYNFGKYLAHWLSMAQHPAAKLPKIFHVNWFRKDKEGKFLWPGFGENSRVLEWMFNRIDGKASTKLTPIGYIPKEDALNLKGLGHINMMELFSISKEFWEKEVEDIEKYLEDQVNADLPCEIEREILALKQRISQM

SEQ ID NO: 74 - protein, 332 aa, homo sapiens
  LDHA
MATLKDQLIYNLLKEEQTPQNKITVVGVGAVGMACAISILMKDLADELALVDVIEDKLKGEMMDLQHGSLFLRTPKIVSGKDYNVTANSKLVIITAGARQQEGESRLNLVQRNVNIFKFIIPNVVKYSPNCKLLIVSNPVDILTYVAWKISGFPKNRVIGSGCNLDSARFRYLMGERLGVHPLSCHGWVLGEHGDSSVPVWSGMNVAGVSLKTLHPDLGTDKDKEQWKEVHKQVVESAYEVIKLKGYTSWAIGLSVADLAESIMKNLRRVHPVSTMIKGLYGIKDDVFLSVPCILGQNGISDLVKVTLTSEEEARLKKSADTLWGIQKELQF

SEQ ID NO: 75 - protein, 925 aa, artificial
  TRAC TALEN Left
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 76 - protein, 925 aa, artificial
  TRAC TALEN Right
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNNGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 77 - protein, 925 aa, artificial
  PD1 TALEN Left
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASKLGGKQALETVQALLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASYKGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 78 - protein, 924 aa, artificial
  PD1 TALEN Right
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 79 - protein, 925 aa, artificial
  B2M TALEN Left
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 80 - protein, 925 aa, artificial
  B2M TALEN Right
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 81 - DNA, 59 bp, homo sapiens
  TRAC target sequence
tgatcctcttgtcccacagatatccagaaccctgaccctgccgtgtaccagctgagaga

SEQ ID NO: 82 - DNA, 49 bp, homo sapiens
  PD1 target sequence
tacctctgtggggccatctccctggcccccaaggcgcagatcaaagaga

SEQ ID NO: 83 - DNA, 47 bp, homo sapiens
  B2M target sequence
ttagctgtgctcgcgctactctctctttctggcctggaggctatcca

SEQ ID NO: 84 - DNA, 3639 bp, artificial
  AAV6 PD1KO IL-12 (from pCLS30511)
gactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccggttctggcgtgaaacagactttgaattttgaccttctcaagttggcgggagacgtggagtccaacccagggcccatgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttccggaagcggagctactaacttcagcctgctgaagcaggctggagacgtggaggagaaccctggacctatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtgagggcagaggcagcctgctgacctgcggcgacgtcgaggagaaccccgggcccatgggggcaggtgccaccggccgcgccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgatgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtgatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgactagtggcgaattcggcgcagatcaaagagagcctgcgggcagagctcagggtgacaggtgcggcctcggaggccccggggcaggggtgagctgagccggtcctggggtgggtgtcccctcctgcacaggatcaggagctccagggtcgtagggcagggaccccccagctccagtccagggctctgtcctgcacctggggaatggtgaccggcatctctgtcctctagctctggaagcaccccagcccctctagtctgccctcacccctgaccctgaccctccaccctgaccccgtcctaacccctgacctttg

SEQ ID NO: 85 - DNA, 2289 bp, artificial
  AAV6 B2MKO HLA-E (from pCLS31913)
cgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagatgtctcgctccgtggccttagctgtgctcgcgctactctctcttagcggcctcgaagctgttatggctccgcggactttaattttaggtggtggcggatccggtggtggcggttctggtggtggcggctccatccagcgtacgcccaaaattcaagtctacagccgacatcctgcagagaacggcaaatctaatttcctgaactgctatgtatcaggctttcaccctagcgatatagaagtggacctgctgaaaaacggagagaggatagaaaaggtcgaacacagcgacctctccttttccaaggactggagcttttatcttctgtattatactgaatttacacccacggaaaaagatgagtatgcgtgccgagtaaaccacgtcacgctgtcacagcccaaaatagtaaaatgggatcgcgacatgggtggtggcggttctggtggtggcggtagtggcggcggaggaagcggtggtggcggttccggatctcactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaacctgcggacgctgcgcggctactacaatcagagcgaggccgggtctcacaccctgcagtggatgcatggctgcgagctggggcccgacaggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggctctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactataaggctgagtggagcgacagtgcccaggggtctgagtctcacagcttgtaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgtctctttctggcctggaggctatccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaag

SEQ ID NO: 86 - DNA, 49 bp, artificial
  Target Sequence into TGFbetaRII T003387
ttttgtttccccatcagaatataacaccagcaatcctgacttgttgcta

SEQ ID NO: 87 - DNA, 49 bp, artificial
  Target Sequence into TGFbetaRII T003401
tccctatgaggagtatgcctcttggaagacagagaaggacatcttctca

SEQ ID NO: 88 - DNA, 49 bp, artificial
  Target Sequence into TGFbetaRII T003400
tggcagtcaagatctttccctatgaggagtatgcctcttggaagacaga

SEQ ID NO: 89 - DNA, 49 bp, artificial
  Target Sequence into TGFbetaRII T003405
tgtgggaggcccaagatgcccatcgtgcacagggacctcaagagctcca

SEQ ID NO: 90 - DNA, 23 bp, artificial
  sgRNA sequence targetting TGFbetaRII gene
acagtgatcacactccatgtggg

SEQ ID NO: 91 - DNA, 23 bp, artificial
  sgRNA sequence targetting TGFbetaRII gene
gcagaagctgagttcaacctggg

SEQ ID NO: 92 - DNA, 23 bp, artificial
  sgRNA sequence targetting TGFbetaRII gene
aggttaggtcgttcttcacgagg

SEQ ID NO: 93 - DNA, 23 bp, artificial
  sgRNA sequence targetting TGFbetaRII gene
aaagcgacctttccccaccaggg

SEQ ID NO: 94 - DNA, 23 bp, artificial
  sgRNA sequence targetting TGFbetaRII gene
tggatgacctggctaacagtggg

SEQ ID NO: 95 - DNA, 23 bp, artificial
  sgRNA sequence targetting TGFbetaRII gene
cctgggaaaccggcaagacgcgg

SEQ ID NO: 96 - DNA, 23 bp, artificial
  sgRNA sequence targetting TGFbetaRII gene
acagatatggcaactcccagtgg

SEQ ID NO: 97 - DNA, 23 bp, artificial
  sgRNA sequence targetting TGFbetaRII gene
gtggaggtgagcaatcccccggg

SEQ ID NO: 98 - DNA, 23 bp, artificial
  sgRNA sequence targetting TGFbetaRII gene
acctacaggagtacctgacgcgg

SEQ ID NO: 99 - DNA, 23 bp, artificial
  sgRNA sequence targetting TGFbetaRII gene
gtgatcacactccatgtgggagg

SEQ ID NO: 100 - DNA, 23 bp, artificial
  sgRNA sequence targetting TGFbetaRII gene
gctggtgttatattctgatgggg

SEQ ID NO: 101 - DNA, 23 bp, artificial
  sgRNA sequence targetting TGFbetaRII gene
cacagtgatcacactccatgtgg

SEQ ID NO: 102 - DNA, 23 bp, artificial
  sgRNA sequence targetting TGFbetaRII gene
cacatggagtgtgatcactgtgg

SEQ ID NO: 103 - DNA, 23 bp, artificial
  sgRNA sequence targetting TGFbetaRII gene
cagagtagggtccagacgcaggg

SEQ ID NO: 104 - DNA, 23 bp, artificial
  sgRNA sequence targetting TGFbetaRII gene
gcttctgctgccggttaacgcgg

SEQ ID NO: 105 - DNA, 23 bp, artificial
  sgRNA sequence targetting TGFbetaRII gene
gtggatgacctggctaacagtgg

SEQ ID NO: 106 - DNA, 23 bp, artificial
  sgRNA sequence targetting TGFbetaRII gene
gggaaagcccaaagtcacacagg

SEQ ID NO: 107 - DNA, 23 bp, artificial
  sgRNA sequence targetting TGFbetaRII gene
aatatgactagcaacaagtcagg

SEQ ID NO: 108 - DNA, 23 bp, artificial
  sgRNA sequence targetting TGFbetaRII gene
atggagtgtgatcactgtggagg

SEQ ID NO: 109 - DNA, 23 bp, artificial
  sgRNA sequence targetting TGFbetaRII gene
atcaccgccttccacgccaaggg

SEQ ID NO: 110 - DNA, 23 bp, artificial
  sgRNA sequence targetting TGFbetaRII gene
aggagcggaagacggagttgggg

SEQ ID NO: 111 - DNA, 23 bp, artificial
  sgRNA sequence targetting TGFbetaRII gene
ccacgccaagggcaacctacagg

SEQ ID NO: 112 - DNA, 23 bp, artificial
  sgRNA sequence targetting TGFbetaRII gene
caagatgcccatcgtgcacaggg

SEQ ID NO: 113 - DNA, 23 bp, artificial
  sgRNA sequence targetting TGFbetaRII gene
gatatggcaactcccagtggtgg

SEQ ID NO: 114 - DNA, 23 bp, artificial
  sgRNA sequence targetting TGFbetaRII gene
agcagaagctgagttcaacctgg

SEQ ID NO: 115 - DNA, 23 bp, artificial
  sgRNA sequence targetting TGFbetaRII gene
cctgtaggttgcccttggcgtgg

SEQ ID NO: 116 - DNA, 23 bp, artificial
  sgRNA sequence targetting TGFbetaRII gene
gtgagcaatcccccgggcgaggg

SEQ ID NO: 117 - DNA, 23 bp, artificial
  sgRNA sequence targetting TGFbetaRII gene
acagagtagggtccagacgcagg

SEQ ID NO: 118 - DNA, 23 bp, artificial
  sgRNA sequence targetting TGFbetaRII gene
gacgcggcatgtcatcagctggg

SEQ ID NO: 119 - DNA, 23 bp, artificial
  sgRNA sequence targetting TGFbetaRII gene
ggtcatccacagacagagtaggg

SEQ ID NO: 120 - DNA, 23 bp, artificial
  sgRNA sequence targetting TGFbetaRII gene
agtcaagatctttccctatgagg

SEQ ID NO: 121 - DNA, 23 bp, artificial
  sgRNA sequence targetting TGFbetaRII gene
gaacatactccagttcctgacgg

SEQ ID NO: 122 - DNA, 23 bp, artificial
  sgRNA sequence targetting TGFbetaRII gene
acgtggagctgatgtcagagcgg

SEQ ID NO: 123 - DNA, 23 bp, artificial
  sgRNA sequence targetting TGFbetaRII gene
ggggaaaggtcgctttgctgagg

SEQ ID NO: 124 - DNA, 23 bp, artificial
  sgRNA sequence targetting TGFbetaRII gene
tctggaccctactctgtctgtgg

SEQ ID NO: 125 - DNA, 23 bp, artificial
  sgRNA sequence targetting TGFbetaRII gene
tgggcagctccctcgcccggggg

SEQ ID NO: 126 - DNA, 23 bp, artificial
  sgRNA sequence targetting TGFbetaRII gene
agctgatgacatgccgcgtcagg

SEQ ID NO: 127 - DNA, 23 bp, artificial
  sgRNA sequence targetting TGFbetaRII gene
gccgcgtcaggtactcctgtagg

SEQ ID NO: 128 - DNA, 23 bp, artificial
  sgRNA sequence targetting TGFbetaRII gene
caagaggcatactcctcataggg

SEQ ID NO: 129 - DNA, 23 bp, artificial
  sgRNA sequence targetting TGFbetaRII gene
ggtgagcaatcccccgggcgagg

SEQ ID NO: 130 - DNA, 23 bp, artificial
  sgRNA sequence targetting TGFbetaRII gene
ttgctggtgttatattctgatgg

SEQ ID NO: 131 - DNA, 23 bp, artificial
  sgRNA sequence targetting TGFbetaRII gene
cctatgaggagtatgcctcttgg

SEQ ID NO: 132 - DNA, 23 bp, artificial
  sgRNA sequence targetting TGFbetaRII gene
tgctggtgttatattctgatggg

SEQ ID NO: 133 - DNA, 23 bp, artificial
  sgRNA sequence targetting TGFbetaRII gene
cgaggatattggagctcttgagg

SEQ ID NO: 134 - DNA, 23 bp, artificial
  sgRNA sequence targetting TGFbetaRII gene
gttgatgttgttggcacacgtgg

SEQ ID NO: 135 - DNA, 23 bp, artificial
  sgRNA sequence targetting TGFbetaRII gene
tgacgcggcatgtcatcagctgg

SEQ ID NO: 136 - DNA, 23 bp, artificial
  sgRNA sequence targetting TGFbetaRII gene
ttgagctggacaccctggtgggg

SEQ ID NO: 137 - DNA, 23 bp, artificial
  sgRNA sequence targetting TGFbetaRII gene
ttcagagcagtttgagacagtgg

SEQ ID NO: 138 - DNA, 23 bp, artificial
  sgRNA sequence targetting TGFbetaRII gene
gcggcatgtcatcagctgggagg

SEQ ID NO: 139 - DNA, 23 bp, artificial
  sgRNA sequence targetting TGFbetaRII gene
aggtcatccacagacagagtagg

SEQ ID NO: 140 - DNA, 23 bp, artificial
  sgRNA sequence targetting TGFbetaRII gene
gaagatgatgatgacagatatgg

SEQ ID NO: 141 - DNA, 23 bp, artificial
  sgRNA sequence targetting TGFbetaRII gene
tgacctggctaacagtgggcagg

SEQ ID NO: 142 - DNA, 23 bp, artificial
  sgRNA sequence targetting TGFbetaRII gene
tctactgctaccgcgttaaccgg

SEQ ID NO: 143 - DNA, 23 bp, artificial
  sgRNA sequence targetting TGFbetaRII gene
gtccttctctgtcttccaagagg

SEQ ID NO: 144 - DNA, 23 bp, artificial
  sgRNA sequence targetting TGFbetaRII gene
attgagctggacaccctggtggg

SEQ ID NO: 145 - DNA, 23 bp, artificial
  sgRNA sequence targetting TGFbetaRII gene
gtcgttcttcacgaggatattgg

SEQ ID NO: 146 - DNA, 23 bp, artificial
  sgRNA sequence targetting TGFbetaRII gene
catcagcctcctgccaccactgg

SEQ ID NO: 147 - DNA, 23 bp, artificial
  sgRNA sequence targetting TGFbetaRII gene
agttcctgacggctgaggagcgg

SEQ ID NO: 148 - DNA, 23 bp, artificial
  sgRNA sequence targetting TGFbetaRII gene
actccagttcctgacggctgagg

SEQ ID NO: 149 - DNA, 23 bp, artificial
  sgRNA sequence targetting TGFbetaRII gene
cttgaggtccctgtgcacgatgg

SEQ ID NO: 150 - DNA, 23 bp, artificial
  sgRNA sequence targetting TGFbetaRII gene
ttgaggtccctgtgcacgatggg

SEQ ID NO: 151 - DNA, 23 bp, artificial
  sgRNA sequence targetting TGFbetaRII gene
ccgcgtcttgccggtttcccagg

SEQ ID NO: 152 - DNA, 23 bp, artificial
  sgRNA sequence targetting TGFbetaRII gene
gttgcccttggcgtggaaggcgg

SEQ ID NO: 153 - DNA, 23 bp, artificial
  sgRNA sequence targetting TGFbetaRII gene
ctttgggctttccctgcgtctgg

SEQ ID NO: 154 - DNA, 23 bp, artificial
  sgRNA sequence targetting TGFbetaRII gene
gatcaccgccttccacgccaagg

SEQ ID NO: 155 - DNA, 23 bp, artificial
  sgRNA sequence targetting TGFbetaRII gene
tgaagtgttctgcttcagcttgg

SEQ ID NO: 156 - DNA, 23 bp, artificial
  sgRNA sequence targetting TGFbetaRII gene
ctgtgcacgatgggcatcttggg

SEQ ID NO: 157 - DNA, 23 bp, artificial
  sgRNA sequence targetting TGFbetaRII gene
ggcatcttgggcctcccacatgg

SEQ ID NO: 158 - DNA, 23 bp, artificial
  sgRNA sequence targetting TGFbetaRII gene
ttacctgcccactgttagccagg

SEQ ID NO: 159 - DNA, 23 bp, artificial
  sgRNA sequence targetting TGFbetaRII gene
atcagcctcctgccaccactggg

SEQ ID NO: 160 - DNA, 23 bp, artificial
  sgRNA sequence targetting TGFbetaRII gene
tcgctttgctgaggtctataagg

SEQ ID NO: 161 - DNA, 23 bp, artificial
  sgRNA sequence targetting TGFbetaRII gene
agtcacacaggcagcaggttagg

SEQ ID NO: 162 - DNA, 23 bp, artificial
  sgRNA sequence targetting TGFbetaRII gene
gaggagcggaagacggagttggg

SEQ ID NO: 163 - DNA, 23 bp, artificial
  sgRNA sequence targetting TGFbetaRII gene
tgggcagcagctctgtgttgtgg

SEQ ID NO: 164 - DNA, 23 bp, artificial
  sgRNA sequence targetting TGFbetaRII gene
tagcaacaagtcaggattgctgg

SEQ ID NO: 165 - DNA, 23 bp, artificial
  sgRNA sequence targetting TGFbetaRII gene
ccaagatgcccatcgtgcacagg

SEQ ID NO: 166 - DNA, 23 bp, artificial
  sgRNA sequence targetting TGFbetaRII gene
tgtggaggtgagcaatcccccgg

SEQ ID NO: 167 - DNA, 23 bp, artificial
  sgRNA sequence targetting TGFbetaRII gene
tgcctcttggaagacagagaagg

SEQ ID NO: 168 - DNA, 23 bp, artificial
  sgRNA sequence targetting TGFbetaRII gene
gcccattgagctggacaccctgg

SEQ ID NO: 169 - DNA, 23 bp, artificial
  sgRNA sequence targetting TGFbetaRII gene
ctgagttcaacctgggaaaccgg

SEQ ID NO: 170 - DNA, 23 bp, artificial
  sgRNA sequence targetting TGFbetaRII gene
tgggaggacctgcgcaagctggg

SEQ ID NO: 171 - DNA, 23 bp, artificial
  sgRNA sequence targetting TGFbetaRII gene
gtactcctgtaggttgcccttgg

SEQ ID NO: 172 - DNA, 23 bp, artificial
  sgRNA sequence targetting TGFbetaRII gene
tcttccgctcctcagccgtcagg

SEQ ID NO: 173 - DNA, 23 bp, artificial
  sgRNA sequence targetting TGFbetaRII gene
ctgggcagctccctcgcccgggg

SEQ ID NO: 174 - DNA, 23 bp, artificial
  sgRNA sequence targetting TGFbetaRII gene
cattgagctggacaccctggtgg

SEQ ID NO: 175 - DNA, 23 bp, artificial
  sgRNA sequence targetting TGFbetaRII gene
tggcaactcccagtggtggcagg

SEQ ID NO: 176 - DNA, 23 bp, artificial
  sgRNA sequence targetting TGFbetaRII gene
caactcccagtggtggcaggagg

SEQ ID NO: 177 - DNA, 23 bp, artificial
  sgRNA sequence targetting TGFbetaRII gene
cgagcactgtgccatcatcctgg

SEQ ID NO: 178 - DNA, 23 bp, artificial
  sgRNA sequence targetting TGFbetaRII gene
gcggtcatcttccaggatgatgg

SEQ ID NO: 179 - DNA, 23 bp, artificial
  sgRNA sequence targetting TGFbetaRII gene
agagctgctgcccattgagctgg

SEQ ID NO: 180 - DNA, 23 bp, artificial
  sgRNA sequence targetting TGFbetaRII gene
accagggtgtccagctcaatggg

SEQ ID NO: 181 - DNA, 23 bp, artificial
  sgRNA sequence targetting TGFbetaRII gene
ctggacaccctggtggggaaagg

SEQ ID NO: 182 - DNA, 23 bp, artificial
  sgRNA sequence targetting TGFbetaRII gene
caaagcgacctttccccaccagg

SEQ ID NO: 183 - DNA, 23 bp, artificial
  sgRNA sequence targetting TGFbetaRII gene
gacggctgaggagcggaagacgg

SEQ ID NO: 184 - DNA, 23 bp, artificial
  sgRNA sequence targetting TGFbetaRII gene
tactctgtctgtggatgacctgg

SEQ ID NO: 185 - DNA, 23 bp, artificial
  sgRNA sequence targetting TGFbetaRII gene
cggcaagacgcggaagctcatgg

SEQ ID NO: 186 - DNA, 23 bp, artificial
  sgRNA sequence targetting TGFbetaRII gene
cccaaagtcacacaggcagcagg

SEQ ID NO: 187 - DNA, 23 bp, artificial
  sgRNA sequence targetting TGFbetaRII gene
taggttgcccttggcgtggaagg

SEQ ID NO: 188 - DNA, 23 bp, artificial
  sgRNA sequence targetting TGFbetaRII gene
tgaggagcggaagacggagttgg

SEQ ID NO: 189 - DNA, 23 bp, artificial
  sgRNA sequence targetting TGFbetaRII gene
cctgtgcacgatgggcatcttgg

SEQ ID NO: 190 - DNA, 23 bp, artificial
  sgRNA sequence targetting TGFbetaRII gene
gggagctgcccagcttgcgcagg

SEQ ID NO: 191 - DNA, 23 bp, artificial
  sgRNA sequence targetting TGFbetaRII gene
ttgaactcagcttctgctgccgg

SEQ ID NO: 192 - DNA, 23 bp, artificial
  sgRNA sequence targetting TGFbetaRII gene
ccaagaggcatactcctcatagg

SEQ ID NO: 193 - DNA, 23 bp, artificial
  sgRNA sequence targetting TGFbetaRII gene
catgagcttccgcgtcttgccgg

SEQ ID NO: 194 - DNA, 23 bp, artificial
  sgRNA sequence targetting TGFbetaRII gene
cggagttggggaaacaatactgg

SEQ ID NO: 195 - DNA, 23 bp, artificial
  sgRNA sequence targetting TGFbetaRII gene
gctcctcagccgtcaggaactgg

SEQ ID NO: 196 - DNA, 23 bp, artificial
  sgRNA sequence targetting TGFbetaRII gene
caccagggtgtccagctcaatgg

SEQ ID NO: 197 - DNA, 23 bp, artificial
  sgRNA sequence targetting TGFbetaRII gene
ctagtcatatttcaagtgacagg

SEQ ID NO: 198 - DNA, 23 bp, artificial
  sgRNA sequence targetting TGFbetaRII gene
gctgggcagctccctcgcccggg

SEQ ID NO: 199 - DNA, 23 bp, artificial
  sgRNA sequence targetting TGFbetaRII gene
tgtcagagcggtcatcttccagg

SEQ ID NO: 200 - DNA, 23 bp, artificial
  sgRNA sequence targetting TGFbetaRII gene
ctgggaggacctgcgcaagctgg

SEQ ID NO: 201 - DNA, 23 bp, artificial
  sgRNA sequence targetting TGFbetaRII gene
agctgggcagctccctcgcccgg

SEQ ID NO: 202 - DNA, 23 bp, artificial
  sgRNA sequence targetting TGFbetaRII gene
tgtgttgtggttgatgttgttgg

SEQ ID NO: 203 - DNA, 23 bp, artificial
  sgRNA sequence targetting TGFbetaRII gene
cctgctgcctgtgtgactttggg

SEQ ID NO: 204 - DNA, 23 bp, artificial
  sgRNA sequence targetting TGFbetaRII gene
acctgctgcctgtgtgactttgg

SEQ ID NO: 205 - DNA, 1521 bp, artificial
  DNA encoding CAR CLS MUC1-A
caggttcaacttcaacaatcaggtgctgaattggcgagacccggcgcttcagtgaaactttcctgtaaggcatccggttacacttttacaaattatggtctctcctgggtcaagcagcgcaccgggcagggcctcgagtggattggcgaaaatcaccctggaagcggcattatatatcataacgagaaattcagaggtaaggcaacgttgacggcagataagagttcaagtaccgcttatgtccaactcagctcattgacttctgaggacagtgcagtttacttctgcgcacgaagttcaggcacgcgggggtttgcctactgggggcagggaacattggttactgtctcagccggaggaggcggtagcggcggcggaggtagtggagggggtggaagtgatgttctgatgactcagacgcctctctctctgcccgtcagcctgggagatcaggcgagcatatcatgcagaagttcccaaagtattgttcattcaaacggtaacacgtatcttgaatggtatttgcagaaaccggggcaaagtccaaaactgctcatatataaagtctcaaaccgctttagcggtgtaccggataggttttccgggagcgggtcagggacagacttcactctcaagatttcccgagtcgaagcagaagatctcggcgtgtactattgcttccagggttcacacggcccttggactttcggaggaggaactaaactcgaaattaagcgagccggatcaggaggtgggggcagctgcccgtacagtaatccaagtctctgctccggaggcggcggctcatgcccatattctaatccatcactttgcagcggcgggggtggtagcactaccacaccagcacctcgaccaccaactccagcaccaaccattgcatcacagccactgagcctgcgaccagaggcctgtagacctgctgcaggaggagctgtgcatacccgaggactggactttgcctgcgatatctacatttgggctcctctggcaggaacttgtggcgtgctgctgctgtccctggtcatcaccctgtactgcaagcgaggccggaagaaactgctgtatattttcaaacagccctttatgagacctgtgcagactacccaggaggaagacggctgcagctgtaggttccccgaggaagaggaaggcgggtgtgagctgagggtcaagtttagccgctccgcagatgcccctgcttaccagcaggggcagaatcagctgtataacgagctgaatctgggacggagagaggaatacgacgtgctggataaaaggcgcgggagagaccccgaaatgggaggcaagccacgacggaaaaacccccaggagggcctgtacaatgaactgcagaaggacaaaatggcagaggcctatagtgaaatcgggatgaagggagagagaaggcgcggcaaagggcacgatggcctgtaccaggggctgtctactgccaccaaggacacctatgatgctctgcatatgcaggcactgcctccaagg

SEQ ID NO: 206 - DNA, 1503 bp, artificial
  DNA encoding CAR CLS MUC1-B
gaagtacagcttcagcaatctgggggaggactggttcaacctgggggatcaatgaaactctcttgcgtcgcttcagggttcacattcagcaactattggatgaactgggtgcgacaaagccccgaaaagggtctggaatgggttgctgagatacgactgaagagcaataactatgccacacactacgctgagtccgtcaaggggagattcacaataagccgggacgatagtaaaagcagtgtatatcttcaaatgaacaatctgcgagctgaggacactggaatatattactgtacttttggaaactctttcgcctactggggacaaggaacgacggtaacagtttcttccggaggaggcggtagcggcggcggaggtagtggagggggtggaagtgacatcgtagtaacgcaggagtcagcgttgaccacctcccccggtgagactgtgacactgacctgtcggagtagcactggcgcagtcacgaccagcaattacgcgaactgggttcaagaaaaaccggaccatctcttcactggactcataggggggactaacaatcgggcaccgggggtgcctgcaaggttttctggttctcttatcggagataaggcagcactgacaataactggggcgcaaactgaggatgaggctatttacttttgtgcactctggtactccaaccactgggtcttcgggggtggcacgaaattgaccgtactcggatcaggaggtgggggcagctgcccgtacagtaatccaagtctctgctccggaggcggcggctcatgcccatattctaatccatcactttgcagcggcgggggtggtagcactaccacaccagcacctcgaccaccaactccagcaccaaccattgcatcacagccactgagcctgcgaccagaggcctgtagacctgctgcaggaggagctgtgcatacccgaggactggactttgcctgcgatatctacatttgggctcctctggcaggaacttgtggcgtgctgctgctgtccctggtcatcaccctgtactgcaagcgaggccggaagaaactgctgtatattttcaaacagccctttatgagacctgtgcagactacccaggaggaagacggctgcagctgtaggttccccgaggaagaggaaggcgggtgtgagctgagggtcaagtttagccgctccgcagatgcccctgcttaccagcaggggcagaatcagctgtataacgagctgaatctgggacggagagaggaatacgacgtgctggataaaaggcgcgggagagaccccgaaatgggaggcaagccacgacggaaaaacccccaggagggcctgtacaatgaactgcagaaggacaaaatggcagaggcctatagtgaaatcgggatgaagggagagagaaggcgcggcaaagggcacgatggcctgtaccaggggctgtctactgccaccaaggacacctatgatgctctgcatatgcaggcactgcctccaagg

SEQ ID NO: 207 - DNA, 1506 bp, artificial
  DNA encoding CAR CLS MUC1-C
cagatgcaactggttcaatccgaagcggagttgaagaaacctggcgcatctgtcaaagtatcctgcaaagctagtggttacagcttcacgggtcactatatgcactgggtcagacaagcgccaggccagggactggaatggatggggtggatagacccggtgaccggaggaactaagtatgctcagaacttccaaggatgggtcacaatgacaagggatacgtccatccgaaccgcctaccttgaactcagccgactccggtctgacgatactgcaatgtactattgcgcgagagaagtaaccggggacagggggcaatttgacaaatggggacaggggaccttggtcactgtggccagcggaggaggcggtagcggcggcggaggtagtggagggggtggaagtcagagtgtgctcacccagcctccatccgtgtctgtggcacctggtaaaacggcacgaattacttgtggggggaacaatattggatctaaatctgtccactggtatcagcagaaaccggggcaagctccggcgcttgtcatatactatggatctaaccgaccgagcgggattccagaacggtttagcggctctaatagcggtaatacagcaactcttacgatttcaagggtagaagcgggcgatgaggcagactactactgccaggtatgggatagtagttctgattgggtcttcggcggggggacaaaacttacggttctcggatcaggaggtgggggcagctgcccgtacagtaatccaagtctctgctccggaggcggcggctcatgcccatattctaatccatcactttgcagcggcgggggtggtagcactaccacaccagcacctcgaccaccaactccagcaccaaccattgcatcacagccactgagcctgcgaccagaggcctgtagacctgctgcaggaggagctgtgcatacccgaggactggactttgcctgcgatatctacatttgggctcctctggcaggaacttgtggcgtgctgctgctgtccctggtcatcaccctgtactgcaagcgaggccggaagaaactgctgtatattttcaaacagccctttatgagacctgtgcagactacccaggaggaagacggctgcagctgtaggttccccgaggaagaggaaggcgggtgtgagctgagggtcaagtttagccgctccgcagatgcccctgcttaccagcaggggcagaatcagctgtataacgagctgaatctgggacggagagaggaatacgacgtgctggataaaaggcgcgggagagaccccgaaatgggaggcaagccacgacggaaaaacccccaggagggcctgtacaatgaactgcagaaggacaaaatggcagaggcctatagtgaaatcgggatgaagggagagagaaggcgcggcaaagggcacgatggcctgtaccaggggctgtctactgccaccaaggacacctatgatgctctgcatatgcaggcactgcctccaagg

SEQ ID NO: 208 - DNA, 1512 bp, artificial
  DNA encoding CAR CLS MUC1-D
caggtacagctccagcaatccgacgcggaattggttaaaccggggagctccgtaaaaatttcatgcaaagcgtctggctataccttcacagaccacgctatacattgggtgaagcagaaaccagaacaaggactcgaatggatcggtcacttctcaccgggtaatacggacattaaatacaacgacaaattcaagggaaaggcgacactcacggtagatagatcaagttccacagcttatatgcaactgaatagcctcacttctgaggattccgcagtctatttttgcaaaacaagcacctttttctttgactattggggccaaggcactactttgacagtgtcctctggaggaggcggtagcggcggcggaggtagtggagggggtggaagtgacatcgtaatgactcaatccccgtcaagtcttaccgtgacggcaggcgaaaaggtaaccatgatctgcaagtcatctcaatccctgcttaacagtggtgaccaaaagaattacctgacatggtaccaacaaaagcccggccagccaccaaaactcctcatattttgggcgtcaaccagagagagcggtgtacctgatagatttaccggaagcgggagcggaacagattttacacttactatttcctctgtccaggccgaagacctcgcggtgtactactgtcagaatgactattcttacccgctgaccttcggggccggaacgaagctggagttgaaaggatcaggaggtgggggcagctgcccgtacagtaatccaagtctctgctccggaggcggcggctcatgcccatattctaatccatcactttgcagcggcgggggtggtagcactaccacaccagcacctcgaccaccaactccagcaccaaccattgcatcacagccactgagcctgcgaccagaggcctgtagacctgctgcaggaggagctgtgcatacccgaggactggactttgcctgcgatatctacatttgggctcctctggcaggaacttgtggcgtgctgctgctgtccctggtcatcaccctgtactgcaagcgaggccggaagaaactgctgtatattttcaaacagccctttatgagacctgtgcagactacccaggaggaagacggctgcagctgtaggttccccgaggaagaggaaggcgggtgtgagctgagggtcaagtttagccgctccgcagatgcccctgcttaccagcaggggcagaatcagctgtataacgagctgaatctgggacggagagaggaatacgacgtgctggataaaaggcgcgggagagaccccgaaatgggaggcaagccacgacggaaaaacccccaggagggcctgtacaatgaactgcagaaggacaaaatggcagaggcctatagtgaaatcgggatgaagggagagagaaggcgcggcaaagggcacgatggcctgtaccaggggctgtctactgccaccaaggacacctatgatgctctgcatatgcaggcactgcctccaagg

SEQ ID NO: 209 - protein, 291 aa, homo sapiens
  MESOTHELIN target antigen region
EVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQAPRRPLPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGL

SEQ ID NO: 210 - protein, 116 aa, artificial
  MESO1 heavy chain variable region
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCARVDYKAFDIWGQGTMVTVSS

SEQ ID NO: 211 - protein, 107 aa, artificial
  MESO1 light chain variable region
DIQMTQSPSSLSASVGDRVTITCRASQGIRNDLHWYQQKPGKAPKRLIYAASSLQSGVPSRFSGSGSGTEFTLTISSLQPEDFATYYCLQHYSYPWTFGQGTKVEIK

SEQ ID NO: 212 - protein, 121 aa, artificial
  MESO2 heavy chain variable region
QVQLVQSGAEVKKPGESLKISCKGSGYSFTNYWIGWVRQMPGKGLEWMGVIMPSDSYTRYSPSFQGQVTISADKSISTAYLQWSSLKASDTAMYYCARYGHGMYGGALDVWGQGTLVTVSS

SEQ ID NO: 213 - protein, 111 aa, artificial
  MESO2 light chain variable region
DIVLTQSPATLSLSPGERATLSCRASQSVRSSRLAWYQQKPGQAPRLLIYGASKRATGVPARFSGSGSGTDFTLTISSLEPEDFAVYYCQQYSHDPSGTFGQGTKVEIKRT

SEQ ID NO: 214 - protein, 127 aa, artificial
  P4 heavy chain variable region
QVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILG

SEQ ID NO: 215 - protein, 116 aa, artificial
  P4 light chain variable region
QPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLS

SEQ ID NO: 216 - protein, 5 aa, artificial
  CDRH1-Meso1
SYYWS

SEQ ID NO: 217 - protein, 16 aa, artificial
  CDRH2- meso1
YIYYSGSTNYNPSLKS

SEQ ID NO: 218 - protein, 8 aa, artificial
  CDRH3-meso1
VDYKAFDI

SEQ ID NO: 219 - protein, 11 aa, artificial
  CDRL1- meso1
RASQGIRNDLH

SEQ ID NO: 220 - protein, 7 aa, artificial
  CDRL2- meso1
AASSLQS

SEQ ID NO: 221 - protein, 9 aa, artificial
  CDRL3- meso1
LQHYSYPWT

SEQ ID NO: 222 - protein, 482 aa, artificial
  MESO1 CAR full sequence
MALPVTALLLPLALLLHAARPQVQLQESGPGLVKPSETLSLTCTVSGGSISSYYWSWIRQPPGKGLEWIGYIYYSGSTNYNPSLKSRVTISVDTSKNQFSLKLSSVTAADTAVYYCARVDYKAFDIWGQGTMVTVSSGGGGSGGGGSGGGGSDIQMTQSPSSLSASVGDRVTITCRASQGIRNDLHWYQQKPGKAPKRLIYAASSLQSGVPSRFSGSGSGTEFTLTISSLQPEDFATYYCLQHYSYPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 223 - protein, 925 aa, artificial
  TGFBR2 TALEN Left
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNGGGKQALETVQRLLPVLCQDHGLTPDQVVAIASNIGGKQALETVQALLPVLCQDHGLTPDQVVAIASNGGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 224 - protein, 925 aa, artificial
  TGFBR2 TALEN Right
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNNGGKQALETVQRLLPVLCQDHGLTPDQVVAIASNIGGKQALETVQALLPVLCQDHGLTPDQVVAIASNNGGKQALETVQRLLPVLCQDHGLTPDQVVAIASNIGGKQALETVQALLPVLCQDHGLTPDQVVAIASNIGGKQALETVQALLPVLCQDHGLTPDQVVAIASNNGGKQALETVQRLLPVLCQDHGLTPDQVVAIASNIGGKQALETVQALLPVLCQDHGLTPDQVVAIASNGGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASHDGGKQALETVQRLLPVLCQDHGLTPQQVVAIASNGGGRPALESIVAQLSRPDPALAALTNDHLVALACLGGRPALDAVRRGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 225 - DNA, 59 bp, homo sapiens; identical to SEQ ID NO: 81
  TRAC target sequence

SEQ ID NO: 226 - DNA, 49 bp, homo sapiens; identical to SEQ ID NO: 82
  PD1 target sequence

SEQ ID NO: 227 - DNA, 47 bp, homo sapiens; identical to SEQ ID NO: 83
  B2M target sequence

SEQ ID NO: 228 - protein, 118 aa, artificial
  MUC1-A VH humanized H1
QVQLVQSGAEVKKPGASVKVSCKASGYTFTNYGLSWVRQAPGQGLEWIGENHPGSGIIYHNQKFQGRVTMTRDTSTSTVYMELSSLRSEDTAVYYCARSSGTRGFAYWGQGTLVTVSS

SEQ ID NO: 229 - protein, 118 aa, artificial
  MUC1-A VH humanized H2
QVQLVQSGAEVKKPGASVKVSCKASGYTFTNYGLSWVRQAPGQGLEWIGENHPGSGIIYHNQKFQGRVTMTADTSTSTVYMELSSLRSEDTAVYYCARSSGTRGFAYWGQGTLVTVSS

SEQ ID NO: 230 - protein, 118 aa, artificial
  MUC1-A VH humanized H3
QVQLVQSGAEVKKPGASVKVSCKASGYTFTNYGLSWVRQAPGQGLEWIGENHPGSGIIYHNQKFQGRVTMTRDKSTSTVYMELSSLRSEDTAVYYCARSSGTRGFAYWGQGTLVTVSS

SEQ ID NO: 231 - protein, 118 aa, artificial
  MUC1-A VH humanized H4
QVQLVQSGAEVKKPGASVKVSCKASGYTFTNYGLSWVRQAPGQGLEWIGENHPGSGIIYHNQKFQGRVTMTADKSTSTVYMELSSLRSEDTAVYYCARSSGTRGFAYWGQGTLVTVSS

SEQ ID NO: 232 - protein, 118 aa, artificial
  MUC1-A VH humanized H5
QVQLVQSGAEVKKPGASVKVSCKASGYTFTNYGLSWVRQAPGQGLEWIGENHPGSGIIYHNEKFRGRVTMTADTSTSTVYMELSSLRSEDTAVYYCARSSGTRGFAYWGQGTLVTVSS

SEQ ID NO: 233 - protein, 118 aa, artificial
  MUC1-A VH humanized H6
QVQLVQSGAEVKKPGASVKVSCKASGYTFTNYGLSWVRQAPGQGLEWIGENHPGSGIIYHNEKFRGRVTMTRDKSTSTVYMELSSLRSEDTAVYYCARSSGTRGFAYWGQGTLVTVSS

SEQ ID NO: 234 - protein, 118 aa, artificial
  MUC1-A VH humanized H7
QVQLVQSGAEVKKPGASVKVSCKASGYTFTNYGLSWVRQAPGQGLEWIGENHPGSGIIYHNEKFRGRVTMTADKSTSTVYMELSSLRSEDTAVYYCARSSGTRGFAYWGQGTLVTVSS

SEQ ID NO: 235 - protein, 114 aa, artificial
  MUC1-A VL humanized L1
DVVMTQSPLSLPVTLGQPASISCRSSQSIVHSNGNTYLEWFQQRPGQSPRRLIYKVSNRFSGVPDRFSGSGSGTDFTLKISRVEAEDVGVYYCFQGSHGPWTFGQGTKVEIKRA

SEQ ID NO: 236 - protein, 114 aa, artificial
  MUC1-A VL humanized L2
DVVMTQSPLSLPVTLGQPASISCRSSQSIVHSNGNTYLEWYQQRPGQSPRRLIYKVSNRFSGVPDRFSGSGSGTDFTLKISRVEAEDVGVYYCFQGSHGPWTFGQGTKVEIKRA

SEQ ID NO: 237 - protein, 114 aa, artificial
  MUC1-A VL humanized L3
DVVMTQSPLSLPVTLGQPASISCRSSQSIVHSNGNTYLEWFQQRPGQSPRLLIYKVSNRFSGVPDRFSGSGSGTDFTLKISRVEAEDVGVYYCFQGSHGPWTFGQGTKVEIKRA

SEQ ID NO: 238 - protein, 114 aa, artificial
  MUC1-A VL humanized L4
DVVMTQSPLSLPVTLGQPASISCRSSQSIVHSNGNTYLEWYQQRPGQSPRLLIYKVSNRFSGVPDRFSGSGSGTDFTLKISRVEAEDVGVYYCFQGSHGPWTFGQGTKVEIKRA

SEQ ID NO: 239 - protein, 114 aa, artificial
  MUC1-A VL humanized L5
DVLMTQSPLSLPVTLGQPASISCRSSQSIVHSNGNTYLEWYQQRPGQSPRLLIYKVSNRFSGVPDRFSGSGSGTDFTLKISRVEAEDVGVYYCFQGSHGPWTFGQGTKVEIKRA

(19) United States
(12) Patent Application Publication
(10) Pub. No.: US 2024/0197880 A1
(43) Pub. Date: Jun. 20, 2024

(54) NEW ANTI-MUC1 CARS AND GENE EDITED IMMUNE CELLS FOR SOLID TUMORS CANCER IMMUNOTHERAPY

(71) Applicant: CELLECTIS S.A., Paris (FR)

(72) Inventors: Beatriz ARANDA-ORGILLES, Paris (FR); Tomasz KURCON, Paris (FR); Laurent POIROT, Paris (FR)

(21) Appl. No.: 18/556,957

(22) PCT Filed: Apr. 29, 2022
(86) PCT No.: PCT/EP2022/061532

§ 371 Date: Oct. 24, 2023

Related U.S. Application Data
(60) Provisional application No. 63/182,330, filed on Apr. 30, 2021.

(30) Foreign Application Priority Data
Jul. 6, 2021 (DK) PA 202170361

(51) Int. Cl. A61K 39/00; A61P 35/00; C07K 14/725; C07K 16/30; C12N 5/0783; C12N 15/90

(52) U.S. Cl. CPC A61K 39/46447; A61K 39/4611; A61K 39/4613; A61K 39/4631; A61P 35/00; C07K 14/7051; C07K 16/3092; C12N 5/0636; C12N 15/907; C07K 2317/622; C07K 2319/03; C07K 2319/30; C12N 2501/2302

(57) ABSTRACT

The present invention relates to genetically engineered immune cells expressing new anti-MUC1 chimeric antigen receptors and their use in the treatment of solid tumors, particularly suited for allogeneic cell immunotherapy.

### REFERENCE TO SEQUENCE LISTING SUBMITTED ELECTRONICALLY

The instant application contains a Sequence Listing, which has been submitted electronically in ASCII format and is hereby incorporated by reference in its entirety. Said ASCII copy, created on Dec. 27, 2023, is named 18556957_SL.txt and is 225,442 bytes in size.

### FIELD OF THE INVENTION

The present invention relates to the field of cell immunotherapy and more particularly to engineered immune cells expressing anti-MUC1 chimeric antigen receptors (CAR) useful in the treatment of solid tumors.

### BACKGROUND OF THE INVENTION

CAR Immune cell therapy, mainly T-cells and NK-cells, has revolutionized treatment of hematological cancers over the last ten years. However, so far, the same type of therapy has failed to be an effective way of treating solid tumors [June et al., CAR T cell immunotherapy for human cancer (2018) Science 359:1361-1365].

Indeed, challenges in treating solid tumors arise from two major areas: 1) off-tumor toxicity due to the lack of exclusive tumor specificity and 2) complex immunosuppressive biology of solid tumors [Marofi, F., et al. (2021) CART cells in solid tumors: challenges and opportunities. Stem Cell Res Ther. 18(4):843-851].

Specificity of CAR-immune cells remains a critical challenge due to on-target off-tumor activities resulting in damage to healthy tissues. In this respect, the efficiency of therapeutic antibodies in immunotherapy has proven to be of low predictive value on the specificity/activity of CAR-T cells using the same ScFv as that as previously developed antibodies. The opposite is also true, since anti-CD19 CARs have proven to be highly efficient where CD19 antibodies had failed to be developed against hematological cancers.

In hematological tumors off-target damages carry lower risks. In general, the associated toxicities are well tolerated or effectively managed. This is partly due to antigens being restricted to specific cell lineages of the hematological compartment such as CD19 on B-cells. Loss of healthy B-cells due to CD19 CAR-T activity can results in B-cell aplasia, which carries a higher risk of infections, but is otherwise non-lethal and can be well managed with intravenous immunoglobulin (IVIG) replacement therapy.

In stark contrast, expression of CAR-T target antigen on solid tissues are associated with lethal toxicities. For example, anti-HER2 CAR-T cells administered for the treatment of metastatic colon cancer, attacked low HER2 expressing lung epithelium resulting in a fatal event [Morgan, R. A. et al. (2010) Case report of a serious adverse event following the administration of T cells transduced with a chimeric antigen receptor recognizing ERBB2. Mol. Ther. 18(4):843-851]. Similar toxicity was observed in therapy against renal cancer in which CAR-T cells against CAIX caused liver damage by attacking CIAX expressing bile duct epithelia [Lamers C H, et al. (2006) Treatment of metastatic renal cell carcinoma with autologous T-lymphocytes genetically retargeted against carbonic anhydrase IX: first clinical experience. Journal of Clinical Oncology. 24(13):e20-2]. Overall, the success of CAR-T therapies against solid tumors is limited, and in instances, where measurable responses were observed, dose escalation as a means to improve efficacy was not possible, partially due to toxicities associated with off-target binding [Wagner, J. (2020) CAR T Cell Therapy for Solid Tumors: Bright Future or Dark Reality? Molecular Therapy. 28(11):2320-23394].

An additional level of failure to obtain significant responses in solid-tumors, even for the most active and specific scFVs, comes from the tumor microenvironment in which: (1) CAR-immune cell infiltration is restricted due to extracellular matrix deposition and high interstitial pressure, and (2) Intratumoral CAR-immune cells expansion and effector functions are blocked due to hypoxia and lack of nutrients. 3) immunosuppressive signals produced by other tumor resident cells including regulatory T-cells (T-reg: TGF-R), cancer associated fibroblasts (CAF: TGF-β) and Myeloid derived suppressor cells (MDSC: IL-10 and TGF-β) prevent CAR-T cell expansion and effector functions. 4) upregulation of immune checkpoint ligands such as PDL1 directly inhibit CAR-T cells activity. In order to develop successful CAR-T product for the treatment of solid tumors these challenges need to be addressed through complex engineering of next generation CAR-T cells.

TABLE 1
Determinants of successful CAR-T Therapy in solid tumors
Determinants of successful CAR-T Therapy in solid tumors

| Determinant | Main effect | Challenges |
|---|---|---|
| Coverage | Tumor clearance | Vast majority of cells within tumor |
| | efficiency | should express target antigen |
| Specificity | Limited “Off | It should be limited to tumor tissue, |
| | tumor” | unless the damage caused by off-target |
| | cytotoxicity | toxicity can be tolerated or well |
| | | managed |
| Stability | Response | The expression should be stable and |
| | duration and | not easily regulated by external signals |
| | recurrence | It plays an important role in the growth |
| | | and survival of cancer cells |

These challenges, which are recapitulated in Table 1, are tackled in the present invention by engineering anti-MUC1 CAR-immune cells that target highly specific post-translational versions of MUC1 epitopes exclusive to tumors and by introducing genetic modifications that enable them to combat the immunosuppressive tumor microenvironment associated with MUC1 positive solid tumors.

MUC1 is a large mucin type glycoprotein produced by epithelial cells lining several organs including lung, cervix, stomach and the intestines. In normal tissue MUC1 is localized only to the apical side of the epithelial lining, extending into the lumen where the glycans on MUC1 trap water forming a mucosal barrier that protects these tissues from damage and pathogens. MUC1 similarly to other solid tumor targets is highly enriched in tumors, however unlike other solid tumor targets, MUC1 is structurally different when produced by tumor cells (herein abbreviated as tMUC1). The structural differences in MUC1 arise due to differential glycosylation of MUC1 within the VNTR region of the extracellular subunit (see FIG. 1). O-glycans appended to the tMUC1 are truncated, missing entirely or they are prematurely modified with sialic acid. Consequently, tMUC1 produced by tumor cells is different on a molecular level and scFVs produced against underglycosylated tMUC1 should not recognize fully glycosylated MUC1 produced by normal cells. Based on these differences between tMUC1 and MUC1, It has been hypothesized that CAR-immune cells produced against tMUC1 would be highly specific at recognizing tumors [Naito et al. (2017) Generation of Novel Anti-MUC1 Monoclonal Antibodies with Designed Carbohydrate Specificities Using MUC1 Glycopeptide Library. ACS Omega. 2(11): 7493-7505].

Based on a primary selection of 14 anti-MUC1 ScFvs candidates from the literature, the inventors have developed CAR scaffolds against tMUC1 by including 4 ScFvs that commonly target the same polypeptide segment of MUC1. The expression of the different CARs in immune cells have all resulted in positive and specific anti-tMUC1 anti-tumor response activities. However, the CARs showed in-vivo anti-tumor responses to different extend.

Since CAR-immune cell activity in solid tumors is often inhibited due to the tumor immunosuppressive environment, the inventors have expressed the different CARs of the invention in immune cells that have been engineered to simultaneously tackle several issues associated with solid tumors, such as:
1) Increasing potency of CAR-immune cells by cytokine induced heterologous expression;
2) Limiting CAR-T cells exhaustion by inactivating immune checkpoints genes that inhibit CAR-immune cells activation;
3) Preventing immune suppression from tumor microenvironment by using alternative for mitigating immunosuppressive effects of TGF-β;
4) Enhancing tumor infiltration by expressing Hyaluronan (HA), which is a glycosaminoglycan that is an integral structural and signaling component of the extracellular matrix; and/or
5) Mitigating hypoxic and low nutrient environment by overexpressing metabolic enzymes, for example Glucose-6-Phosphate Isomerase (ex: GP11), Lactate dehydrogenase (ex: LDHA) and phosphoenolpyruvate carboxykinase 1 (ex: PCK1) to boost, among others, the proliferative ability of inflammatory Th17 cells in hypoxic and nutrient deficient environments.
   During those experiments, gene targeting integration, as well as retroviral approaches, have been used and combined to finally optimize the genetic engineering of anti-MUC1 CAR immune cells and provide therapeutic cells with improved potency.

### SUMMARY OF THE INVENTION

To achieve the present invention, four different CARs have been synthetized against tMUC1: CLS MUC1-A, CLS MUC1-B, CLS MUC1-C and CLS MUC1-D. The scFVs used in these CAR constructs recognizing different epitopes of tMUC1 polypeptide segment of SEQ ID NO:1, which were screened to be breast cancer cells specific (HCC70 and T47D cell lines). As a negative control MUC1/tMUC1 negative HEK293FT cells were used. As shown in the experimental part herein the four different CARs exhibited high cytotoxicity activity in vitro against breast cancer cell lines T47D and HCC70, while not being cytotoxic against normal primary cells from lung, kidney or cervix. These data demonstrated that the four CARs produced were highly specific for tMUC1. Meanwhile, the inventors could observe that the four CARs, when expressed into primary T-cells conferred different level of activities and that one of them (CAR MUC1-A) unexpectedly overpassed the others in terms of anti-tumor activity in-vivo.

The above anti-MUC1 CARs of the invention, which were all primarily considered as appropriate for eliciting a specific immune response against solid tumors, have been tested in immune cells which have been genetically engineered, in particular in view of their applicability to allogeneic settings.

The results show that anti-MUC1CARs—and not only the four CARs detailed herein—can be successfully combined with a range of genetic attributes to produce sophisticated gene edited CAR immune cells with significantly improved anti-tumor potency.

The present invention thus encompasses new anti-MUC1 CARs, as well as the production of engineered immune cells endowed with anti-MUC1 CARs displaying a range of genetic attributes that potentiate the activity of said CARs for the in-vivo elimination of solid tumors.

An anti-MUC1 chimeric antigen receptor (CAR) according to the present invention is preferably directed against an antigen that can discriminate low glycosylated epitopes of the MUC1 polypeptide region HGVTSAPDTRPAPGSTAPPA (SEQ ID NO:20).

One exemplary and preferred anti-MUC1 CAR of the invention is one including:
a transmembrane domain;
a cytoplasmic domain comprising a CD3 zeta signalling domain and a co-stimulatory domain
a ligand binding-domain comprising a ScFv having at least 80%, preferably at least 90%, more preferably at least 95%, and even more preferably at least 99% sequence identity with one selected from MUC1-A (SEQ ID NO:17), MUC1-B (SEQ ID NO:27), MUC1-C(SEQ ID NO:37) or MUC1-D (SEQ ID NO:37).

The structure of such CARs used in the present invention generally pertains to second third or fourth generation as described in the art [Subklewe, M., et al. (2019) Chimeric Antigen Receptor T Cells: A Race to Revolutionize Cancer Therapy. Transfusion medicine and hemotherapy. 46(1), 15-24] by combining the anti-tMUCI ligand binding domain with a hinge and transmembrane domain from CD8alpha, together with a co-stimulatory domain from 4-11BE and a signalling domain from CD3 zeta.

The invention also pertains to the polypeptide and polynucleotide sequences as well as the vectors used to introduce and express the anti-MUC1 CARs at the surface of the immune cells.

The engineered immune cells expressing anti MUC1-CARs as per the present invention are generally NK or T-cells or precursors thereof, originating from human donors, suitable for use in allogeneic settings. Such cells can be gene-edited in such a way they are less immunoreactive towards the patient's host cells and in the same time more aggressive towards the patient's tumors cells. In this regard, they can advantageously combine several or all the gene modifications that are illustrated in FIGS. 5, 6, 7 and/or 15.

According to some embodiments expression of TCRalpha and/or TCRbeta can be repressed or inactivated in said engineered cells.

According to some embodiments said engineered immune cells can be mutated to confer resistance to at least one immune suppressive drug, such as an anti-CD52 antibody.

According to some embodiments, said engineered immune cells can be mutated to confer resistance to at least one chemotherapy drug, in particular a purine analogue drug.

According to some embodiments, said engineered immune cells can be mutated to improve its persistence or its lifespan into the patient, in particular into a gene encoding MHC-I component(s) such as HLA or B2m.

According to some embodiments, said engineered immune cells can be mutated to improve its MUC1 CAR-dependent immune activation, in particular to reduce or suppress the expression of immune checkpoint proteins and/or receptors thereof, such as PD-1/PDL1, CTLA4 and/or TIM3.

According to some embodiments, said engineered immune cells can be mutated into a gene involved into TGFbeta pathway, such as one encoding TGFbeta and/or TGFbeta receptor (TGFβRII).

According to some embodiments, said engineered immune cells can express a decoy of TGFbeta receptor, such as a dominant negative TGFbeta receptor (dnTGFβRII) having at least 80% polypeptide sequence identity with SEQ ID NO.59.

According to some embodiments, said engineered immune cells are transfected with an exogenous polynucleotide allowing the co-expression of the CAR and of the decoy of TGFbeta receptor, comprising for instance: a first polynucleotide sequence encoding said anti-MUC1-specific CAR, a second polynucleotide encoding a 2A self-cleaving peptide, and a third polynucleotide encoding said dominant negative TGFbeta receptor (dnTGFβRII).

According to some embodiments, said engineered immune cells are transfected with exogenous polynucleotide sequences allowing the co-expression of an anti-MUC1 chimeric antigen receptor (CAR) with another polypeptide having showing identity with one selected from:
NK cell inhibitor, such as HLAG, HLAE or ULBP1;
CRS inhibitor, such as is a mutated IL6Ra, sGP130 or IL18-BP; or
Cytochrome(s) P450, CYP2D6-1, CYP2D6-2, CYP2C9, CYP3A4, CYP2C19 or CYP1A2, conferring hypersensitivity of said immune cells to a drug, such as cyclophosphamide and/or isophosphamide,
Dihydrofolate reductase (DHFR), inosine monophosphate dehydrogenase 2 (IMPDH2), calcineurin or methylguanine transferase (MGMT), mTORmut or Lckmut, conferring drug resistance
Chemokine or a cytokine, such as IL-12, IL-15 or IL-18;
Hyaluronidase, such as HYAL1, HYAL2 and HYAL3 (SPAM1);
Chemokine receptors, such as CCR2, CXCR2, or CXCR4;
a secreted inhibitor of Tumor Associated Macrophages (TAM), such as a CCR2/CCL2 neutralization agent, to enhance the therapeutic activity of the immune cells; and/or
metabolic enzymes such as: glucose phosphate isomerase 1 (GP11), Lactate deshydrogenase (LDHA) and/or phosphoenolpyruvate carboxykinase 1 (PCK1).

The invention also provides with methods for manufacturing populations of the above engineered therapeutic immune cells useful for the treatment of solid tumors. Such methods can typically comprise the steps of:
a) Providing immune cells originating from a patient or preferably from a donor;
b) Expressing in said cells an anti-MUC1 chimeric antigen receptor (CAR);
c) Introducing at least one genetic modification(s) in the genome of said cell, said modification(s) being selected from those leading to:
   Reduced or inactivated TCR expression;
   Reduced or inactivated B2M;
   Reduced interaction between TGFβ and TGFβRII;
   Reduced interaction between PD1 and PDL1; and/or
   Enhanced IL-12, IL-15 or IL-18 expression;
d) Expanding said cells to form a population of therapeutically effective population of immune cells.

According to some embodiments, the genetic modifications, especially to knock-out gene expression, are performed by using sequence specific gene editing reagents, such as rare-cutting endonucleases/nickases or base editors.

The combinations of the above gene editing modifications have been found particularly relevant to overcome the biochemical barriers raised by tumors preventing access of immune cells, in particular T-cells, to hot tumor cells.

The resulting populations of cells generally comprise at least 25%, preferably at least 50%, more preferably at least 75% of engineered cells having at least two, preferably at least three, preferably at least four, even more preferably at least five of said genetic modification(s).

In some embodiments, the therapeutic compositions can comprise a population of engineered immune cells characterized by one, several or all the following (phenotypic) attributes:
Exogenous expression of a CAR targeting a tMUC1 epitope, and
Reduced B2M expression by at least 30%; preferably 50%, more preferably 75%; and/or
Reduced PD1 expression by at least 30%; preferably 50%, more preferably 75%;
and/or
Optionally, reduced TCR expression by at least 50%; preferably 75%;
Optionally, increased IL-12, IL-15 or IL-18 expression by at least 50%; preferably 75, more preferably 100%;
Optionally reduced TGFβ or TGFβRII expression, by at least 30%; preferably 50%, more preferably 75%;
Optionally, exogenous expression of a decoy of TGFβR2,
Optionally secretion of HYAL1, HYAL2 and HYAL3 (SPAM1) by introduction of exogenous coding sequences; and
Optionally expression of GP11, PCK1 and/or LDHA by introduction of exogenous coding sequences.

From the genotypic perspective, such therapeutic populations of engineered immune cells are characterized by one, several or all of the following (genotypic) attributes:
At least 50% of the immune cells displaying an exogenous polynucleotide sequence encoding a CAR targeting a tMUC1 epitope; and
At least 50% of the immune cells display B2M inactive allele(s) preferably at least 75%;
and/or
At least 30% of the immune cells display mutated PD1 allele(s), preferably at least 50%, more preferably 75%;
Optionally, at least 50% of T-cells display TCR inactive allele(s), preferably at least 75%;
Optionally, at least 30% of the immune cells display exogenously introduced sequences encoding IL-12, IL-15 or IL-18, preferably at least 50%, more preferably 75%;
Optionally, at least 20% of the immune cells display sequences encoding a decoy of TGFβR2 exogenously inserted in their genome, preferably at least 50%, more preferably 75%;
Optionally, at least 20% of the immune cells display sequences encoding HYAL1, HYAL2 and/or SPAM1 exogenously inserted in their genome, preferably at least 50%, more preferably 75%;
Optionally at least 20% of the immune cells display sequences encoding GP11 and/or PCK1 exogenously inserted in their genome, preferably at least 50%, more preferably 75%;
Optionally, at least 30% of the immune cells display mutated allele(s) encoding TGFβ or TGFβRII, preferably at least 50%, more preferably 75%; The engineered immune cells according to the present invention are particularly suited for treating a condition characterized by tMUC1 expressing cells, in particular solid tumors, such as typically: oesophageal cancer, breast cancer, especially triple negative breast cancer, gastric cancer, cholangiocarcinoma, pancreatic cancer, colon cancer, lung cancer, thymic carcinoma, mesothelioma, ovarian cancer and/or endometrial cancer.

The invention thus encompasses methods for producing engineered cells, the resulting therapeutic cells, populations of cells comprising such cells and therapeutic compositions comprising same, as well as the methods of treatment allowing to address pathologies induced by tMUC1 expressing cells.

The whole method of treatment typically comprises the steps of:
Engineering immune cells from the patient or from a donor to express a functional anti-MUC1 CAR
Administrating said CAR positive engineered immune cells to the patient to eliminate cells expressing a tMUC1 epitope.

In allogeneic settings, but not only, such methods of treatment can advantageously combine the administration of (1) a lymphodepleting agent and (2) a population of allogeneic engineered immune cells expressing a chimeric antigen receptor (CAR) specifically directed against a tMUC1 epitope.

In such settings, it can be advantageous that anti-MUC1 CAR positive engineered immune cells are inactivated in its CD52 gene expression, or any other polypeptide targeted by a lymphodepleting agent, to confer resistance to lymphodepletion treatment.

The invention also provides to utilize dual CARs carrying either a combination of two anti-tMUC1 scFVs which might target a larger number of cancers and eliminate antigen escape due to changes in glycoforms under selective pressure of CAR-T therapy. It also encompasses the use of dual CAR T-cells targeting two independent antigens of MUC-1 to increase the rate of target cell recognition and efficiency of the CAR-T cell treatment. In a preferred approach, anti-MUC1 CAR is co-expressed with an anti-MESOTHELIN CAR in engineered T-cells.

### DESCRIPTION OF THE FIGURES

FIG. 1: A. Schematic representation of MUC1 expression on the surface of normal and tumor cells. B. Schematic comparative representation of the glycosylation status of MUC1 on healthy and tumor cells. Cancer-Associated MUC1 is under-glycosylated along tandem polypeptide repeats (SEQ ID NO:20).

FIG. 2: Specificity of the selected MUC1 scFVs measured on cancer cells and on primary cells from kidney, lung and cervical tissues. By contrast to the breast cancer cell lines (T47D and HCC70), these later, which express high level of normal MUC1, are not stained by the candidate scFVs.

FIG. 3: Diagrams showing the percentage of specific lysis of the respective breast cancer cells T47D and HCC70 obtained by expression of the anti-MUC1-CAR of the present invention in primary T-cells in-vitro (at ratios 1:1, 2.5:1 and 5:1). The diagrams show high activity response of the new anti-MUC1 CAR-T, and superiority of CLS MUC1-A CAR.

FIG. 4: Analysis of solid tumor microenvironment and of different factors that inhibit CAR-T function in solid tumors.

FIG. 5: Schematic representation of an anti MUC1-CAR engineered immune cell according to the present invention with its main characteristic genetic attributes. A. Representation of a genetic construct for co-expression of the anti-MUC1 CAR along with a decoy for TGFbeta (dnTGRBR2B). This genetic construct can be included into a lentiviral vector or on a donor template for site-directed gene insertion by homologous recombination or NHEJ using a nuclease or nickase gene editing reagent. B. Representation of genetic insertion at the B2m endogenous locus, leading to inactivation of β2m and expression of HLA-E that provides resistance against patient's NK cells. C. Representation of genetic insertion at the PDCD1 (PD1) endogenous locus, leading to inactivation of PD1 and expression of IL-12 that boost immune cells activity. D. Representation of the engineered immune cell surface showing the heterologous expression of the anti-MUC1 CAR, HLA-E as a NK inhibitor, secretion of IL-12 and dnTGFβ RII as a decoy for TGFβ. On another hand, expression of TCR, PD1 and β2m is repressed or inactivated.

FIG. 6: Detailed mechanism of the AB2M-HLA-E genetic attribute displayed in the above FIG. 5B.

FIG. 7: Detailed mechanism of the APD1-IL12 genetic attribute displayed in the above FIG. 5C. By inserting the exogenous sequence encoding IL-12 under transcriptional control of the endogenous PD1 promoter, secretion of IL-12 gets synchronized with tumor antigen recognition by the CAR.

FIG. 8: FACS analysis of the cells engineered as per the present invention showing that at least 30% of the cells in the population of engineered cells show at least three of the main genetic attributes (details in the experimental section). At least 30% of the cells in the population were tested CAR positive, [PD1]negative, [TCR]negative, [β2m]negative and [HLAE] positive.

FIG. 9: Low score of candidate off-sites observed in the engineered cells of the invention (rLV CAR-DNTGFBR2 transduction+Triple TALEN© transfection (TRAC/B2M/PD-1)+AAV-mediated KI of HLA-E) when performing OCA (Oligo Capture assay) analysis.

FIG. 10: Diagrams showing experimental results as detailed in Example 2 showing strong in vivo intra-tumoral expansion of UCARTMUC1 achieved with APD1-IL12 genetic attribute against HCC70 cancer cells (same experimental set up as illustrated in FIG. 12) FIG. 11: Graph showing in-vivo tumor volume from their inoculation to day 28 in mice treated respectively with the four anti-MUC1 CAR-T of the present invention.

FIG. 12: Experiments showing that the genetic attributes detailed in FIG. 5 extend mice survival when treated with anti-MUC1 CAR (see example 2).

FIG. 13: Graph showing subcutaneous tumor volume reduction obtained by treatment with CLS MUC1-A CAR-T cells in mice experiments.

FIG. 14: Analysis of HCC70 tumors from the mice treated with anti-MUC1 CAR T-cells with and without genetic attributes. The graphs show stronger intratumoral UCARTMUC1 expansion achieved with cells bearing the genetic attributes according to the invention.

FIG. 15: Principle of anti-MUC1 CAR/anti-MESOTHELIN CAR dual approach aiming to overcome solid tumors heterogeneity.

FIG. 16: Principle of the anti-MUC1 CAR-T attributes according to the invention for an optimized immune scenario.

FIG. 17: Schematic representation of an optimized anti MUC1-CAR engineered immune cell according to the present invention with its main characteristic genetic attributes. The anti-MUC1 CAR construct can be introduced randomly (with lentiviral vector) or at a specific site (by homologous recombination or NHEJ using a gene editing reagent); HLA-E is integrated at the β2m endogenous locus leading to host immune escape; IL-12 is integrated at the PDCD1 (PD1) endogenous locus, leading to PD1 inactivation and tumor-specific and localized IL-12 expression, TGFBR2 is inactivated leading to TGF p pathway blockade.

FIG. 18: A. Experimental in vivo set up for MUC1-A and MUC1-C engineered CAR-T cells evaluation. B. Tumor volume obtained after treatment using the indicated doses of MUC1-A and MUC1-C engineered CAR-T cells. C. Survival Curve. D. Percentage of hCD45+ cells detection in tumors 54 days post treatment. These results reveal superiority and dose response sensitivity of MUC1-A over the other anti-tMUC1 CARs.

FIG. 19: IHC results performed on tumor microarrays using MUC1-A, MUC1-C and MUC1-D scFVs proteins, revealing that CAR MUC1-A shows more affinity to samples of patients tumor cells.

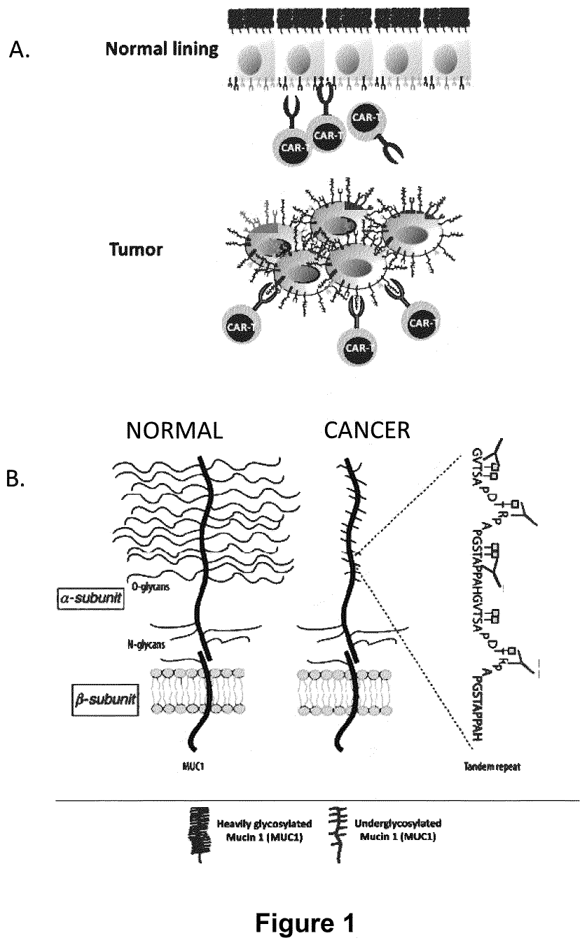

### DETAILED DESCRIPTION OF THE INVENTION

Unless specifically defined herein, all technical and scientific terms used herein have the same meaning as commonly understood by a skilled artisan in the fields of gene therapy, biochemistry, genetics, and molecular biology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail. Further, the materials, methods, and examples are illustrative only and are not intended to be limiting, unless otherwise specified.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, New York: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986).

The present invention is drawn to a general method of treating solid tumors by adoptive immune cells directed against underglycosylated MUC1 tumor antigens, in particular the specific epitope region spanning the polypeptide sequence SEQ ID NO:1 of this polypeptide, and more particularly by using engineered allogeneic CAR immune cells, which have proven particular efficiency in targeting such antigens.

Herein is described a method for producing engineered immune cells directed against human MUC1 protein (also referred to as MUCIN-1 and P15941 in the Uniprot database), and more particularly the polypeptide region represented by SEQ ID NO:1, which is underglycosylated at the surface of malignant cells. As shown in the experimental section in the present specification, efficient CAR T-cells have been produced by directing CARs against this antigen region comprising or consisting of SEQ ID NO:1, in particular by using one of the binding domains including the scFvs comprising SEQ ID NO:17 (MUC1-A), SEQ ID NO:27 (MUC1-B), SEQ ID NO:37 (MUC1-C) or SEQ ID NO:47 (MUC1-D).

The resulting engineered immune cells according to the invention, in general NK or T-cells armed with the anti-MUC1 CAR of the present invention have shown higher activation, potency, killing activity, cytokine release, and in-vivo persistence than their counterparts endowed with other prior anti-MUC1 CARs.

The present invention thus is drawn to immune cells endowed with a CAR targeting specific epitope(s) comprised in the sequence SEQ ID NO:1 of MUC1, especially immune cells engineered for treating solid tumors, such as triple negative breast cancer tumors.

### Design of Anti-MUC1-CARs for Expression in Immune Cells:

By “Chimeric Antigen Receptor (CAR)” is meant recombinant receptors comprising a targeting moiety that is associated with one or more signaling domains in a single fusion molecule. In general, the binding moiety of a CAR consists of an antigen-binding domain of a single-chain antibody (scFv), comprising the light and heavy variable fragments of a monoclonal antibody joined by a flexible linker. These ScFv are generally characterized by complementarity determining regions CDRs, which are short invariable polypeptide sequences crucial for the specificity of interaction with the epitope region. Binding moieties based on receptor or ligand domains have also been used successfully. The signaling domains of CARs are generally derived from the cytoplasmic region of the CD3zeta or the Fc receptor gamma chains, which are generally combined with signaling domains from co-stimulatory molecules including CD28, OX-40 (CD134), ICOS and 4-1BB (CD137) to enhance survival and increase proliferation of the cells. CARs are generally expressed in effector immune cells to redirect their immune activity against antigens expressed at the surface of tumor cells from various malignancies including lymphomas and solid tumors. A component of a CAR is any functional subunit of a CAR that is encoded by an exogenous polynucleotide sequence introduced into the cell. For instance, this component can help to interact with the target antigen, the stability or the localization of the CAR into the cell.

In general, such CAR comprises:
an extracellular ligand binding-domain comprising VH and VL from a monoclonal anti-MUC1 antibody;
a transmembrane domain; and
a signal transducing domain, preferably a cytoplasmic domain comprising a CD3 zeta signalling domain and a co-stimulatory domain.

According to a preferred aspect, the anti-MUC1 chimeric antigen receptor (CAR) of the present invention has an extra cellular ligand binding-domain comprising ScFv having at least 80%, preferably at least 90%, more preferably at least 95%, and even more preferably at least 99% sequence identity with respectively MUC1-A ScFv (SEQ ID NO:17), MUC1-B (SEQ ID NO:27), MUC1-C(SEQ ID NO:37), and/or MUC1-D (SEQ ID NO:47).

Said ScFvs are characterized by a variable light (VL) and heavy (VH) chains comprising specific CDRs, such that the extra cellular ligand binding-domain of the anti-MUC1 CAR of the present invention, as a first example, can comprise:
a variable light (VL) at least 90% identity with SEQ ID NO: 11 (CDR-VL1-A), SEQ ID NO:12 (CDR-VL2-A) and SEQ ID NO:13 (CDR-VL3-A), and
a variable heavy (VH) chain comprising CDRs have respectively at least 90% identity with SEQ ID NO:14 (CDR-VH1-A), SEQ ID NO:15 (CDR-VH2-A) and SEQ ID NO:16 (CDR-VH3-A).
wherein said extra cellular ligand binding-domain preferably comprises VH and VL chains having at least 80%, preferably at least 90%, more preferably at least 95%, and even more preferably at least 99% sequence identity respectively with SEQ ID NO:9 (MUC1-A fullVH) and SEQ ID NO:10 (MUC1-A fullVL).

As a second example, said ScFvs can comprise:
a variable light (VL) chain comprising CDRs that have respectively at least 90% identity with SEQ ID NO:21 (CDR-VL1-B), SEQ ID NO:22 (CDR-VL2-B) and SEQ ID NO:23 (CDR-VL3-B), and
a variable heavy (VH) chain comprising CDRs have respectively at least 90% identity with SEQ ID NO:24 (CDR-VH1-B), SEQ ID NO:25 (CDR-VH2-B) and SEQ ID NO:26 (CDR-VH3-B).
   wherein said extra cellular ligand binding-domain comprises VH and VL chains having at least 80%, preferably at least 90%, more preferably at least 95%, and even more preferably at least 99% sequence identity respectively with SEQ ID NO:19 (MUC1-B fullVH) and SEQ ID NO:20 (MUC1-B fullVL).

As a third example, said ScFvs can comprise:
a variable light (VL) chain comprising CDRs that have respectively at least 90% identity with SEQ ID NO: 31(CDR-VL1-C), SEQ ID NO:32 (CDR-VL2-C) and SEQ ID NO:33 (CDR-VL3-C), and
a variable heavy (VH) chain comprising CDRs have respectively at least 90% identity with SEQ ID NO:34 (CDR-VH1-C), SEQ ID NO:35 (CDR-VH2-C) and SEQ ID NO:36 (CDR-VH3-C).
   wherein said extra cellular ligand binding-domain comprises VH and VL chains having at least 80%, preferably at least 90%, more preferably at least 95%, and even more preferably at least 99% sequence identity respectively with SEQ ID NO:29 (MUC1-C fullVH) and SEQ ID NO:30 (MUC1-C fullVL).

As a fourth example, said ScFvs can comprise:
a variable light (VL) chain comprising CDRs that have respectively at least 90% identity with SEQ ID NO:41 (CDR-VL1-D), SEQ ID NO:42 (CDR-VL2-D) and SEQ ID NO:43 (CDR-VL3-D), and
a variable heavy (VH) chain comprising CDRs have respectively at least 90% identity with SEQ ID NO:44 (CDR-VH1-D), SEQ ID NO:45 (CDR-VH2-D) and SEQ ID NO:46 (CDR-VH3-D).
   wherein said extra cellular ligand binding-domain comprises VH and VL chains having at least 80%, preferably at least 90%, more preferably at least 95%, and even more preferably at least 99% sequence identity respectively with SEQ ID NO:39 (MUC1-D fullVH) and SEQ ID NO:40 (MUC1-D fullVL).

In general, residues in the framework regions of the binding domains can be substituted to improve antigen binding or humanize these regions. These framework substitutions are identified by methods well-known in the art, e.g., by modelling of the interactions of the CDR and framework residues to identify framework residues important for antigen binding and sequence comparison to identify unusual framework residues at particular positions [See, e.g., Queen et al., U.S. Pat. No. 5,585,089; and Riechmann et al., (1988) Nature, 332:323, which are incorporated herein by reference in their entireties].

In some embodiments, VH and VL chains in the above anti-MUC1 CAR constructs can be replaced by humanized versions thereof. For example, and as a preferred embodiment of MUC1-A CARs, VH can be respectively one selected from SEQ ID NO:228 to 234 (humanized variants of MUC1 full VH represented by SEQ ID NO:9) and VL can be selected from SEQ ID NO:235 to 239 (humanized variants of MUC1 full VH represented by SEQ ID NO:10) provided herein in Table 3.

Various embodiments of the invention are provided through the features provided in the claims in view of the common practice and knowledge of one skilled in the art. Detailed sequences of the CAR according to the present invention are detailed in Tables 2, 3, 4, 5, 6 and 7, where each lines or columns should be regarded as independent embodiments of the present invention.

TABLE 2
Amino acid sequence of the different domains,
other than scFvs, constituting the
anti-MUC1 CARs according to the invention.

| Functional | | Raw amino |
|---|---|---|
| domains | SEQ ID # | acid sequence |
| CD8α signal | SEQ ID NO: 2 | MALPVTALLLPLALL |
| peptide | | LHAARP |
| R2 suicide | SEQ ID NO: 3 | SDPGSGGGGSCPYSN |
| switch | | PSLCSGGGGSCPYSN |
| | | PSLCSGGGGS |
| CD8α hinge | SEQ ID NO: 4 | TTTPAPRPPTPAPTI |
| | | ASQPLSLRPEACRPA |
| | | AGGAVHTRGLDFACD |
| CD8α | SEQ ID NO: 5 | IYIWAPLAGTCGVLL |
| transmembrane | | LSLVITLYC |
| domain | | |
| 4-1BB | SEQ ID NO: 6 | KRGRKKLLYIFKQPF |
| co-stimulatory | | MRPVQTTQEEDGCSC |
| domain | | RFPEEEEGGCEL |
| CD3ζ | SEQ ID NO: 7 | RVKFSRSADAPAYQQ |
| signalling | | GQNQLYNELNLGRRE |
| domain | | EYDVLDKRRGRDPEM |
| | | GGKPRRKNPQEGLYN |
| | | ELQKDKMAEAYSEIG |
| | | MKGERRRGKGHDGLY |
| | | QGLSTATKDTYDALH |
| | | MQALPPR |
| Linker | SEQ ID NO. 8 | GGGGSGGGGSGGGGS |

TABLE 3

| | | |
|---|---|---|
| ScFv | | Raw amino |
| sequences | SEQ ID # | acid sequence |

Polypeptide sequences comprised into CLS MUC1-A CAR

| | | |
|---|---|---|
| heavy chain | SEQ ID NO: 9 | QVQLQQSGAELARPGASVKLSCKASGYTFTNYGLSWVKQRT |
| variable region | | GQGLEWIGENHPGSGIIYHNEKFRGKATLTADKSSSTAYVQLS |
| MUC1-A | | SLTSEDSAVYFCARSSGTRGFAYWGQGTLVTVSA |
| light chain | SEQ ID NO: 10 | DVLMTQTPLSLPVSLGDQASISCRSSQSIVHSNGNTYLEWYLQ |
| variable region | | KPGQSPKLLIYKVSNRFSGVPDRFSGSGSGTDFTLKISRVEAE |
| MUC1-A | | DLGVYYCFQGSHGPWTFGGGTKLEIKRA |
| CDR1VH- | SEQ ID NO: 11 | NYGLS |
| MUC1-A | | |
| CDR2VH- | SEQ ID NO: 12 | ENHPGSGIIYHNEKFRG |
| MUC1-A | | |
| CDR3VH- | SEQ ID NO: 13 | SSGTRGFAY |
| MUC1-A | | |
| CDR1VL- | SEQ ID NO: 14 | RSSQSIVHSNGNTYLE |
| MUC1-A | | |
| CDR2VL- | SEQ ID NO: 15 | KVSNRFS |
| MUC1-A | | |
| CDR3VL- | SEQ ID NO: 16 | FQGSHGPWT |
| MUC1-A | | |
| MUC1-A full | SEQ ID NO. 17 | QVQLQQSGAELARPGASVKLSCKASGYTFTNYGLSWVKQRT |
| Binding domain | | GQGLEWIGENHPGSGIIYHNEKFRGKATLTADKSSSTAYVQLS |
| | | SLTSEDSAVYFCARSSGTRGFAYWGQGTLVTVSAGGGGSGG |
| | | GGSGGGGSDVLMTQTPLSLPVSLGDQASISCRSSQSIVHSNG |
| | | NTYLEWYLQKPGQSPKLLIYKVSNRFSGVPDRFSGSGSGTDF |
| | | TLKISRVEAEDLGVYYCFQGSHGPWTFGGGTKLEIKRA |
| CLS MUC1-A | SEQ ID NO. 18 | MALPVTALLLPLALLLHAARPQVQLQQSGAELARPGASVKLSC |
| CAR | | KASGYTFTNYGLSWVKQRTGQGLEWIGENHPGSGIIYHNEKF |
| Full sequence | | RGKATLTADKSSSTAYVQLSSLTSEDSAVYFCARSSGTRGFA |
| | | YWGQGTLVTVSAGGGGSGGGGSGGGGSDVLMTQTPLSLPV |
| | | SLGDQASISCRSSQSIVHSNGNTYLEWYLQKPGQSPKLLIYKV |
| | | SNRFSGVPDRFSGSGSGTDFTLKISRVEAEDLGVYYCFQGSH |
| | | GPWTFGGGTKLEIKRAGSGGGGSCPYSNPSLCSGGGGSCPY |
| | | SNPSLCSGGGGSTTTPAPRPPTPAPTIASQPLSLRPEACRPAA |
| | | GGAVHTRGLDFACDIYIWAPLAGTCGVLLLSLVITLYCKRGRKK |
| | | LLYIFKQPFMRPVQTTQEEDGCSCRFPEEEEGGCELRVKFSR |
| | | SADAPAYQQGQNQLYNELNLGRREEYDVLDKRRGRDPEMG |
| | | GKPRRKNPQEGLYNELQKDKMAEAYSEIGMKGERRRGKGHD |
| | | GLYQGLSTATKDTYDALHMQALPPR |

humanized polypeptide sequences
optionally included into MUC1-A CAR

| | | |
|---|---|---|
| MUC1-A VH | 228 | QVQLVQSGAEVKKPGASVKVSCKASGYTFTNYGLSWVRQAPGQ |
| humanized H1 | | GLEWIGENHPGSGIIYHNQKFQGRVTMTRDTSTSTVYMELSSLRS |
| | | EDTAVYYCARSSGTRGFAYWGQGTLVTVSS |
| MUC1-A VH | 229 | QVQLVQSGAEVKKPGASVKVSCKASGYTFTNYGLSWVRQAPGQ |
| humanized H2 | | GLEWIGENHPGSGIIYHNQKFQGRVTMTADTSTSTVYMELSSLRS |
| | | EDTAVYYCARSSGTRGFAYWGQGTLVTVSS |
| MUC1-A VH | 230 | QVQLVQSGAEVKKPGASVKVSCKASGYTFTNYGLSWVRQAPGQ |
| humanized H3 | | GLEWIGENHPGSGIIYHNQKFQGRVTMTRDKSTSTVYMELSSLR |
| | | SEDTAVYYCARSSGTRGFAYWGQGTLVTVSS |
| MUC1-A VH | 231 | QVQLVQSGAEVKKPGASVKVSCKASGYTFTNYGLSWVRQAPGQ |
| humanized H4 | | GLEWIGENHPGSGIIYHNQKFQGRVTMTADKSTSTVYMELSSLR |
| | | SEDTAVYYCARSSGTRGFAYWGQGTLVTVSS |
| MUC1-A VH | 232 | QVQLVQSGAEVKKPGASVKVSCKASGYTFTNYGLSWVRQAPGQ |
| humanized H5 | | GLEWIGENHPGSGIIYHNEKFRGRVTMTADTSTSTVYMELSSLRS |
| | | EDTAVYYCARSSGTRGFAYWGQGTLVTVSS |
| MUC1-A VH | 233 | QVQLVQSGAEVKKPGASVKVSCKASGYTFTNYGLSWVRQAPGQ |
| humanized H6 | | GLEWIGENHPGSGIIYHNEKFRGRVTMTRDKSTSTVYMELSSLRS |
| | | EDTAVYYCARSSGTRGFAYWGQGTLVTVSS |
| MUC1-A VH | 234 | QVQLVQSGAEVKKPGASVKVSCKASGYTFTNYGLSWVRQAPGQ |
| humanized H7 | | GLEWIGENHPGSGIIYHNEKFRGRVTMTADKSTSTVYMELSSLRS |
| | | EDTAVYYCARSSGTRGFAYWGQGTLVTVSS |
| MUC1-A VL | 235 | DVVMTQSPLSLPVTLGQPASISCRSSQSIVHSNGNTYLEWFQQR |
| humanized L1 | | PGQSPRRLIYKVSNRFSGVPDRFSGSGSGTDFTLKISRVEAEDVG |
| | | VYYCFQGSHGPWTFGQGTKVEIKRA |
| MUC1-A VL | 236 | DVVMTQSPLSLPVTLGQPASISCRSSQSIVHSNGNTYLEWYQQR |
| humanized L2 | | PGQSPRRLIYKVSNRFSGVPDRFSGSGSGTDFTLKISRVEAEDVG |
| | | VYYCFQGSHGPWTFGQGTKVEIKRA |
| MUC1-A VL | 237 | DVVMTQSPLSLPVTLGQPASISCRSSQSIVHSNGNTYLEWFQQR |
| humanized L3 | | PGQSPRLLIYKVSNRFSGVPDRFSGSGSGTDFTLKISRVEAEDVG |
| | | VYYCFQGSHGPWTFGQGTKVEIKRA |
| MUC1-A VL | 238 | DVVMTQSPLSLPVTLGQPASISCRSSQSIVHSNGNTYLEWYQQR |
| humanized L4 | | PGQSPRLLIYKVSNRFSGVPDRFSGSGSGTDFTLKISRVEAEDVG |
| | | VYYCFQGSHGPWTFGQGTKVEIKRA |
| MUC1-A VL | 239 | DVLMTQSPLSLPVTLGQPASISCRSSQSIVHSNGNTYLEWYQQR |
| humanized L5 | | PGQSPRLLIYKVSNRFSGVPDRFSGSGSGTDFTLKISRVEAEDVG |
| | | VYYCFQGSHGPWTFGQGTKVEIKRA |

TABLE 4
Polypeptide sequences
comprised into CLS MUC1-B CAR

| ScFv | SEQ ID | Raw amino |
|---|---|---|
| sequences | # | acid sequence |
| heavy chain | SEQ ID NO: 19 | EVQLQQSGGGLVQPGGSMK |
| variable region | | LSCVASGFTFSNYWMNWVR |
| MUC1-B | | QSPEKGLEWVAEIRLKSNN |
| | | YATHYAESVKGRFTISRDD |
| | | SKSSVYLQMNNLRAEDTGI |
| | | YYCTFGNSFAYWGQGTTVT |
| | | VSS |
| light chain | SEQ ID NO: 20 | DIVVTQESALTTSPGETVT |
| variable region | | LTCRSSTGAVTTSNYANWV |
| MUC1-B | | QEKPDHLFTGLIGGTNNRA |
| | | PGVPARFSGSLIGDKAALT |
| | | ITGAQTEDEAIYFCALWYS |
| | | NHWVFGGGTKLTVL |
| CDR1VH- | SEQ ID NO: 21 | GFTFSNYWMN |
| MUC1-B | | |
| CDR2VH- | SEQ ID NO: 22 | EIRLKSNNYATHYAESVKG |
| MUC1-B | | |
| CDR3VH- | SEQ ID NO: 23 | GNSFAY |
| MUC1-B | | |
| CDR1VL- | SEQ ID NO: 24 | TGAVTTSNYAN |
| MUC1-B | | |
| CDR2VL- | SEQ ID NO: 25 | GGTNNRAP |
| MUC1-B | | |
| CDR3VL- | SEQ ID NO: 26 | ALWYSNHWV |
| MUC1-B | | |
| MUC1-B full | SEQ ID NO. 27 | EVQLQQSGGGLVQPGGSMK |
| Binding domain | | LSCVASGFTFSNYWMNWVR |
| | | QSPEKGLEWVAEIRLKSNN |
| | | YATHYAESVKGRFTISRDD |
| | | SKSSVYLQMNNLRAEDTGI |
| | | YYCTFGNSFAYWGQGTTVT |
| | | VSSGGGGSGGGGSGGGGSD |
| | | IVVTQESALTTSPGETVTL |
| | | TCRSSTGAVTTSNYANWVQ |
| | | EKPDHLFTGLIGGTNNRAP |
| | | GVPARFSGSLIGDKAALTI |
| | | TGAQTEDEAIYFCALWYSN |
| | | HWVFGGGTKLTVL |
| CLS MUC1-B | SEQ ID NO. 28 | MALPVTALLLPLALLLHAA |
| CAR Full | | RPEVQLQQSGGGLVQPGGS |
| sequence | | MKLSCVASGFTFSNYWMNW |
| | | VRQSPEKGLEWVAEIRLKS |
| | | NNYATHYAESVKGRFTISR |
| | | DDSKSSVYLQMNNLRAEDT |
| | | GIYYCTFGNSFAYWGQGTT |
| | | VTVSSGGGGSGGGGSGGGG |
| | | SDIVVTQESALTTSPGETV |
| | | TLTCRSSTGAVTTSNYANW |
| | | VQEKPDHLFTGLIGGTNNR |
| | | APGVPARFSGSLIGDKAAL |
| | | TITGAQTEDEAIYFCALWY |
| | | SNHWVFGGGTKLTVLGSGG |
| | | GGSCPYSNPSLCSGGGGSC |
| | | PYSNPSLCSGGGGSTTTPA |
| | | PRPPTPAPTIASQPLSLRP |
| | | EACRPAAGGAVHTRGLDFA |
| | | CDIYIWAPLAGTCGVLLLS |
| | | LVITLYCKRGRKKLLYIFK |
| | | QPFMRPVQTTQEEDGCSCR |
| | | FPEEEEGGCELRVKFSRSA |
| | | DAPAYQQGQNQLYNELNLG |
| | | RREEYDVLDKRRGRDPEMG |
| | | GKPRRKNPQEGLYNELQKD |
| | | KMAEAYSEIGMKGERRRGK |
| | | GHDGLYQGLSTATKDTYDA |
| | | LHMQALPPR |

TABLE 5
Polypeptide sequences
comprised into CLSMUC1-C CAR

| ScFv | SEQ ID | Raw amino |
|---|---|---|
| sequences | # | acid sequence |
| heavy chain | SEQ ID NO: 29 | QMQLVQSEAELKKPGASVKV |
| variable region | | SCKASGYSFTGHYMHWVRQA |
| MUC1-C | | PGQGLEWMGWIDPVTGGTKY |
| | | AQNFQGWVTMTRDTSIRTAY |
| | | LELSRLRSDDTAMYYCAREV |
| | | TGDRGQFDKWGQGTLVTVAS |
| light chain | SEQ ID NO: 30 | QSVLTQPPSVSVAPGKTARI |
| variable region | | TCGGNNIGSKSVHWYQQKPG |
| MUC1-C | | QAPALVIYYGSNRPSGIPER |
| | | FSGSNSGNTATLTISRVEAG |
| | | DEADYYCQVWDSSSDWVFGG |
| | | GTKLTVL |
| CDR1VH- | SEQ ID NO: 31 | GYSFTGHY |
| MUC1-C | | |
| CDR2VH- | SEQ ID NO: 32 | IDPVTGGT |
| MUC1-C | | |
| CDR3VH- | SEQ ID NO: 33 | AREVTGDRGQ |
| MUC1-C | | |
| CDR1VL- | SEQ ID NO: 34 | NIGSKS |
| MUC1-C | | |
| CDR2VL- | SEQ ID NO: 35 | YGS |
| MUC1-C | | |
| CDR3VL- | SEQ ID NO: 36 | QVWDSSSDWV |
| MUC1-C | | |
| MUC1-C full | SEQ ID NO. 37 | QMQLVQSEAELKKPGASVKV |
| Binding domain | | SCKASGYSFTGHYMHWVRQA |
| | | PGQGLEWMGWIDPVTGGTKY |
| | | AQNFQGWVTMTRDTSIRTAY |
| | | LELSRLRSDDTAMYYCAREV |
| | | TGDRGQFDKWGQGTLVTVAS |
| | | GGGGSGGGGSGGGGSQSVLT |
| | | QPPSVSVAPGKTARITCGGN |
| | | NIGSKSVHWYQQKPGQAPAL |
| | | VIYYGSNRPSGIPERFSGSN |
| | | SGNTATLTISRVEAGDEADY |
| | | YCQVWDSSSDWVFGGGTKLT |
| | | VL |
| CLS MUC1-C | SEQ ID NO. 38 | MALPVTALLLPLALLLHAAR |
| CAR-Full | | PQMQLVQSEAELKKPGASVK |
| sequence | | VSCKASGYSFTGHYMHWVRQ |
| | | APGQGLEWMGWIDPVTGGTK |
| | | YAQNFQGWVTMTRDTSIRTA |
| | | YLELSRLRSDDTAMYYCARE |
| | | VTGDRGQFDKWGQGTLVTVA |
| | | SGGGGSGGGGSGGGGSQSVL |
| | | TQPPSVSVAPGKTARITCGG |
| | | NNIGSKSVHWYQQKPGQAPA |
| | | LVIYYGSNRPSGIPERFSGS |
| | | NSGNTATLTISRVEAGDEAD |
| | | YYCQVWDSSSDWVFGGGTKL |
| | | TVLGSGGGGSCPYSNPSLCS |
| | | GGGGSCPYSNPSLCSGGGGS |
| | | TTTPAPRPPTPAPTIASQPL |
| | | SLRPEACRPAAGGAVHTRGL |
| | | DFACDIYIWAPLAGTCGVLL |
| | | LSLVITLYCKRGRKKLLYIF |
| | | KQPFMRPVQTTQEEDGCSCR |
| | | FPEEEEGGCELRVKFSRSAD |
| | | APAYQQGQNQLYNELNLGRR |
| | | EEYDVLDKRRGRDPEMGGKP |
| | | RRKNPQEGLYNELQKDKMAE |
| | | AYSEIGMKGERRRGKGHDGL |
| | | YQGLSTATKDTYDALHMQAL |
| | | PPR |

TABLE 6
Polypeptide sequences
comprised into CLS MUC1-D CAR

| ScFv | SEQ ID | Raw amino |
|---|---|---|
| sequences | # | acid sequence |
| heavy chain | SEQ ID NO: 39 | QVQLQQSDAELVKPGSSVKI |
| variable region | | SCKASGYTFTDHAIHWVKQK |
| MUC1-D | | PEQGLEWIGHFSPGNTDIKY |
| | | NDKFKGKATLTVDRSSSTAY |
| | | MQLNSLTSEDSAVYFCKTST |
| | | FFFDYWGQGTTLTVSS |
| light chain | SEQ ID NO: 40 | DIVMTQSPSSLTVTAGEKVT |
| variable region | | MICKSSQSLLNSGDQKNYLT |
| MUC1-D | | WYQQKPGQPPKLLIFWASTR |
| | | ESGVPDRFTGSGSGTDFTLT |
| | | ISSVQAEDLAVYYCQNDYSY |
| | | PLTFGAGTKLELK |
| CDR1VH- | SEQ ID NO: 41 | YTFTDHAIH |
| MUC1-D | | |
| CDR2VH- | SEQ ID NO: 42 | WIGHFSPGNTDIKY |
| MUC1-D | | |
| CDR3VH- | SEQ ID NO: 43 | KTSTFFFDY |
| MUC1-D | | |
| CDR1VL- | SEQ ID NO: 44 | QSLLNSGDQKNYLT |
| MUC1-D | | |
| CDR2VL- | SEQ ID NO: 45 | LLIFWASTRES |
| MUC1-D | | |
| CDR3VL- | SEQ ID NO: 46 | QNDYSYPL |
| MUC1-D | | |
| MUC1-D | SEQ ID NO. 47 | QVQLQQSDAELVKPGSSVKI |
| full Binding | | SCKASGYTFTDHAIHWVKQK |
| domain | | PEQGLEWIGHFSPGNTDIKY |
| | | NDKFKGKATLTVDRSSSTAY |
| | | MQLNSLTSEDSAVYFCKTST |
| | | FFFDYWGQGTTLTVSSGGGG |
| | | SGGGGSGGGGSDIVMTQSPS |
| | | SLTVTAGEKVTMICKSSQSL |
| | | LNSGDQKNYLTWYQQKPGQP |
| | | PKLLIFWASTRESGVPDRFT |
| | | GSGSGTDFTLTISSVQAEDL |
| | | AVYYCQNDYSYPLTFGAGTK |
| | | LELK |
| CLS MUC1-D | SEQ ID NO. 48 | MALPVTALLLPLALLLHAAR |
| CAR-Full | | PQVQLQQSDAELVKPGSSVK |
| sequence | | ISCKASGYTFTDHAIHWVKQ |
| | | KPEQGLEWIGHFSPGNTDIK |
| | | YNDKFKGKATLTVDRSSSTA |
| | | YMQLNSLTSEDSAVYFCKTS |
| | | TFFFDYWGQGTTLTVSSGGG |
| | | GSGGGGSGGGGSDIVMTQSP |
| | | SSLTVTAGEKVTMICKSSQS |
| | | LLNSGDQKNYLTWYQQKPGQ |
| | | PPKLLIFWASTRESGVPDRF |
| | | TGSGSGTDFTLTISSVQAED |
| | | LAVYYCQNDYSYPLTFGAGT |
| | | KLELKGSGGGGSCPYSNPSL |
| | | CSGGGGSCPYSNPSLCSGGG |
| | | GSTTTPAPRPPTPAPTIASQ |
| | | PLSLRPEACRPAAGGAVHTR |
| | | GLDFACDIYIWAPLAGTCGV |
| | | LLLSLVITLYCKRGRKKLLY |
| | | IFKQPFMRPVQTTQEEDGCS |
| | | CRFPEEEEGGCELRVKFSRS |
| | | ADAPAYQQGQNQLYNELNLG |
| | | RREEYDVLDKRRGRDPEMGG |
| | | KPRRKNPQEGLYNELQKDKM |
| | | AEAYSEIGMKGERRRGKGHD |
| | | GLYQGLSTATKDTYDALHMQ |
| | | ALPPR |

TABLE 7
Polypeptide structure of MUC1-A, MUC1-B, MUC1-C and MUC1-D CARs

| CAR | Domains | | | | | | | |
|---|---|---|---|---|---|---|---|---|
| Designa- | signal | | | | | | | |
| tion | peptide | | | | CD8α | CD8α | 4-1BB | CD3ζ |
| Full | (optional) | VH | linker | VL | hinge | TM | IC | IC |
| CAR | SEQ ID | SEQ ID NO: 9 | SEQ ID | SEQ ID | SEQ ID | SEQ ID | SEQ ID | SEQ ID |
| MUC1-A | NO: 2 | (or humanized | NO: 8 | NO: 10 | NO: 4 | NO: 5 | NO: 6 | NO: 7 |
| (SEQ ID | | versions | | (or humanized | | | | |
| NO: 18) | | SEQ ID NO: | | version | | | | |
| | | 228 to 234) | | SEQ ID NO: | | | | |
| | | | | 235 to 239) | | | | |
| CAR | SEQ ID | SEQ ID | SEQ ID | SEQ ID | SEQ ID | SEQ ID | SEQ ID | SEQ ID |
| MUC1-B | NO: 2 | NO: 19 | NO: 8 | NO: 20 | NO: 4 | NO: 5 | NO: 6 | NO: 7 |
| (SEQ ID | | | | | | | | |
| NO: 28) | | | | | | | | |
| CAR | SEQ ID | SEQ ID | SEQ ID | SEQ ID | SEQ ID | SEQ ID | SEQ ID | SEQ ID |
| MUC1-C | NO: 2 | NO: 29 | NO: 8 | NO: 30 | NO: 4 | NO: 5 | NO: 6 | NO: 7 |
| (SEQ ID | | | | | | | | |
| NO: 38) | | | | | | | | |
| CAR | SEQ ID | SEQ ID | SEQ ID | SEQ ID | SEQ ID | SEQ ID | SEQ ID | SEQ ID |
| MUC1-D | NO: 2 | NO: 39 | NO : 8 | NO: 40 | NO: 4 | NO: 5 | NO: 6 | NO: 7 |
| (SEQ ID | | | | | | | | |
| NO: 48) | | | | | | | | |

The signal transducing domain or intracellular signaling domain of a CAR according to the present invention is responsible for intracellular signaling following the binding of extracellular ligand binding domain to the target resulting in the activation of the immune cell and immune response. In other words, the signal transducing domain is responsible for the activation of at least one of the normal effector functions of the immune cell in which the CAR is expressed. For example, the effector function of a T cell can be a cytolytic activity or helper activity including the secretion of cytokines. Thus, the term “signal transducing domain” refers to the portion of a protein which transduces the effector signal function signal and directs the cell to perform a specialized function.

Preferred examples of signal transducing domain for use in a CAR can be the cytoplasmic sequences of the T cell receptor and co-receptors that act in concert to initiate signal transduction following antigen receptor engagement, as well as any derivate or variant of these sequences and any synthetic sequence that has the same functional capability. Signal transduction domain comprises two distinct classes of cytoplasmic signaling sequence, those that initiate antigen-dependent primary activation, and those that act in an antigen-independent manner to provide a secondary or co-stimulatory signal. Primary cytoplasmic signaling sequence can comprise signaling motifs which are known as immunoreceptor tyrosine-based activation motifs of ITAMs. ITAMs are well defined signaling motifs found in the intracytoplasmic tail of a variety of receptors that serve as binding sites for syk/zap70 class tyrosine kinases. Examples of ITAM used in the invention can include as non-limiting examples those derived from TCRzeta, FcRgamma, FcRbeta, FcRepsilon, CD3gamma, CD3delta, CD3epsilon, CD5, CD22, CD79a, CD79b and CD66d. In a preferred embodiment, the signaling transducing domain of the CAR can comprise the CD3zeta signaling domain which has amino acid sequence with at least 70%, preferably at least 80%, more preferably at least 90%, 95% 97% or 99% sequence identity with amino acid sequence selected from the group consisting of (SEQ ID NO:7).

In particular embodiment the signal transduction domain of the CAR of the present invention comprises a co-stimulatory signal molecule. A co-stimulatory molecule is a cell surface molecule other than an antigen receptor or their ligands that is required for an efficient immune response. “Co-stimulatory ligand” refers to a molecule on an antigen presenting cell that specifically binds a cognate co-stimulatory molecule on a T-cell, thereby providing a signal which, in addition to the primary signal provided by, for instance, binding of a TCR/CD3 complex with an MHC molecule loaded with peptide, mediates a T cell response, including, but not limited to, proliferation activation, differentiation and the like. A co-stimulatory ligand can include but is not limited to CD7, B7-1 (CD80), B7-2 (CD86), PD-L1, PD-L2, 4-1BBL, OX40L, inducible costimulatory ligand (ICOS-L), intercellular adhesion molecule (ICAM, CD30L, CD40, CD70, CD83, HLA-G, MICA, M1CB, HVEM, lymphotoxin beta receptor, 3/TR6, ILT3, ILT4, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as but not limited to, CD27, CD28, 4-1EE, OX40, CD30, CD40, PD-1, ICOS, lymphocyte function-associated antigen-1 (LFA-1), CD2, CD7, LTGHT, NKG2C, B7-H3, a ligand that specifically binds with CD83.

In a preferred embodiment, the signal transduction domain of the CAR of the present invention comprises a part of co-stimulatory signal molecule selected from the group consisting of fragment of 4-1BE (GenBank: AAA53133.) and CD28 (NP_006130.1). In particular the signal transduction domain of the CAR of the present invention comprises amino acid sequence which comprises at least 70%, preferably at least 80%, more preferably at least 90%, 95% 97% or 99% sequence identity with either 4-1BE or CD28. Accordingly, the anti-MUC1 chimeric antigen receptor as per the present invention preferably comprises a CD3 zeta signalling domain that has at least 80% identity with SEQ ID NO:7 and generally comprises a co-stimulatory domain that has at least 80% identity with SEQ ID NO:6. (4-11BE).

A CAR according to the present invention is generally expressed on the surface membrane of the cell. Thus, such CAR further comprises a transmembrane domain. The distinguishing features of appropriate transmembrane domains comprise the ability to be expressed at the surface of a cell, preferably in the present invention an immune cell, in particular lymphocyte cells or Natural killer (NK) cells, and to interact together for directing cellular response of immune cell against a predefined target cell. The transmembrane domain can be derived either from a natural or from a synthetic source. The transmembrane domain can be derived from any membrane-bound or transmembrane protein. As non-limiting examples, the transmembrane polypeptide can be a subunit of the T-cell receptor such as α, β, γ or ζ, polypeptide constituting CD3 complex, IL2 receptor p55 (α chain), p75 (β chain) or γ chain, subunit chain of Fc receptors, in particular Fcγ receptor III or CD proteins. Alternatively the transmembrane domain can be synthetic and can comprise predominantly hydrophobic residues such as leucine and valine. In a preferred embodiment said transmembrane domain is derived from the human CD8 alpha chain (e.g. NP_001139345.1) The transmembrane domain can further comprise a hinge region between said extracellular ligand-binding domain and said transmembrane domain. The term “hinge region” used herein generally means any oligo- or polypeptide that functions to link the transmembrane domain to the extracellular ligand-binding domain. In particular, hinge region are used to provide more flexibility and accessibility for the extracellular ligand-binding domain. A hinge region may comprise up to 300 amino acids, preferably 10 to 100 amino acids and most preferably 25 to 50 amino acids. Hinge region may be derived from all or part of naturally occurring molecules, such as from all or part of the extracellular region of CD8, CD4 or CD28, or from all or part of an antibody constant region. Alternatively the hinge region may be a synthetic sequence that corresponds to a naturally occurring hinge sequence, or may be an entirely synthetic hinge sequence. In a preferred embodiment said hinge domain comprises a part of human CD8 alpha chain, FcγRIIIα receptor or IgG1 respectively, or hinge polypeptides which display preferably at least 80%, more preferably at least 90%, 95% 97% or 99% sequence identity with these polypeptides.

Accordingly a chimeric antigen receptor (CAR) according to the present invention comprises a hinge between the extracellular ligand-binding domain and the transmembrane domain, said hinge being generally selected from CD8α hinge, IgG1 hinge and FcγRIIIα hinge or polypeptides sharing at least 80%, preferably at least 90%, more preferably at least 95%, and even more preferably at least 99% sequence identity with these polypeptides, in particular with SEQ ID NO:4 (CD8α).

A CAR according to the invention generally further comprises a transmembrane Domain™ preferably selected from CD8α and 4-11E1, more preferably from CD8α-TM or a polypeptide showing at least 80%, more preferably at least 90%, 95% 97% or 99% sequence identity with SEQ ID NO:5 (CD8α TM).

According to a further embodiment, the anti-MUC1 CAR according to the invention comprises a safety switch useful for sorting, purifying and/or depleting the engineered immune cells. Although the methods of the invention are designed to be performed ex-vivo, depletion may be performed in-vivo to control immune cell's expansion into the patient and to potentially stop the effects of the treatment by using antibodies approved for human therapeutic use by regulatory agencies. Examples of mAb-specific epitopes (and their corresponding mAbs) that can be integrated in the extracellular binding domain of the CAR of the invention are listed in Table 8.

TABLE 8
Examples of mAb-specific epitopes
(and their corresponding mAbs), which can be
inserted in the extracellular binding
domain of the CAR of the invention.
Rituximab

| | | |
|---|---|---|
| Mimotope | SEQ ID NO: 49 | CPYSNPSLC |

Palivizumab

| | | |
|---|---|---|
| Epitope | SEQ ID NO: 50 | NSELLSLINDMPITNDQKKLMSNN |

Cetuximab

| | | |
|---|---|---|
| Mimotope 1 | SEQ ID NO: 51 | CQFDLSTRRLKC |
| Mimotope 2 | SEQ ID NO: 52 | CQYNLSSRALKC |
| Mimotope 3 | SEQ ID NO: 53 | CVWQRWQKSYVC |
| Mimotope 4 | SEQ ID NO: 54 | CMWDRFSRWYKC |

Nivolumab

| | | |
|---|---|---|
| Epitope 1 | SEQ ID NO: 55 | SFVLNWYRMSPSNQTDKLAAFPEDR |
| Epitope 2 | SEQ ID NO: 56 | SGTYLCGAISLAPKAQIKE |

QBEND-10

| | | |
|---|---|---|
| Epitope | SEQ ID NO: 57 | ELPTQGTFSNVSTNVSPAKPTTTA |

Alemtuzumab

| | | |
|---|---|---|
| Epitope | SEQ ID NO: 58 | GQNDTSQTSSPS |

Accordingly, a specific CAR according to the invention can comprise a safety switch which comprises at least one exogenous mAb epitope listed in Table 8, preferably, a safety switch comprising the epitope CPYSNPSLC (SEQ ID NO:49) that is specifically bound by rituximab. More preferably, such a CAR comprises a safety switch referred to as “R2”, that has at least 90% identity with SEQ ID NO:3.

The structure of the preferred polypeptide structure of the anti-MUC1 CARs of the present invention are illustrated in Table 7.

A CAR according to the invention can also comprises a signal peptide to help its expression at the surface of the engineered cells. The chimeric antigen receptor (CAR) generally form single-chain polypeptides, but may also be produced in multi-chain formats as described for instance in WO2014039523.

Accordingly, an anti-MUC1 CAR according to the invention can present a transmembrane domain that shares at least 80%, preferably at least 90%, more preferably at least 95%, and even more preferably at least 99% sequence identity with SEQ ID NO:5 from CD8α.

An anti-MUC1 CAR according to the invention can further comprise a hinge between the extracellular ligand-binding domain and the transmembrane domain, which is preferably selected from CD8α hinge, IgG1 hinge and FcγRIIIα hinge. In a preferred embodiment, the hinge shares at least 80%, preferably at least 90%, more preferably at least 95%, and even more preferably at least 99% sequence identity, respectively with SEQ ID NO:4 (CD8α).

In preferred embodiments, the anti-MUC1CAR has a polypeptide structure comprising an CD8α hinge having at least 80% identity with the amino acid sequence set forth in SEQ ID NO:4 together with a CD8α transmembrane domain having at least 80% identity with the amino acid sequence set forth in SEQ ID NO:5.

In preferred embodiments, the anti-MUC1CAR of the present invention comprises a safety switch, which is a polypeptide sequence specifically recognized by an antibody suitable for use in therapy, such as an epitope selected from Table 8. For instance, a safety switch comprising the epitope CPYSNPSLC (SEQ ID NO:49) can be specifically bound by rituximab (Rituxan, Hoffmann-La Roche), which is an approved anti-CD20 chimeric monoclonal antibody commonly used in cancer treatment to deplete B-cells.

In preferred embodiments, the anti-MUC1CAR of the present invention comprises a co-stimulatory domain from 4-1BB or CD28, preferably 4-1BB or any functional similar domain that has at least 80% identity with SEQ ID NO:6.

In preferred embodiments, the anti-MUC1CAR of the present invention comprises a CD3 zeta signalling domain that has at least 80% identity with SEQ ID NO:7. It can also comprise a signal peptide to be better addresses at the cell surface.

As illustrated in the examples and Tables 2 to 8, preferred CARs according to the invention are the anti-MUC1 CAR, which have respectively at least 80%, preferably at least 90%, more preferably at least 95%, and even more preferably at least 99% overall amino acid sequence identity with SEQ ID NO:18 (CLS MUC1-A), SEQ ID NO:28 (CLS MUC1-B), SEQ ID NO:38 (CLS MUC1-C) and SEQ ID NO:48 (CLS MUC1-D). One preferred anti MUC1-CAR is CLS MUC1-A that is represented by SEQ ID NO:18.

More generally, the CARs of the present invention are produced by assembling the different polynucleotides sequences encoding the successive fragments of the CAR polypeptide(s) into vectors for transfection and expression into immune cells as described in the art and as reviewed for instance by [Boyiadzis, M. M., et al. (2018) Chimeric antigen receptor (CAR) T therapies for the treatment of hematologic malignancies: clinical perspective and significance. j. immunotherapy cancer 6, 137].

The present invention is drawn to the polynucleotides and vectors as well as any intermediary products intervening in any steps of the process of manufacturing of the immune cells referred herein.
By “vector” is meant a nucleic acid molecule capable of transporting another nucleic acid to which it has been linked. A “vector” in the present invention includes, but is not limited to, a viral vector, a plasmid, a RNA vector or a linear or circular DNA or RNA molecule which may consists of a chromosomal, non-chromosomal, semi-synthetic or synthetic nucleic acids. Preferred vectors are those capable of autonomous replication (episomal vector) and/or expression of nucleic acids to which they are linked (expression vectors). Large numbers of suitable vectors are known to those of skill in the art and commercially available. Viral vectors include retrovirus, adenovirus, parvovirus (e.g. adeno-associated viruses (AAV), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996).

In particular, the present invention provides inter alia vectors in the form of non-integrative lentiviral vector (IDLV) or AAV vector comprising an exogenous polynucleotide sequence as described herein encoding a CAR, IL-12, dnTGFβR, HLA-E or any other useful transgene, for their use as donor polynucleotide templates to perform gene targeted integration.

Such lentiviral vector may comprise a polynucleotide sequence encoding a CAR according to the present invention operably linked to a promoter (such as the Spleen Focus Forming Virus promoter (SFFV)). By “operably linked” it is meant a juxtaposition wherein the components described are in a relationship permitting them to function in their intended manner. A gene (such as a CAR encoding polynucleotide sequence) is “operably linked” to a promoter when its transcription is under the control of said promoter and this transcription results in the production of the product encoded by said gene.

The lentiviral vector of the present invention typically contains regulatory elements such as 5′ and 3′ long terminal repeat (LTR) sequences, but may also contain other structural and functional genetic elements that are primarily derived from a lentivirus. Such structural and functional genetic elements are well known in the art. The lentiviral vector may, for example, contain the genes gag, pol and env. Preferably, however, the lentiviral vector of the present invention does not contain the genes gag, pol and env. As further regulatory elements the lentiviral vector may include one or more (such as two or more) of a packaging signal (such as the packaging signal tp), a primer binding site, a trans-activation-responsive region (TAR) and a rev-responsive element (RRE).

The 5′ and 3′ long terminal repeat (LTR) sequences typically flanking the lentiviral genome have promoter/enhancer activity and are essential for the correct expression of the full-length lentiviral vector transcript. The LTRs usually include the repetitive sequence U3RU5 present at both the 5′- and 3′ ends of a double-stranded DNA molecule, which is a combination of 5′ R-U5 segment and the 3′ U3-R segment of the single-stranded RNA, wherein repetition R occurs at both termini of the RNA, while U5 (unique sequence 5) only occurs at the 5′ end of the RNA and U3 (unique sequence 3) only occurs at the 3′ end of the RNA. Lentiviral vectors can been improved in their safety by removal of the U3 sequence, resulting in “self-inactivating” vectors that are entirely devoid of viral promoter and enhancer sequences originally present within the LTRs. Consequently, the vector is capable of infecting and then integrating into the host genome only once, and cannot be passed further, thereby increasing the safety of the use of the vector as a gene delivery vector.

According to some embodiments, the lentiviral vector is a self-inactivating (SIN) lentiviral vector. According to particular embodiments, the lentiviral vector contains a 3′ LTR in which the 3′ LTR enhancer-promoter sequence (i.e. U3 sequence) has been modified (e.g., deleted).

According to some embodiments, the lentiviral vector comprises a polynucleotide sequence which comprises one or several of the following elements in a 5′ to 3′ order:
a 5′ long terminal repeat (5′ LTR);
a promoter (such as the EF1-alpha promoter);
a polynucleotide sequence encoding a chimeric antigen receptor according to the present invention; and/or
a 3′ long terminal repeat (3′ LTR), preferably 3′ self-inactivating LTR.

According to particular embodiments, the lentiviral vector can further comprise a polynucleotide sequence which comprises at least one of the following elements in a 5′ to 3′ order:
a 5′ long terminal repeat (5′ LTR);
a promoter (such as the EF1-alpha promoter);
a CAR according to the present invention optionally comprising a safety switch, such as R2,
a polynucleotide sequence encoding a 2A peptide;
a polynucleotide sequence encoding a or any further polypeptide to be co-expressed with the CAR, such as dnTGFβR;
and/or
a 3′ long terminal repeat (3′ LTR), preferably 3′ self-inactivating LTR.

Alternatively, the lentiviral vector can comprise at least one of the following elements in a 5′ to 3′ order:
a 5′ long terminal repeat (5′ LTR);
a promoter (such as the EF1-alpha promoter);
a polynucleotide sequence encoding a or any further polypeptide to be co-expressed with the CAR, such as dnTGFβR;
a polynucleotide sequence encoding a 2A peptide;
a CAR according to the present invention optionally comprising a safety switch, such as R2, and/or
a 3′ long terminal repeat (3′ LTR), preferably 3′ self-inactivating LTR.

In general, the resulting vector form a single transcription unit operably linked to the promoter of item (b) and are all transcribed under the control of said promoter.

AAV vectors, especially vectors from the AAV6 family [Wang, J., et al. (2015) Homology-driven genome editing in hematopoietic stem and progenitor cells using ZFN mRNA and AAV6 donors. Nat Biotechnol 33, 1256-1263] are particularly useful to introduce the anti-MUC1CARs according to the present invention into the genome by using site-specific homologous recombination. In general, site specific homologous recombination is induced in immune cells by expressing rare-cutting endonucleases, such as TALEN, as already taught in EP3276000 and WO2018073391 with respect to other CARs for treating blood cancers. CAR site-specific integration can have several benefits, such as a more stable integration, an integration that places the transgene under the transcription control of an endogenous promoter at a selected locus, an integration that can inactivate an endogenous locus. These later aspects are detailed into the following section pertaining to the genome engineering of the therapeutic immune cells.

As one object of the present invention is provided an AAV vector comprising a polynucleotide sequence encoding an anti-MUC1 CAR as previously specified and optionally another sequence encoding a cis-regulatory elements (e.g. 2A peptide cleavage site) or an internal ribosome entry site (IRES), allowing the co-expression of a third sequence encoding a product improving the therapeutic potency of the engineered immune cells. Example is given herein of over expression of dnTGFβRII, which was found to reduce SMAD2-3 phosphorylation, which concurs to reduce exhaustion of the cells that is induced by TGFβ in the tumor environment.

The term “therapeutic properties” encompasses the different ways such cells can be improved in the perspective of their use in therapeutic treatments. This means that the cells are genetically engineered to confer them a therapeutic advantage benefit (i.e. therapeutic potency) or to facilitate their use or their production. For instance, the genetic engineering can concur to the effector cells having better survival, faster growth, shorter cell cycles, improved immune activity, be more functional, more differentiated, more specific with respect to their target cells, more sensitive or resistant to drugs, less sensitive to glucose deprivation, oxygen or amino acid depletion (i.e. resilient to tumor microenvironment). Progenitor cells may be more productive, better tolerated by the recipient patient, more likely to produce cells that will differentiate in the desired effector cells. These examples of “therapeutic properties” are given as examples without limitation.

### Genome Engineering of the Anti-MUC1 CAR Immune Cells for Cell Therapy

By “immune cell” is meant a cell of hematopoietic origin functionally involved in the initiation and/or execution of innate and/or adaptive immune response, such as typically CD3 or CD4 positive cells. The immune cell according to the present invention may be a dendritic cell, killer dendritic cell, a mast cell, a NK-cell, a B-cell or a T-cell selected from the group consisting of inflammatory T-lymphocytes, cytotoxic T-lymphocytes, regulatory T-lymphocytes or helper T-lymphocytes.

By “primary cell” or “primary cells” are intended cells taken directly from living tissue (e.g. biopsy material) and established for growth in vitro for a limited amount of time, meaning that they can undergo a limited number of population doublings. Primary cells are opposed to continuous tumorigenic or artificially immortalized cell lines. Non-limiting examples of such cell lines are CHO-K1 cells; HEK293 cells; Caco2 cells; U2-OS cells; NIH 3T3 cells; NSO cells; SP2 cells; CHO-S cells; DG44 cells; K-562 cells, U-937 cells; MRC5 cells; IMR90 cells; Jurkat cells; HepG2 cells; HeLa cells; HT-1080 cells; HCT-116 cells; Hu-h7 cells; Huvec cells; Molt 4 cells.

Primary immune cells can be obtained from a number of non-limiting sources, including peripheral blood mononuclear cells (PBMC), bone marrow, lymph node tissue, cord blood, thymus tissue, tissue from a site of infection, ascites, pleural effusion, spleen tissue, and from tumors, such as tumor infiltrating lymphocytes. In some embodiments, said immune cell can be derived from a healthy donor, from a patient diagnosed with cancer or from a patient diagnosed with an infection. In another embodiment, said cell is part of a mixed population of immune cells which present different phenotypic characteristics, such as comprising CD4, CD8 and CD56 positive cells. Primary immune cells are provided from donors or patients through a variety of methods known in the art, as for instance by leukapheresis techniques as reviewed by Schwartz J. et al. (Guidelines on the use of therapeutic apheresis in clinical practice-evidence-based approach from the Writing Committee of the American Society for Apheresis: the sixth special issue (2013) J Clin Apher. 28(3):145-284).

The immune cells derived from stem cells are also regarded as primary immune cells according to the present invention, in particular those deriving from induced pluripotent stem cells (iPS) [Yamanaka, K. et al. (2008). “Generation of Mouse Induced Pluripotent Stem Cells Without Viral Vectors”. Science. 322(5903):949-53]. Lentiviral expression of reprogramming factors has been used to induce multipotent cells from human peripheral blood cells [Staerk, J. et al. (2010). “Reprogramming of human peripheral blood cells to induced pluripotent stem cells”. Cell stem cell. 7(1):20-4] [Loh, Y H. et al. (2010). “Reprogramming of T cells from human peripheral blood”. Cell stem cell. 7(1):15-9].

According to a preferred embodiment of the invention, the immune cells are derived from human embryonic stem cells by techniques well known in the art that do not involve the destruction of human embryos [Chung et al. (2008) Human Embryonic Stem Cell lines generated without embryo destruction, Cell Stem Cell 2(2):113-117].

By “Genetic engineering” is meant any methods aiming to introduce, modify and/or withdraw genetic material from a cell. By “gene editing” is meant a genetic engineering allowing genetic material to be added, removed, or altered at specific locations (loci) in the genome, including punctual mutations. Gene editing generally involves sequence specific reagents.

By “sequence-specific reagent” is meant any active molecule that has the ability to specifically recognize a selected polynucleotide sequence at a genomic locus, referred to as “target sequence”, which is generally of at least 9 bp, more preferably of at least 10 bp and even more preferably of at least 12 pb in length, in view of modifying the expression of said genomic locus. Said expression can be modified by mutation, deletion or insertion into coding or regulatory polynucleotide sequences, by epigenetic change, such as by methylation or histone modification, or by interfering at the transcriptional level by interacting with transcription factors or polymerases.

Examples of sequence-specific reagents are endonucleases, RNA guides, RNAi, methylases, exonucleases, histone deacetylases, endonucleases, end-processing enzymes such as exonucleases, deaminases and more particularly cytidine deaminases such as those coupled with the CRISPR/cas9 or TALE systems to perform base editing (i.e. nucleotide substitution) without necessarily resorting to cleavage by nucleases as described for instance by Hess, G. T. et al. [Methods and applications of CRISPR-mediated base editing in eukaryotic genomes (2017) Mol Cell. 68(1): 26-43] or by Mok et al. [A bacterial cytidine deaminase toxin enables CRISPR-free mitochondrial base editing (2020) Nature 583:631-637].

According to a preferred aspect of the invention, said sequence-specific reagent is a sequence-specific nuclease reagent, such as a RNA guide coupled with a guided endonuclease.

The present invention aims to improve the therapeutic potential of immune cells through gene editing techniques, especially by gene targeted integration.

By “gene targeting integration” is meant any known site-specific methods allowing to insert, replace or correct a genomic coding sequence into a living cell.

According to a preferred aspect of the present invention, said gene targeted integration involves homologous gene recombination at the locus of the targeted gene to result the insertion or replacement of at least one exogenous nucleotide, preferably a sequence of several nucleotides (i.e. polynucleotide), and more preferably a coding sequence.
By “DNA target”, “DNA target sequence”, “target DNA sequence”, “nucleic acid target sequence”, “target sequence”, or “processing site” is intended a polynucleotide sequence that can be targeted and processed by a sequence-specific nuclease reagent according to the present invention. These terms refer to a specific DNA location, preferably a genomic location in a cell, but also a portion of genetic material that can exist independently to the main body of genetic material such as plasmids, episomes, virus, transposons or in organelles such as mitochondria as non-limiting example. As non-limiting examples of RNA guided target sequences, are those genome sequences that can hybridize the guide RNA which directs the RNA guided endonuclease to a desired locus.

“Rare-cutting endonucleases” are sequence-specific endonuclease reagents of choice, insofar as their recognition sequences generally range from 10 to 50 successive base pairs, preferably from 12 to 30 bp, and more preferably from 14 to 20 bp.

According to a preferred aspect of the invention, said endonuclease reagent is a nucleic acid encoding an “engineered” or “programmable” rare-cutting endonuclease, such as a homing endonuclease as described for instance by Arnould S., et al. [WO2004067736], a zinc finger nuclease (ZFN) as described, for instance, by Urnov F., et al. [Highly efficient endogenous human gene correction using designed zinc-finger nucleases (2005) Nature 435:646-651], a TALE-Nuclease as described, for instance, by Mussolino et al. [A novel TALE nuclease scaffold enables high genome editing activity in combination with low toxicity (2011) Nucl. Acids Res. 39(21):9283-9293], or a MegaTAL nuclease as described, for instance by Boissel et al. [MegaTALs: a rare-cleaving nuclease architecture for therapeutic genome engineering (2013) Nucleic Acids Research 42(4):2591-2601].

According to another embodiment, the endonuclease reagent is a RNA-guide to be used in conjunction with a RNA guided endonuclease, such as Cas9 or Cpf1, as per, inter alia, the teaching by Doudna, J., and Chapentier, E., [The new frontier of genome engineering with CRISPR-Cas9 (2014) Science 346 (6213):1077], which is incorporated herein by reference.

According to a preferred aspect of the invention, the endonuclease reagent is transiently expressed into the cells, meaning that said reagent is not supposed to integrate into the genome or persist over a long period of time, such as be the case of RNA, more particularly mRNA, proteins or complexes mixing proteins and nucleic acids (e.g.: Ribonucleoproteins).

An endonuclease under mRNA form is preferably synthetized with a cap to enhance its stability according to techniques well known in the art, as described, for instance, by Kore A. L., et al. [Locked nucleic acid (LNA)-modified dinucleotide mRNA cap analogue: synthesis, enzymatic incorporation, and utilization (2009) J Am Chem Soc. 131(18):6364-5].

In general, electroporation steps that are used to transfect primary immune cells, such as PBMCs are typically performed in closed chambers comprising parallel plate electrodes producing a pulse electric field between said parallel plate electrodes greater than 100 volts/cm and less than 5,000 volts/cm, substantially uniform throughout the treatment volume such as described in WO2004083379, which is incorporated by reference, especially from page 23, line 25 to page 29, line 11. One such electroporation chamber preferably has a geometric factor (cm⁻¹) defined by the quotient of the electrode gap squared (cm2) divided by the chamber volume (cm³), wherein the geometric factor is less than or equal to 0.1 cm⁻¹, wherein the suspension of the cells and the sequence-specific reagent is in a medium which is adjusted such that the medium has conductivity in a range spanning 0.01 to 1.0 milliSiemens. In general, the suspension of cells undergoes one or more pulsed electric fields. With the method, the treatment volume of the suspension is scalable, and the time of treatment of the cells in the chamber is substantially uniform.

Due to their higher specificity, TALE-nuclease or TALE-base editors have proven to be particularly appropriate sequence specific nuclease reagents for therapeutic applications, especially under heterodimeric forms—i.e. working by pairs with a “right” monomer (also referred to as “5′” or “forward”) and “left” monomer (also referred to as “3″” or “reverse”) as reported for instance by Mussolino et al. [TALEN facilitate targeted genome editing in human cells with high specificity and low cytotoxicity (2014) Nucl. Acids Res. 42(10): 6762-6773].

As previously stated, the sequence specific reagent is preferably under the form of nucleic acids, such as under DNA or RNA form encoding a rare cutting endonuclease a subunit thereof, but they can also be part of conjugates involving polynucleotide(s) and polypeptide(s) such as so-called “ribonucleoproteins”. Such conjugates can be formed with reagents as Cas9 or Cpf1 (RNA-guided endonucleases) as respectively described by Zetsche, B. et al. [Cpf1 Is a Single RNA-Guided Endonuclease of a Class 2 CRISPR-Cas System (2015) Cell 163(3): 759-771] and by Gao F. et al. [DNA-guided genome editing using the Natronobacterium gregoryi Argonaute (2016) Nature Biotech], which involve RNA or DNA guides that can be complexed with their respective nucleases.

“Exogenous sequence” refers to any nucleotide or nucleic acid sequence that was not initially present at the selected locus. This sequence may be homologous to, or a copy of, a genomic sequence, or be a foreign sequence introduced into the cell. By opposition “endogenous sequence” means a cell genomic sequence initially present at a locus. The exogenous sequence preferably codes for a polypeptide which expression confers a therapeutic advantage over sister cells that have not integrated this exogenous sequence at the locus. An endogenous sequence that is gene edited by the insertion of a nucleotide or polynucleotide as per the method of the present invention, in order to express a different polypeptide is broadly referred to as an exogenous coding sequence.

By using the above reagents and techniques, also detailed in the experimental section, the present invention concurs to develop methods for producing therapeutic cells by proceeding with one or several of the following steps:
Providing immune cells, preferably primary cells, from a donor or a patient;
Expressing into such cells an anti-MUC1 CAR as previously described, generally by introducing the anti-MUC1 CAR coding sequence into the cell's genome via a viral vector;
Introducing into such immune cells a sequence specific-reagent, such as rare-cutting endonuclease or a base editor to induce a modification (mutations or coding sequence insertion) at an endogenous gene locus; and/or
Introducing into said cell an exogenous coding sequence to improve the therapeutic potency thereof, in particular its immune properties.

According to some aspects of the invention the immune cells originate from a patient or a compatible donor, in which the anti-MUC1 CAR is expressed in view of performing so called “autologous” infusion of the engineered CAR positive immune cells. They can also be derived from stem cells, such as iPS cells, originating from such patient or compatible donor or from tumor infiltrating lymphocytes (TILL).

According to some aspects of the invention, the method aims to provide “off the shelf” compositions of immune cells, said immune cells being engineered for allogeneic therapeutic treatments.

By “allogeneic” is meant that the cells originate from a donor. They can be directly collected by apheresis or can produced/differentiated from stem cells. The fact that are used in allogeneic settings means that the engineered cells are infused into patients having a different haplotype.

Such immune cells are generally engineered to be less alloreactive and/or become more persistent with respect to their patient host. More specifically the present methods comprise the steps of reducing or inactivating TCR expression into T-cells, or stem cells to be derived into T-cells. This can be obtained by different sequence specific-reagents, such as by gene silencing or gene editing techniques (nuclease, base editing, RNAi . . . ).

The applicant has formerly made available robust protocols and gene editing strategies to produce allogeneic therapeutic grade T-cells from PBMCs, especially by providing very safe and specific endonuclease reagents under the form of TALE-nucleases (TALEN©). The production of so-called “universal T-cells”, which are [TCR]^{neg} T-cells from donors was achieved and successfully injected to patients with reduced Graft versus Host Disease (GVhD) [Poirot et al. (2015) Multiplex Genome-Edited T-cell Manufacturing Platform for “Off-the-Shelf” Adoptive T-cell Immunotherapies. Cancer. Res. 75 (18): 3853-3864] [Qasim, W. et al. (2017) Molecular remission of infant B-ALL after infusion of universal TALEN gene-edited CAR T cells. Science Translational 9(374)].

In preferred embodiments, the present invention provides with a method to engineer an immune cell, wherein at least one gene encoding TCRalpha or TCRbeta is inactivated in said immune cell, preferably by expression of a rare-cutting endonuclease, whereas an exogenous polynucleotide encoding anti-MUC1 CAR, such as previously described, is introduced into the genome of said cell for stable expression. Said exogenous sequence can be integrated at said locus encoding TCRalpha or TCRbeta, more preferably under transcriptional control of an endogenous promoter of TCRalpha or TCRbeta.

In further embodiments, the engineered immune cell can be further modified to confer resistance to at least one immune suppressive drug, such as by inactivating CD52 the target of anti-CD52 antibody (e.g.:alemtuzumab), which has been previously described with respect to the treatment of blood cancers for instance in WO2013176915.

While the use of anti-CD52 lymphodepletion agents had been limited so far to liquid tumor cancers [Quasim W. et al. (2019) Allogeneic CAR T cell therapies for leukemia Am J Hematol. 94:S50-S54.], one major aspect of the present invention is the use of genetically engineered lymphocytes made resistant to lymphodepletion regimen for the treatment of solid tumors.

Also the present invention provides engineered lymphocytes endowed with chimeric antigen receptors directed against solid tumors, especially against MUC1 positive cells, for their use in solid tumor cancer treatments in combination with, or preceded by, a lymphodepletion treatment step.

Such lymphodepletion regimen can comprise anti-CD52 reagents, such as Alemtuzumab, or purine analogues, as those used for treating blood cancers.

In preferred embodiments, the engineered lymphocytes endowed with anti-MUC1CAR described herein are made resistant to such lymphodepleting regimen by inhibiting or disrupting the expression of the molecules that are targeted by the lymphodepletion reagents, like for instance the antigen CD52 in the case of Alemtuzumab.

In further embodiments, the engineered immune cell can be further modified to confer resistance to and/or a chemotherapy drug, in particular a purine analogue drug, for example by inactivating DCK as described in WO201575195.

As indicated before, it is an important aspect of the present invention to treat solid tumor cancers with genetically engineered lymphocytes endowed with chimeric antigen receptors, which are made resistant to chemotherapy or lymphodepletion regimen. Such regimen can comprise antibodies targeting antigens present at the surface of immune cells, such as CD52, CD3, CD4, CD8, CD45, or other specific markers, but also less specific drugs such as purine analogues (ex: fludarabine and/or chlorofarabine) and glucocorticoids. One aspect of the invention is to make the engineered lymphocytes resistant to such regimen by inactivating or reducing the expression of the genes that encode at least one molecular target of these lymphodepletion reagents, for instance the gene DCK that metabolizes purine analogues or the genes encoding glucocorticoid receptors (GR).

The present invention is therefore more particularly focused on CAR positive cells, which expression of TCR, CD52 and/or DCK and/or GR is reduced, inactivated or deficient to make them less alloreactive and resistant to lymphodepletion regimen, for their allogeneic use in solid cancer treatments.

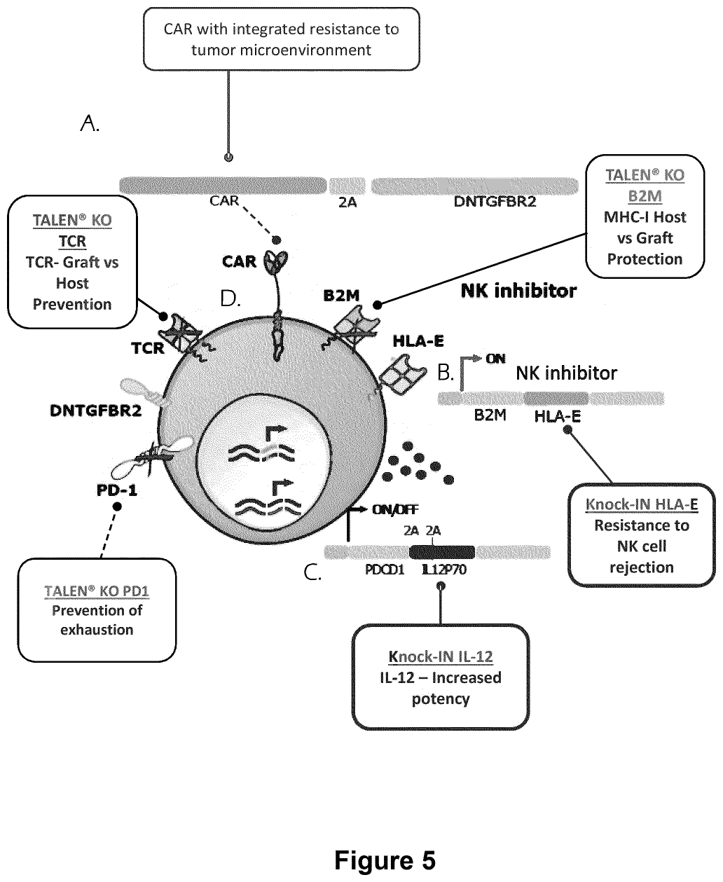

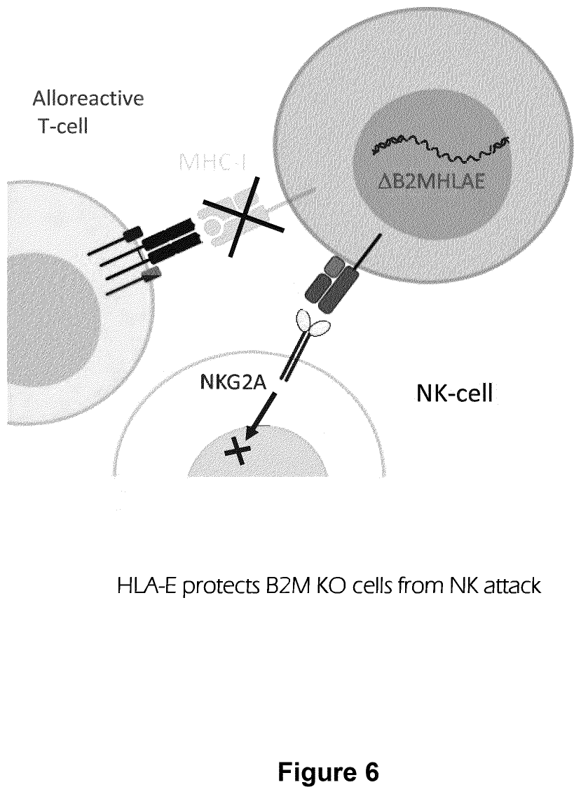

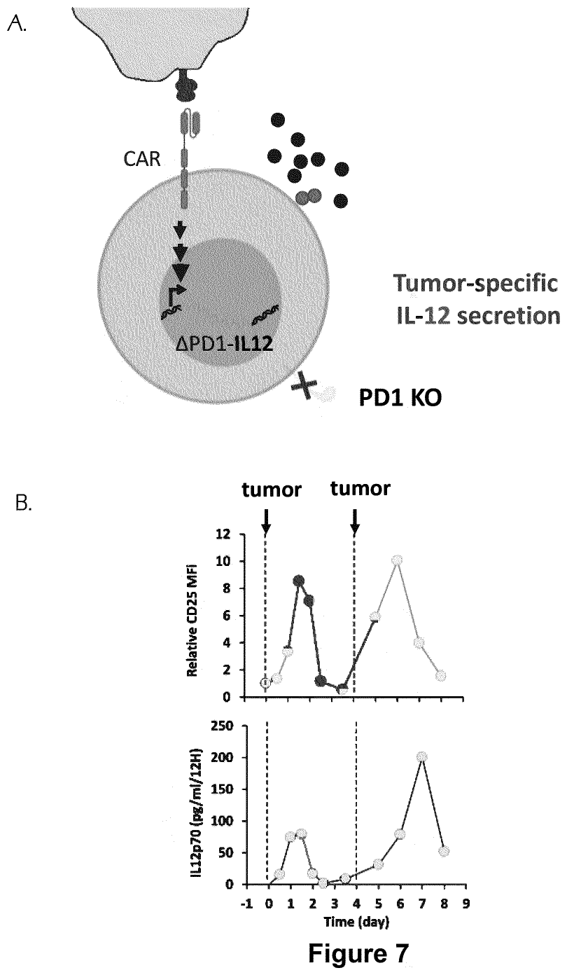

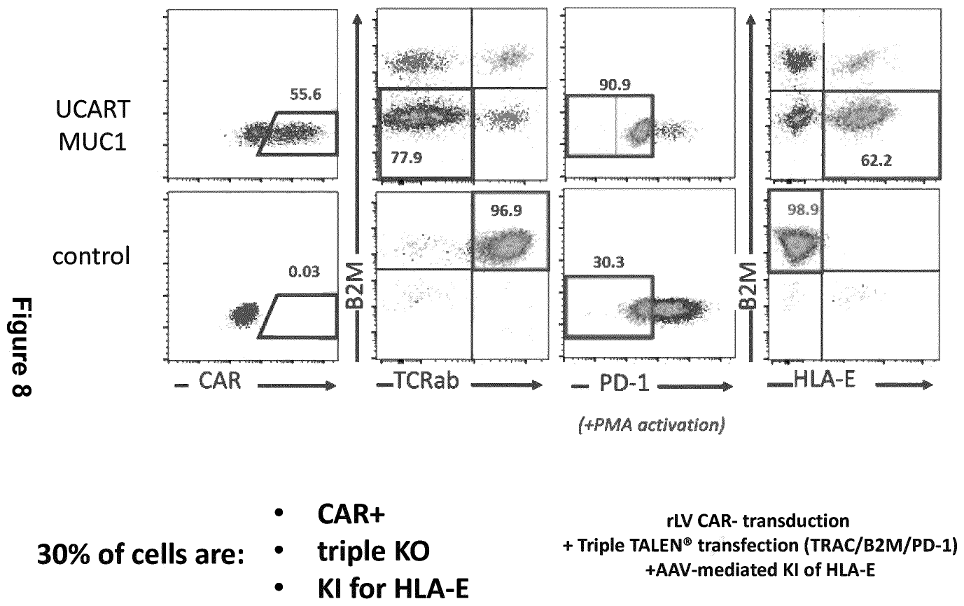

In order to increase potency of anti-MUC1 CAR-T cells and overcome solid tumors resistance mechanisms, the inventors have combined several genetic modifications—referred to herein as genetic attributes—to produce specifically dedicated to immunotherapy of MUC1 positive tumors. These genetic modifications, illustrated in FIG. 5, are more particularly selected from the following ones:

### 1) Limiting CAR-T Cells Exhaustion by Genetic Repression of Inactivation of Immune Checkpoint Genes, Such as PD1.

Tumor cells are known to upregulate expression of PDL1, a ligand which interacts with PD-1 on T-cells. This interaction effectively blocks T-cell mediated immune response preventing tumor clearance. Successful immune checkpoint therapies rely on blocking the interaction of PD1 with PDL-1, which allows the immune system to kill cancer cells. The invention thus prescribes to knockout PD1 in CAR-T cells to prevent PD-1/PDL-1 mediated immunosuppression. This modification in tandem with other attributes such as inducible IL-12 release as suggested below can alleviate issues of CAR-T cell exhaustion within tumor microenvironment.

2) Inducing Expression of IL-12 (or/and IL-2, IL-15 and/or IL-18).

IL-12 is a cytokine produced by variety of immune cells including dendritic cells, monocytes and macrophages (and some B-cells) that positively regulates Th1 responses and promotes survival and expansion of T-cells. The delivery of IL-12 by the CAR-T cells is particularly important for the potency of anti-tMUC1 CAR-T cells in allogeneic settings.

Since IL-12 is toxic when delivered at the systemic level, its encoding sequence can be integrated under an inducible IL-12 cassette that allows exclusive IL-12 release upon CAR-T cell activation in the presence of the tumor target. In this respect it is advantageous to express IL12 by using an exogenous sequence comprising IL-12a and IL-12b separated by a 2A self-cleaving peptide (IL-12a-P2A-IL-12b). The same could be applied with a sequence encoding IL-2, IL-15 or IL-18. As such, inducible cytokine release provides a safe way of boosting CAR-T potency as needed by the cells. According to preferred embodiments, the exogenous polynucleotide sequence expressing IL-2, IL-12, IL-15 and/or IL-18 can be delivered by and AAV vector and integrated into the PD1 locus as described in the examples, preferentially under transcriptional control of the endogenous PD1 promoter. Since PD-1 expression relies on CAR T-cell activation, IL-12 expression is thereby restricted to the tumor area where CAR T-cell function is enhanced.

As provided in the examples, PD-1 knockout together with IL-12 release significantly enhanced CAR-T cells function and intratumoral expansion resulting in anti-tumor activity against solid tumor which could not be treated with CAR-T cells without such modifications 3) Enhancing tumor infiltration by inducible expression of hyaluronidase enzymes.

Hyaluronan (HA) is a glycosaminoglycan that is an integral structural and signaling component of the extracellular matrix. Accumulation of HA is observed in variety of solid tumors including prostate, bladder, lung and breast cancer and is associated with poor clinical outcome. HA capsule increases hydrostatic pressure preventing T-cells infiltration and small drug diffusion in solid tumors, making treatment more challenging. While larger HA fragments enhance tumor progression for many cancer types, by promoting proliferation, preventing T-cell infiltration and by enhancing migration of tumor cells, small HA fragments (HA-oligos: 1-10kda) were shown to induce apoptosis of cancer cells. The inventors have successfully expressed hyaluronidase enzyme, capable of breaking down hyaluronan matrix in CAR-T cells in order to enhance CAR-T infiltration of solid tumors. Secretion of hyaluronidase enzymes by the CAR-T cells are also useful to induce bystander tumor cells killing through localized production of HA-oligos. Importantly, oligo-HA mediated apoptosis is not dependent on recognition of the CAR target antigen and thus has the potential to act on tumor cells lacking or expressing low level of antigen on cell surface.

Combination of CAR with hyaluronidase activity is analogous to antibody drug conjugate where bystander cancer cells lacking necessary antigen level can be killed as well due to exposure to oligo-HA. This strategy is thought to minimizes tumor escape due to antigen insufficiency or tumor heterogeneity.

The present invention provides engineered immune cells, preferably but not only anti-MUC1 CAR-T-cells, in which an exogenous sequence encoding hyaluronidase enzyme has been introduced. This hyaluronidase enzyme is preferably HYAL1 (Uniprot Q12794), HYAL2 (Q12891) and/or HYAL3 (SPAM1—Uniprot P38567) or similar functional hyaluronidase having at least 80% identity with the same. HYAL1, HYAL2 and SPAM1 sequences can also be modified with secretory tags can be implemented to enhance secretion into the tumor microenvironment.

According to some preferred embodiments, the immune cells can be are advantageously engineered to have the hyaluronidase enzyme secreted upon immune cell activation by integrating the hyaluronidase coding sequence at a locus selected from PD1, CD69, CD25 or GMCSF or any such loci which expression is induced upon engagement of the CAR with the targeted antigen.

The present invention also provides with AAV, preferably AAV6 vectors for mediated insertion of HYAL1, HYAL2 or SPAM1 into the immune cells, comprising an exogenous sequence encoding a functional hyaluronidase.

According to further embodiments, additional glycosidases human β-glucuronidase (GUSB) and β-N-acetylglucosaminidase (ENGASE) can be co-expressed to further shorten HA chains broken by the hyaluronidase enzyme.

The resulting CAR immune cells expressing hyaluronidases show better infiltration into solid tumors and can operate in synergy with CAR-T mediated killing by directly inducing tumor cell death through oligo-HA mediated apoptosis while sparing healthy tissues.

### 4) Mitigating Hypoxic and Low Nutrient Environment.

Tumor microenvironment not only is hypoxic but it also lacks nutrients, two features that hinder expansion of CAR-T cells. Recent work described a mechanism by which pro-inflammatory Th17 cells effectively expand in hypoxic and nutrient deficient environments causing inflammation [Wu, L. et al. (2020) Niche-Selective Inhibition of Pathogenic Th17 Cells by Targeting Metabolic Redundancy. Cell 182(3):641-654]. Th17 lymphocytes upregulate glucose phosphate isomerase 1 (GP11) to overcome hypoxic and low nutrient environments gaining a proliferative advantage. Whereas knockout of GP11, a metabolically redundant enzyme, had no effect on homeostatic Th17 cells that reside in nutrient sufficient and normoxic conditions, it severely compromised the proliferative ability of inflammatory Th17 cells in hypoxic and nutrient deficient environments. During anaerobic glycolysis lactate is generated. Until recently lactate has been considered as a waste product. However new findings demonstrate it can also be metabolized for energy with the help of phosphoenolpyruvate carboxykinase 1 (PCK1) which increases glycolytic flux, most likely through gluconeogenesis, resulting in consumption of lactose and increase in intracellular glucose [Yuan, Y. et al. (2020) PCK1 Deficiency Shortens the Replicative Lifespan of Saccharomyces cerevisiae through Upregulation of PFK1. Biomed Res. Int. Article ID 3858465]. Furthermore, PCK1 overexpression has been shown to facilitate formation and maintenance of CD8+Tm cells and overall to increase T cell competitiveness in acquiring nutrients [Ho, P. C. et al. (2015) Phosphoenolpyruvate Is a Metabolic Checkpoint of Anti-tumor T Cell Responses. Cell. 162(6):1217-1228].

The present invention provides engineered immune cells, preferably but not only anti-MUC1 CAR T-cells, in which an exogenous sequence encoding GP11 (Uniprot Q9BRB3), PCK1 (Uniprot P35558) and/or LDHA (Uniprot P00338) enzyme has been introduced or encoding any functional enzyme having at least 80% identity with the same. The inventors have expressed GP11 and PCK1 to boost immune cells in hypoxic and nutrient deficient environment of solid tumors. Genes encoding GP11 and PCK1 enzyme, or any functional similar enzyme, are preferably delivered using an AAV vector, especially AAV6 for its targeted insertion at the TRAC locus. Alternatively, the exogenous sequences can be delivered under the control of strong promoters such as EF1 alpha by using a lentiviral vector, as it is preferable to obtain a constitutive expression thereof. The expression of the two genes, can be achieved through the delivery of bicistronic construct carrying both genes separated by self-cleaving 2A peptide.

The above-mentioned modifications to boost CAR-T effect in solid tumors can be implemented in combination with standard engineering of allogeneic CAR-T, which more particularly include the following modifications:
TRAC knockout using gene editing for preventing graft vs. host disease;
B2M knockout using gene editing or silencing methods for preventing host vs. graft activity; and/or
HLA-E or HLA-G overexpression through gene targeted integration, such as by using AAV6, or by rLV delivery in order to prevent NK-cells from attacking B2M negative cells.

Together, complex engineering implementing several of the strategies mentioned above has the innovative potential to generate the next-generation of CAR-T cells capable of treating solid tumors.

Also the present invention provides with a method for manufacturing a population of engineered therapeutic immune cells for the treatment of solid tumors, comprising the steps of:
a) Providing immune cells originating from a patient or preferably from a donor;
b) Expressing in said cells an anti-MUC1 chimeric antigen receptor (CAR);
c) Introducing at least one genetic modification(s) in the genome of said cell, said modification(s) being selected from those leading to:
   Reduced or inactivated TCR expression;
   Reduced or inactivated B2M;
   Reduced interaction between TGFβ and TGFβR2;
   Reduced interaction between PD1 and PDL1; and/or
   Enhanced IL-12, IL-15 or IL-18 expression;
d) Expanding said cells to form a population of therapeutically effective population of immune cells.

The genetic modifications in this method is generally obtained by using sequence specific gene editing, such as rare-cutting endonucleases/nickases or base editors. The method can also comprise further genetically engineering the cells to enhance secretion of hyalurinase, such as HYAL1, HYAL2 and/or HYAL3 (SPAM1) or to enhance expression of other enzymes such as GP11, PCK1 and/or LDHA.

TABLE 9
Genetic attributes polypeptide sequences

| CAR Designation | Polypeptide Sequence |
|---|---|
| dnTGFβR2 | VLDKRRGRDPEMGGKPRRKNPQEGLYNELQKDKMAEAYSEIGMKGER |
| (SEQ ID NO: 59) | RRGKGHDGLYQGLSTATKDTYDALHMQALPPRGSGEGRGSLLTCGDVE |
| | ENPGPMGRGLLRGLWPLHIVLWTRIASTIPPHVQKSVNNDMIVTDNNGAV |
| | KFPQLCKFCDVRFSTCDNQKSCMSNCSITSICEKPQEVCVAVWRKNDEN |
| | ITLETVCHDPKLPYHDFILEDAASPKCIMKEKKKPGETFFMCSCSSDECN |
| | DNIIFSEEYNTSNPDLLLVIFQVTGISLLPPLGVAISVIIIFYCYRVNRQ |
| | QKLSS |
| HLA-E | HSLKYFHTSVSRPGRGEPRFISVGYVDDTQFVRFDNDAASPRMVPRAP |
| (SEQ ID NO: 60) | WMEQEGSEYWDRETRSARDTAQIFRVNLRTLRGYYNQSEAGSHTLQW |
| | MHGCELGPDRRFLRGYEQFAYDGKDYLTLNEDLRSWTAVDTAAQISEQ |
| | KSNDASEAEHQRAYLEDTCVEWLHKYLEKGKETLLHLEPPKTHVTHHPIS |
| | DHEATLRCWALGFYPAEITLTWQQDGEGHTQDTELVETRPAGDGTFQK |
| | WAAVVVPSGEEQRYTCHVQHEGLPEPVTLRWKPASQPTIPIVGIIAGLVL |
| | LGSVVSGAVVAAVIWRKKSSGGKGGSYYKAEWSDSAQGSESHSL |
| HLA-G | MWPPGSASQPPPSPAAATGLHPAARPVSLQCRLSMCPARSLLLVATLVL |
| (SEQ ID NO: 61) | LDHLSLARNLPVATPDPGMFPCLHHSQNLLRAVSNMLQKARQTLEFYPC |
| | TSEEIDHEDITKDKTSTVEACLPLELTKNESCLNSRETSFITNGSCLASRKT |
| | SFMMALCLSSIYEDLKMYQVEFKTMNAKLLMDPKRQIFLDQNMLAVIDEL |
| | MQALNFNSETVPQKSSLEEPDFYKTKIKLCILLHAFRIRAVTIDRVMSYLNA |
| | SGSGATNFSLLKQAGDVEENPGPMCHQQLVISWFSLVFLASPLVAIWEL |
| | KKDVYVVELDWYPDAPGEMVVLTCDTPEEDGITWTLDQSSEVLGSGKTL |
| | TIQVKEFGDAGQYTCHKGGEVLSHSLLLLHKKEDGIWSTDILKDQKEPKN |
| | KTFLRCEAKNYSGRFTCWWLTTISTDLTFSVKSSRGSSDPQGVTCGAAT |
| | LSAERVRGDNKEYEYSVECQEDSACPAAEESLPIEVMVDAVHKLKYENY |
| | TSSFFIRDIIKPDPPKNLQLKPLKNSRQVEVSWEYPDTWSTPHSYFSLTFC |
| | VQVQGKSKREKKDRVFTDKTSATVICRKNASISVRAQDRYYSSSWSEWA |
| | SVPCS |
| IL-12 | MWPPGSASQPPPSPAAATGLHPAARPVSLQCRLSMCPARSLLLVATLVL |
| (IL-12a-P2A- | LDHLSLARNLPVATPDPGMFPCLHHSQNLLRAVSNMLQKARQTLEFYPC |
| IL-12b) | TSEEIDHEDITKDKTSTVEACLPLELTKNESCLNSRETSFITNGSCLASRKT |
| (SEQ ID NO: 62) | SFMMALCLSSIYEDLKMYQVEFKTMNAKLLMDPKRQIFLDQNMLAVIDEL |
| | MQALNFNSETVPQKSSLEEPDFYKTKIKLCILLHAFRIRAVTIDRVMSYLNA |
| | SGSGATNFSLLKQAGDVEENPGPMCHQQLVISWFSLVFLASPLVAIWEL |
| | KKDVYVVELDWYPDAPGEMVVLTCDTPEEDGITWTLDQSSEVLGSGKTL |
| | TIQVKEFGDAGQYTCHKGGEVLSHSLLLLHKKEDGIWSTDILKDQKEPKN |
| | KTFLRCEAKNYSGRFTCWWLTTISTDLTFSVKSSRGSSDPQGVTCGAAT |
| | LSAERVRGDNKEYEYSVECQEDSACPAAEESLPIEVMVDAVHKLKYENY |
| | TSSFFIRDIIKPDPPKNLQLKPLKNSRQVEVSWEYPDTWSTPHSYFSLTFC |
| | VQVQGKSKREKKDRVFTDKTSATVICRKNASISVRAQDRYYSSSWSEWA |
| | SVPCS |
| | (Bold: P2A sequence) |
| IL-12a | MWPPGSASQPPPSPAAATGLHPAARPVSLQCRLSMCPARSLLLVATLVL |
| (SEQ ID NO: 63) | LDHLSLARNLPVATPDPGMFPCLHHSQNLLRAVSNMLQKARQTLEFYPC |
| | TSEEIDHEDITKDKTSTVEACLPLELTKNESCLNSRETSFITNGSCLASRKT |
| | SFMMALCLSSIYEDLKMYQVEFKTMNAKLLMDPKRQIFLDQNMLAVIDEL |
| | MQALNFNSETVPQKSSLEEPDFYKTKIKLCILLHAFRIRAVTIDRVMSYLNA |
| | S |
| IL-12b | MCHQQLVISWFSLVFLASPLVAIWELKKDVYVVELDWYPDAPGEMVVLT |
| (SEQ ID NO: 64) | CDTPEEDGITWTLDQSSEVLGSGKTLTIQVKEFGDAGQYTCHKGGEVLS |
| | HSLLLLHKKEDGIWSTDILKDQKEPKNKTFLRCEAKNYSGRFTCWWLTTI |
| | STDLTFSVKSSRGSSDPQGVTCGAATLSAERVRGDNKEYEYSVECQED |
| | SACPAAEESLPIEVMVDAVHKLKYENYTSSFFIRDIIKPDPPKNLQLKPLKN |
| | SRQVEVSWEYPDTWSTPHSYFSLTFCVQVQGKSKREKKDRVFTDKTSA |
| | TVICRKNASISVRAQDRYYSSSWSEWASVPCS |
| IL-15 | GTGSATMDWTWILFLVAAATRVHSGIHVFILGCFSAGLPKTEANWVNVIS |
| (IL-15-P2A-- | DLKKIEDLIQSMHIDATLYTESDVHPSCKVTAMKCFLLELQVISLESGDASI |
| IL-15RA) | HDTVENLIILANNSLSSNGNVTESGCKECEELEEKNIKEFLQSFVHIVQMFI |
| (SEQ ID NO: 65) | NTSGSGATNFSLLKQAGDVEENPGPGTGSATMDWTWILFLVAAATRVH |
| | SITCPPPMSVEHADIWVKSYSLYSRERYICNSGFKRKAGTSSLTECVLNK |
| | ATNVAHWTTPSLKCIRDPALVHQRPAPPSTVTTAGVTPQPESLSPSGKE |
| | PAASSPSSNNTAATTAAIVPGSQLMPSKSPSTGTTEISSHESSHGTPSQT |
| | TAKNWELTASASHQPPGVYPQGHSDTT |
| | (Bold: P2A sequence) |
| | (underlined: IgEss secretion motif) |
| IL-15a | GIHVFILGCFSAGLPKTEANWVNVISDLKKIEDLIQSMHIDATLYTESDVHP |
| (without IgE SS) | SCKVTAMKCFLLELQVISLESGDASIHDTVENLIILANNSLSSNGNVTESG |
| (SEQ ID NO: 66) | CKECEELEEKNIKEFLQSFVHIVQMFINTS |
| IL-15RA | ITCPPPMSVEHADIWVKSYSLYSRERYICNSGFKRKAGTSSLTECVLNKA |
| (without IgE SS) | TNVAHWTTPSLKCIRDPALVHQRPAPPSTVTTAGVTPQPESLSPSGKEP |
| (SEQ ID NO: 67) | AASSPSSNNTAATTAAIVPGSQLMPSKSPSTGTTEISSHESSHGTPSQTT |
| | AKNWELTASASHQPPGVYPQGHSDTT |
| IL-18 | MAAEPVEDNCINFVAMKFIDNTLYFIAEDDENLESDYFGKLESKLSVIRNL |
| (SEQ ID NO: 68) | NDQVLFIDQGNRPLFEDMTDSDCRDNAPRTIFIISMYKDSQPRGMAVTIS |
| | VKCEKISTLSCENKIISFKEMNPPDNIKDTKSDIIFFQRSVPGHDNKMQFES |
| | SSYEGYFLACEKERDLFKLILKKEDELGDRSIMFTVQNED |
| HYAL1 | MAAHLLPICALFLTLLDMAQGFRGPLLPNRPFTTVWNANTQWCLERHGV |
| (SEQ ID NO: 69) | DVDVSVFDVVANPGQTFRGPDMTIFYSSQLGTYPYYTPTGEPVFGGLPQ |
| | NASLIAHLARTFQDILAAIPAPDFSGLAVIDWEAWRPRWAFNWDTKDIYR |
| | QRSRALVQAQHPDWPAPQVEAVAQDQFQGAARAWMAGTLQLGRALRP |
| | RGLWGFYGFPDCYNYDFLSPNYTGQCPSGIRAQNDQLGWLWGQSRAL |
| | YPSIYMPAVLEGTGKSQMYVQHRVAEAFRVAVAAGDPNLPVLPYVQIFY |
| | DTTNHFLPLESCQAIKEYMDTTLGPFILNVTSGALLCSQALCSGHGRCVR |
| | RTSHPKALLLLNPASFSIQLTPGGGPLSLRGALSLEDQAQMAVEFKCRCY |
| | PGWQAPWCERKSMW |
| HYAL2 | MRAGPGPTVTLALVLAVSWAMELKPTAPPIFTGRPFVVAWDVPTQDCGP |
| (SEQ ID NO: 70) | RLKVPLDLNAFDVQASPNEGFVNQNITIFYRDRLGLYPREDSAGRSVHGG |
| | VPQNVSLWAHRKMLQKRVEHYIRTQESAGLAVIDWEDWRPVWVRNWQ |
| | DKDVYRRLSRQLVASRHPDWPPDRIVKQAQYEFEFAAQQFMLETLRYVK |
| | AVRPRHLWGFYLFPDCYNHDYVQNWESYTGRCPDVEVARNDQLAWLW |
| | AESTALFPSVYLDETLASSRHGRNFVSFRVQEALRVARTHHANHALPVY |
| | VFTRPTYSRRLTGLSEMDLISTIGESAALGAAGVILWGDAGYTTSTETCQ |
| | YLKDYLTRLLVPYVVNVSWATQYCSRAQCHGHGRCVRRNPSASTFLHLS |
| | TNSFRLVPGHAPGEPQLRPVGELSWADIDHLQTHFRCQCYLGWSGEQC |
| | QWDHRQAAGGASEAWAGSHLTSLLALAALAFTWTL |
| HYAL3 (SPAM1) | MGVLKFKHIFFRSFVKSSGVSQIVFTFLLIPCCLTLNFRAPPVIPNVPFLWA |
| (SEQ ID NO: 71) | WNAPSEFCLGKFDEPLDMSLFSFIGSPRINATGQGVTIFYVDRLGYYPYID |
| | SITGVTVNGGIPQKISLQDHLDKAKKDITFYMPVDNLGMAVIDWEEWRPT |
| | WARNWKPKDVYKNRSIELVQQQNVQLSLTEATEKAKQEFEKAGKDFLVE |
| | TIKLGKLLRPNHLWGYYLFPDCYNHHYKKPGYNGSCFNVEIKRNDDLSW |
| | LWNESTALYPSIYLNTQQSPVAATLYVRNRVREAIRVSKIPDAKSPLPVFA |
| | YTRIVFTDQVLKFLSQDELVYTFGETVALGASGIVIWGTLSIMRSMKSCLL |
| | LDNYMETILNPYIINVTLAAKMCSQVLCQEQGVCIRKNWNSSDYLHLNPD |
| | NFAIQLEKGGKFTVRGKPTLEDLEQFSEKFYCSCYSTLSCKEKADVKDTD |
| | AVDVCIADGVCIDAFLKPPMETEEPQIFYNASPSTLSATMFIWRLEVWDQ |
| | GISRIGFF |
| GPI1 | MVALCSLQHLGSSDPRALPTLPTATSGQRPAKRRRKSPAMAALTRDPQF |
| (SEQ ID NO: 72) | QKLQQWYREHRSELNLRRLFDANKDRFNHFSLTLNTNHGHILVDYSKNL |
| | VTEDVMRMLVDLAKSRGVEAARERMFNGEKINYTEGRAVLHVALRNRSN |
| | TPILVDGKDVMPEVNKVLDKMKSFCQGPLMVTEALKPYSSGGPRVWYV |
| | SNIDGTHIAKTLAQLNPESSLFIIASKTFTTQETITNAETAKEWFLQAAKDP |
| | SAVAKHFVALSTNTTKVKEFGIDPQNMFEFWDWVGGRYSLWSAIGLSIAL |
| | HVGFDNFEQLLSGAHWMDQHFRTTPLEKNAPVLLALLGIWYINCFGCET |
| | HAMLPYDQYLHRFAAYFQQGDMESNGKYITKSGTRVDHQTGPIVWGEP |
| | GTNGQHAFYQLIHQGTKMIPCDFLIPVQTQHPIRKGLHHKILLANFLAQTE |
| | ALMRGKSTEEARKELQAAGKSPEDLERLLPHKVFEGNRPTNSIVFTKLTP |
| | FMLGALVAMYEHKIFVQGIIWDINSFDQWGVELGKQLAKKIEPELDGSAQ |
| | VTSHDASTNGLINFIKQQREARVQ |
| PCK1 | MPPQLQNGLNLSAKVVQGSLDSLPQAVREFLENNAELCQPDHIHICDGS |
| (SEQ ID NO: 73) | EEENGRLLGQMEEEGILRRLKKYDNCWLALTDPRDVARIESKTVIVTQEQ |
| | RDTVPIPKTGLSQLGRWMSEEDFEKAFNARFPGCMKGRTMYVIPFSMG |
| | PLGSPLSKIGIELTDSPYVVASMRIMTRMGTPVLEAVGDGEFVKCLHSVG |
| | CPLPLQKPLVNNWPCNPELTLIAHLPDRREIISFGSGYGGNSLLGKKCFAL |
| | RMASRLAKEEGWLAEHMLILGITNPEGEKKYLAAAFPSACGKTNLAMMN |
| | PSLPGWKVECVGDDIAWMKFDAQGHLRAINPENGFFGVAPGTSVKTNP |
| | NAIKTIQKNTIFTNVAETSDGGVYWEGIDEPLASGVTITSWKNKEWSSED |
| | GEPCAHPNSRFCTPASQCPIIDAAWESPEGVPIEGIIFGGRRPAGVPLVY |
| | EALSWQHGVFVGAAMRSEATAAAEHKGKIIMHDPFAMRPFFGYNFGKYL |
| | AHWLSMAQHPAAKLPKIFHVNWFRKDKEGKFLWPGFGENSRVLEWMFN |
| | RIDGKASTKLTPIGYIPKEDALNLKGLGHINMMELFSISKEFWEKEVEDIEK |
| | YLEDQVNADLPCEIEREILALKQRISQM |
| LDHA | MATLKDQLIYNLLKEEQTPQNKITVVGVGAVGMACAISILMKDLADELALV |
| (SEQ ID NO: 74) | DVIEDKLKGEMMDLQHGSLFLRTPKIVSGKDYNVTANSKLVIITAGARQQE |
| | GESRLNLVQRNVNIFKFIIPNVVKYSPNCKLLIVSNPVDILTYVAWKISGFP |
| | KNRVIGSGCNLDSARFRYLMGERLGVHPLSCHGWVLGEHGDSSVPVWS |
| | GMNVAGVSLKTLHPDLGTDKDKEQWKEVHKQVVESAYEVIKLKGYTSW |
| | AIGLSVADLAESIMKNLRRVHPVSTMIKGLYGIKDDVFLSVPCILGQNGISD |
| | LVKVTLTSEEEARLKKSADTLWGIQKELQF |

In further embodiments, the engineered immune cell can be further modified to improve its persistence or its lifespan into the patient, in particular inactivating a gene encoding MHC-I component(s) such as HLA or β2m, such as described in WO2015136001 or by Liu, X. et al. [CRISPR-Cas9-mediated multiplex gene editing in CAR-T cells (2017) Cell Res 27:154-157].

According to a preferred aspect of the invention, the engineered immune cell is mutated to improve its CAR-dependent immune activation, in particular to reduce or suppress the expression of immune checkpoint proteins and/or their receptors thereof, such as PD1 or CTLA4, as described in WO2014184744.

### Combination of Expression of Anti-MUC1 CAR and Disruption of TGFbRII Signalling Pathway in the Therapeutic Engineered Immune Cells

The present invention more particularly combines the expression of an exogenous sequence encoding anti-MUC1 CAR as previously described, with another exogenous sequence encoding an inhibitor of a TGFbeta receptor, especially an inhibitor of TGFβRII.

TGFbeta receptors (Uniprot—P37173) have been described as having preponderant roles in tumor microenvironment [Papageorgis, P. et al. (2015). Role of TGFβ in regulation of the tumor microenvironment and drug delivery (Review). International Journal of Oncology, 46:933-943].

Although, the exact role of TGFbeta receptors in tumorogenesis remains controversial, the inventors have found that co-expressing anti-MUC1 chimeric antigen receptor (CAR) with another exogenous genetic sequence encoding an inhibitor of TGFBRII signalling and/or inactivating or reducing TGFbeta receptor signalling by using a sequence-specific reagent, was leading to an improved therapeutic potency of the engineered immune cells. In particular, the inventors have used two different approaches to impair TGFβRII signalling pathway, which may be combined together:
expression of an inactive ligand of TGFβRII, such as a dominant negative TGFβRII (SEQ ID NO:26), as described by Hiramatsu, K., et al. [Expression of dominant negative TGF-β receptors inhibits cartilage formation in conditional transgenic mice (2011) J. Bone. Miner. Metab. 29: 493]. or a similar inactive form of TGFβRII having at least 80%, preferably at least 90%, more preferably at least 95% identity with the polypeptide sequence SEQ ID NO:26.
and/or
inactivation of endogenous genetic sequence of TGFβRII, in particular by using a rare cutting endonuclease, such as a TALE-nuclease or RNA-guided endonuclease (e.g.: Cas9 or Cpf1).

An anti-TGFβRII IgG, monoclonal antibody that inhibits receptor-mediated signaling activation such as LY3022859 [Tolcher, A. W. et al. (2017) A phase 1 study of anti-TGFβ receptor type-II monoclonal antibody LY3022859 in patients with advanced solid tumors Cancer Chemother Pharmacol. 79(4):673-680] can also be used to inhibit TGFbeta receptor signalling in combination of the CAR as per the present invention.

The present application discloses herein a selection of TALE-nucleases, which are particularly specific to a selection of target sequences within the TGFβRII gene. These TALE-nucleases have displayed highest TGFβRII knock-out efficiency with very little off-target cleavage resulting into large populations of viable engineered cells, sufficient for dosing several patients. These preferred TALE-nuclease and their corresponding target sequences are listed in Table 10.

TABLE 10
TALE-nuclease target sequences for TGFβRII gene.

| TALEN | Target Sequence into TGFβRII gene |
|---|---|
| T003387 | TTTTGTTTCCCCATCAGAATATAACACCAGCAA |
| (SEQ ID NO: 86) | TCCTGACTTGTTGCTA |
| T003401 | TCCCTATGAGGAGTATGCCTCTTGGAAGACAGA |
| (SEQ ID NO: 87) | GAAGGACATCTTCTCA |
| T003400 | TGGCAGTCAAGATCTTTCCCTATGAGGAGTATG |
| (SEQ ID NO: 88) | CCTCTTGGAAGACAGA |
| T003405 | TGTGGGAGGCCCAAGATGCCCATCGTGCACAGG |
| (SEQ ID NO: 89) | GACCTCAAGAGCTCCA |

RNA guides avec also been designed to inactivate TGFβRII gene by using Cas9 nuclease reagent. Their corresponding respective target sequences are disclosed in Table 11.

TABLE 11
CRISPR target sequences for TGFβRII gene.

| | SEQ ID | PAM |
|---|---|---|
| sgRNA Sequence with PAM | NO: # | Sequence |
| ACAGTGATCACACTCCATGTGGG | 90 | GGG |
| GCAGAAGCTGAGTTCAACCTGGG | 91 | GGG |
| AGGTTAGGTCGTTCTTCACGAGG | 92 | AGG |
| AAAGCGACCTTTCCCCACCAGGG | 93 | GGG |
| TGGATGACCTGGCTAACAGTGGG | 94 | GGG |
| CCTGGGAAACCGGCAAGACGCGG | 95 | CGG |
| ACAGATATGGCAACTCCCAGTGG | 96 | TGG |
| GTGGAGGTGAGCAATCCCCCGGG | 97 | GGG |
| ACCTACAGGAGTACCTGACGCGG | 98 | CGG |
| GTGATCACACTCCATGTGGGAGG | 99 | AGG |
| GCTGGTGTTATATTCTGATGGGG | 100 | GGG |
| CACAGTGATCACACTCCATGTGG | 101 | TGG |
| CACATGGAGTGTGATCACTGTGG | 102 | TGG |
| CAGAGTAGGGTCCAGACGCAGGG | 103 | GGG |
| GCTTCTGCTGCCGGTTAACGCGG | 104 | CGG |
| GTGGATGACCTGGCTAACAGTGG | 105 | TGG |
| GGGAAAGCCCAAAGTCACACAGG | 106 | AGG |
| AATATGACTAGCAACAAGTCAGG | 107 | AGG |
| ATGGAGTGTGATCACTGTGGAGG | 108 | AGG |
| ATCACCGCCTTCCACGCCAAGGG | 109 | GGG |
| AGGAGCGGAAGACGGAGTTGGGG | 110 | GGG |
| CCACGCCAAGGGCAACCTACAGG | 111 | AGG |
| CAAGATGCCCATCGTGCACAGGG | 112 | GGG |
| GATATGGCAACTCCCAGTGGTGG | 113 | TGG |
| AGCAGAAGCTGAGTTCAACCTGG | 114 | TGG |
| CCTGTAGGTTGCCCTTGGCGTGG | 115 | TGG |
| GTGAGCAATCCCCCGGGCGAGGG | 116 | GGG |
| ACAGAGTAGGGTCCAGACGCAGG | 117 | AGG |
| GACGCGGCATGTCATCAGCTGGG | 118 | GGG |
| GGTCATCCACAGACAGAGTAGGG | 119 | GGG |
| AGTCAAGATCTTTCCCTATGAGG | 120 | AGG |
| GAACATACTCCAGTTCCTGACGG | 121 | CGG |
| ACGTGGAGCTGATGTCAGAGCGG | 122 | CGG |
| GGGGAAAGGTCGCTTTGCTGAGG | 123 | AGG |
| TCTGGACCCTACTCTGTCTGTGG | 124 | TGG |
| TGGGCAGCTCCCTCGCCCGGGGG | 125 | GGG |
| AGCTGATGACATGCCGCGTCAGG | 126 | AGG |
| GCCGCGTCAGGTACTCCTGTAGG | 127 | AGG |
| CAAGAGGCATACTCCTCATAGGG | 128 | GGG |
| GGTGAGCAATCCCCCGGGCGAGG | 129 | AGG |
| TTGCTGGTGTTATATTCTGATGG | 130 | TGG |
| CCTATGAGGAGTATGCCTCTTGG | 131 | TGG |
| TGCTGGTGTTATATTCTGATGGG | 132 | GGG |
| CGAGGATATTGGAGCTCTTGAGG | 133 | AGG |
| GTTGATGTTGTTGGCACACGTGG | 134 | TGG |
| TGACGCGGCATGTCATCAGCTGG | 135 | TGG |
| TTGAGCTGGACACCCTGGTGGGG | 136 | GGG |
| TTCAGAGCAGTTTGAGACAGTGG | 137 | TGG |
| GCGGCATGTCATCAGCTGGGAGG | 138 | AGG |
| AGGTCATCCACAGACAGAGTAGG | 139 | AGG |
| GAAGATGATGATGACAGATATGG | 140 | TGG |
| TGACCTGGCTAACAGTGGGCAGG | 141 | AGG |
| TCTACTGCTACCGCGTTAACCGG | 142 | CGG |
| GTCCTTCTCTGTCTTCCAAGAGG | 143 | AGG |
| ATTGAGCTGGACACCCTGGTGGG | 144 | GGG |
| GTCGTTCTTCACGAGGATATTGG | 145 | TGG |
| CATCAGCCTCCTGCCACCACTGG | 146 | TGG |
| AGTTCCTGACGGCTGAGGAGCGG | 147 | CGG |
| ACTCCAGTTCCTGACGGCTGAGG | 148 | AGG |
| CTTGAGGTCCCTGTGCACGATGG | 149 | TGG |
| TTGAGGTCCCTGTGCACGATGGG | 150 | GGG |
| CCGCGTCTTGCCGGTTTCCCAGG | 151 | AGG |
| GTTGCCCTTGGCGTGGAAGGCGG | 152 | CGG |
| CTTTGGGCTTTCCCTGCGTCTGG | 153 | TGG |
| GATCACCGCCTTCCACGCCAAGG | 154 | AGG |
| TGAAGTGTTCTGCTTCAGCTTGG | 155 | TGG |
| CTGTGCACGATGGGCATCTTGGG | 156 | GGG |
| GGCATCTTGGGCCTCCCACATGG | 157 | TGG |
| TTACCTGCCCACTGTTAGCCAGG | 158 | AGG |
| ATCAGCCTCCTGCCACCACTGGG | 159 | GGG |
| TCGCTTTGCTGAGGTCTATAAGG | 160 | AGG |
| AGTCACACAGGCAGCAGGTTAGG | 161 | AGG |
| GAGGAGCGGAAGACGGAGTTGGG | 162 | GGG |
| TGGGCAGCAGCTCTGTGTTGTGG | 163 | TGG |
| TAGCAACAAGTCAGGATTGCTGG | 164 | TGG |
| CCAAGATGCCCATCGTGCACAGG | 165 | AGG |
| TGTGGAGGTGAGCAATCCCCCGG | 166 | CGG |
| TGCCTCTTGGAAGACAGAGAAGG | 167 | AGG |
| GCCCATTGAGCTGGACACCCTGG | 168 | TGG |
| CTGAGTTCAACCTGGGAAACCGG | 169 | CGG |
| TGGGAGGACCTGCGCAAGCTGGG | 170 | GGG |
| GTACTCCTGTAGGTTGCCCTTGG | 171 | TGG |
| TCTTCCGCTCCTCAGCCGTCAGG | 172 | AGG |
| CTGGGCAGCTCCCTCGCCCGGGG | 173 | GGG |
| CATTGAGCTGGACACCCTGGTGG | 174 | TGG |
| TGGCAACTCCCAGTGGTGGCAGG | 175 | AGG |
| CAACTCCCAGTGGTGGCAGGAGG | 176 | AGG |
| CGAGCACTGTGCCATCATCCTGG | 177 | TGG |
| GCGGTCATCTTCCAGGATGATGG | 178 | TGG |
| AGAGCTGCTGCCCATTGAGCTGG | 179 | TGG |
| ACCAGGGTGTCCAGCTCAATGGG | 180 | GGG |
| CTGGACACCCTGGTGGGGAAAGG | 181 | AGG |
| CAAAGCGACCTTTCCCCACCAGG | 182 | AGG |
| GACGGCTGAGGAGCGGAAGACGG | 183 | CGG |
| TACTCTGTCTGTGGATGACCTGG | 184 | TGG |
| CGGCAAGACGCGGAAGCTCATGG | 185 | TGG |
| CCCAAAGTCACACAGGCAGCAGG | 186 | AGG |
| TAGGTTGCCCTTGGCGTGGAAGG | 187 | AGG |
| TGAGGAGCGGAAGACGGAGTTGG | 188 | TGG |
| CCTGTGCACGATGGGCATCTTGG | 189 | TGG |
| GGGAGCTGCCCAGCTTGCGCAGG | 190 | AGG |
| TTGAACTCAGCTTCTGCTGCCGG | 191 | CGG |
| CCAAGAGGCATACTCCTCATAGG | 192 | AGG |
| CATGAGCTTCCGCGTCTTGCCGG | 193 | CGG |
| CGGAGTTGGGGAAACAATACTGG | 194 | TGG |
| GCTCCTCAGCCGTCAGGAACTGG | 195 | TGG |
| CACCAGGGTGTCCAGCTCAATGG | 196 | TGG |
| CTAGTCATATTTCAAGTGACAGG | 197 | AGG |
| GCTGGGCAGCTCCCTCGCCCGGG | 198 | GGG |
| TGTCAGAGCGGTCATCTTCCAGG | 199 | AGG |
| CTGGGAGGACCTGCGCAAGCTGG | 200 | TGG |
| AGCTGGGCAGCTCCCTCGCCCGG | 201 | CGG |
| TGTGTTGTGGTTGATGTTGTTGG | 202 | TGG |
| CCTGCTGCCTGTGTGACTTTGGG | 203 | GGG |
| ACCTGCTGCCTGTGTGACTTTGG | 204 | TGG |

The present invention thus encompasses the use of a TALE-nuclease or RNA-guided endonuclease designed to bind any of the target sequences SEQ ID NO:90 to 204 referred to in table 10 or 11 for the inactivation or reducing of expression of TGFβRII for the production of therapeutic immune cells within the teaching of the present specification.

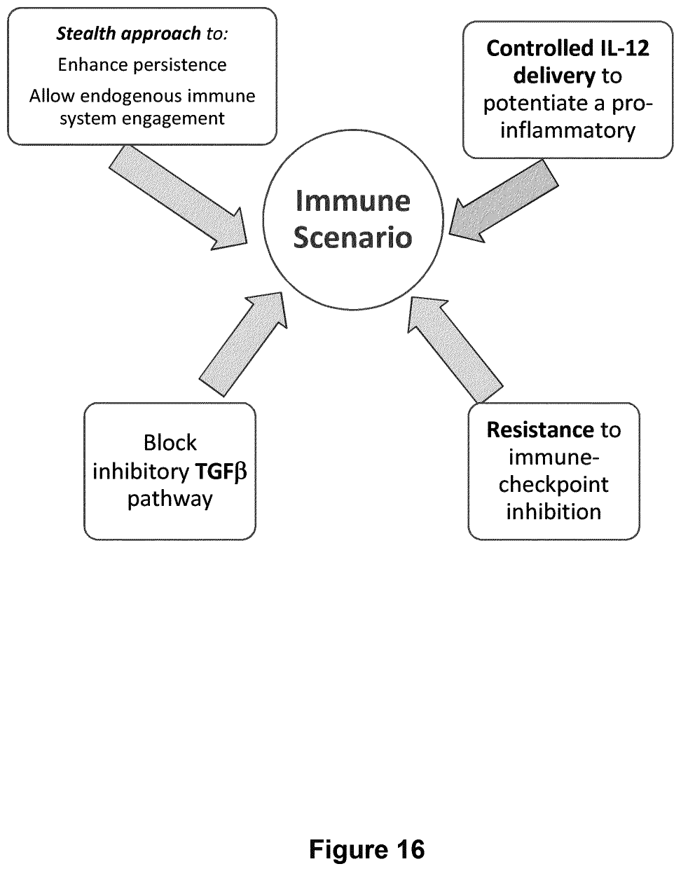

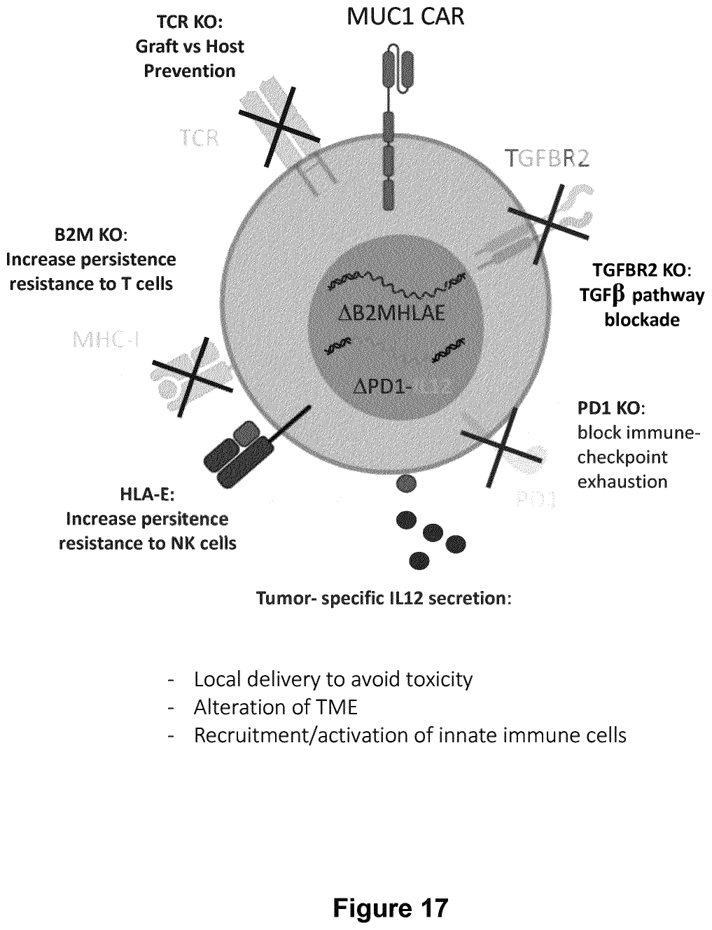

The present invention also pertains to engineered immune cells comprising an exogenous polynucleotide encoding a nuclease, such as one referred to before, to inactivate or reduce the expression of its endogenous TGFβRII gene. This strategy is more particularly illustrated in FIGS. 16 and 17.

As shown in the experimental section herein, the inactivation of TGFβRII by expression into the cells of sequence specific reagents, such as rare-cutting endonucleases or base editors leads to anti-MUC1 CAR T-cells with higher antitumoral activity.

Preferred specific reagents in this respect are TALE-Nucleases or TALE base editors to introduce mutations into one target sequence of TGFBRII selected from SEQ ID NO:86, 87, 88 or 89, more preferably SEQ ID NO:89, especially TALE nucleases having at least one monomer showing at least 95% identity with one of the polypeptide sequence SEQ ID NO: 223 and SEQ ID NO:224.

According to such embodiments, the invention provides with methods to produce CAR immune cells active against solid tumors, wherein at least one allele of each TCR (ex: TRAC), PD1, B2M and TGFBRII genes has been mutated by using a sequence specific reagent (nuclease or base editor) to reduce or inactivate their expression. Such methods preferably comprise two electroporation steps where the polynucleotide sequences encoding those sequence specific reagent, preferably under mRNA form, are introduced into the cells. These two electroporation steps are preferably spaced by an interval of at least 48 hours, more preferably of at least 72 hours and even more preferably of at least 96 hours. The inventors have obtained particular good results by introducing the specific reagents targeting TCR and B2M during the first electroporation step, and the specific reagents targeting PD1 and TGFBRII during the second electroporation step. This occurred to reduce cytotoxicity which permitted to produce higher yields of quadruple mutated engineered cells.

However, it may happen that several tumor resident cells produce TGF-β which has a potent immunosuppressive effect and can block both CAR-T proliferation and effector function. In order to prevent this blockade, the invention provides the delivery of dominant negative TGFBR2 (dnTGFBR2). Dominant negative TGFBR2 is a receptor lacking the intracellular signalling domain and is thus incapable of transducing TGF-B signal while retaining high affinity for TGF-β. dnTGFBR2 thus acts as a sponge to sequester TGF-β from CAR-T, also ameliorating immunosuppressive effect of TGF-β1. Preferred delivery of dnTGFBR2 has been successfully achieved through rLV, either as a single entity or as a bicistronic construct expressing CAR and separated by 2A self-cleaving peptide. However this could also be achieved via targeted integration, such as with AAV vectors in inducible loci (e.g. PD-1, CD25 . . . ) with the purpose of assist re-programming the immune-suppressive solid tumor microenvironment.

The present application thus reports engineered immune cells, especially CAR immune cells, into which an exogenous sequence encoding an inhibitor of TGFbeta receptor has been introduced, more particularly a sequence encoding a dominant negative TGFbeta receptor. Such cells are more particularly dedicated to the treatment of solid tumors, especially MUC1 positive tumors.

Accordingly, the present application also claims vectors, especially viral vectors, such as lentiviral vectors or AAV vectors as described in the art, comprising at least a polynucleotide sequence encoding a dominant negative TGFβRII, and optionally, an anti-MUC1 chimeric antigen receptor. In preferred embodiments, said vectors comprise a first polynucleotide sequence encoding said d152ominant negative TGFβRII, a second polynucleotide sequence encoding 2A self-cleaving peptide and a third one encoding said specific anti-MUC1 chimeric antigen receptor.

Targeted insertion into immune cells can be significantly improved by using AAV vectors, especially vectors from the AAV6 family or chimeric vectors AAV2/6 previously described by Sharma A., et al. [Transduction efficiency of AAV 2/6, 2/8 and 2/9 vectors for delivering genes in human corneal fibroblasts. (2010) Brain Research Bulletin. 81 (2-3): 273-278].

One aspect of the present invention is thus the transduction of AAV vectors comprising Anti-MUC1CAR coding sequence in human primary immune cells, in conjunction with the expression of sequence-specific endonuclease reagents, such as TALE endonucleases, to increase gene integration at the loci previously cited.

According to a preferred aspect of the invention, sequence specific endonuclease reagents can be introduced into the cells by transfection, more preferably by electroporation of mRNA encoding said sequence specific endonuclease reagents.

The obtained insertion of the exogenous nucleic acid sequence may result into the introduction of genetic material, correction or replacement of the endogenous sequence, more preferably “in frame” with respect to the endogenous gene sequences at that locus.

According to another aspect of the invention, from 10⁵ to 10⁷, preferably from 10⁶ to 10⁷, more preferably about 5·10⁶ viral genomes viral genomes are transduced per cell.

According to another aspect of the invention, the cells can be treated with proteasome inhibitors, such as Bortezomib or HDAC inhibitors to further help homologous recombination.

As one object of the present invention, the AAV vector used in the method can comprise an exogenous coding sequence that is promoter less, said coding sequence being any of those referred to in this specification.

The present invention also provides with an efficient method for obtaining primary immune cells, which can be gene edited in various gene loci more particularly involved into host-graft interaction and recognition. Other loci may also be edited in view of improving the activity, the survival or the life-time of the engineered primary cells, especially primary T cells.

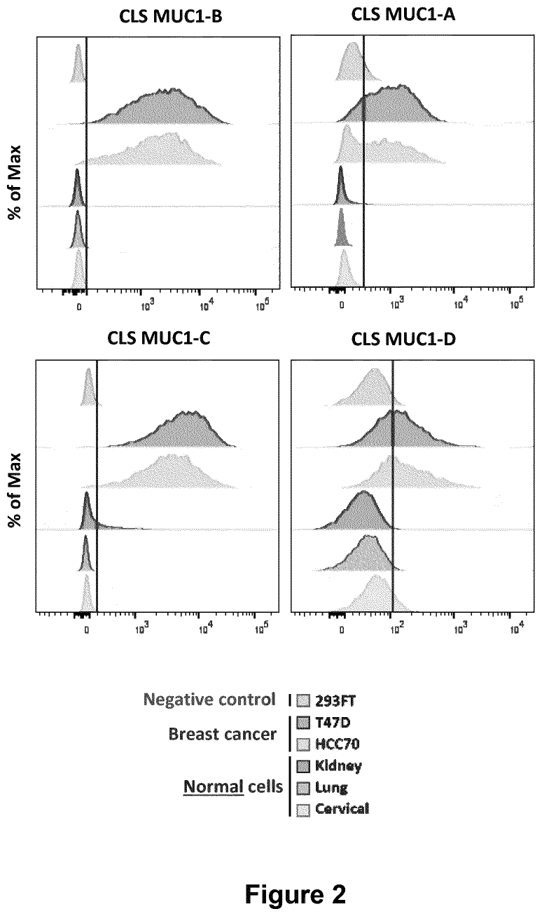

FIG. 2 maps the main cell functions that can be modified by gene editing according to the present invention to improve the efficiency of the engineered immune cells. Any gene inactivation listed under each function can be combined with another to obtain a synergistic effect on the overall therapeutic potency of the immune cells.

The present invention provides more particularly with combinations of genetic modifications (genotypes) into immune cells prompt to improve immune cells potency against solid tumor, especially against anti-MUC1 positive malignant cells, such as:
[anti-MUC1 CAR]⁺,
[anti-MUG1 CAR]⁺ [dnTGFβRII]⁺
[anti-MUC1 CAR]⁺ [dnTGFβRII]⁺ [TCR]⁻,
[anti-MUC1 CAR]⁺ [dnTGFβRII]⁺ [TGFβRII]⁻ [TCR]⁻,
[anti-MUC1 CAR]⁺ [TGFβRII]⁻,
[anti-MUC1 CAR]⁺ [TGFβRII]⁻ [TCR]⁻,
[anti-MUC1 CAR]⁺ [β2m]⁻,
[anti-MUC1 CAR]⁺ [dnTGFβRII]⁺ [β2m]⁻,
[anti-MUC1 CAR]⁺ [TGFβRII]⁻ [β2m]⁻,
[anti-MUC1 CAR]⁺ [dnTGFβRII]⁺ [β2m]⁻ [TCR]⁻,
[anti-MUC1 CAR]⁺ [TGFβRII]⁻ [β2m]⁻ [TCR]⁻,
[anti-MUC1 CAR]⁺ [dnTGFβRII]⁺ [TGFβRII]⁻ [β2m]⁻ [TCR]⁻,
[anti-MUC1 CAR]⁺ [PD1]⁻,
[anti-MUC1 CAR]⁺ [TGFβRII]⁻ [PD1]⁻,
[anti-MUC1 CAR]⁺ [dnTGFβRII]⁺ [PD1]⁻,
[anti-MUC1 CAR]⁺ [dnTGFβRII]⁺ [TGFβRII]⁻ [PD1]⁻,
[anti-MUC1 CAR]⁺ [dnTGFβRII]⁺ [β2m]⁻ [PD1]⁻,
[anti-MUC1 CAR]⁺ [TGFβRII]⁻ [PD1]⁻ [TCR]⁻,
[anti-MUC1 CAR]⁺ [dnTGFβRII]⁺ [TGFβRII]⁻ [PD1]⁻ [TCR]⁻,
[anti-MUC1 CAR]⁺ [PD1]⁻ [β2m]⁻,
[anti-MUC1 CAR]⁺ [TGFβRII]⁻ [PD1]⁻ [β2m]⁻,
[anti-MUC1 CAR]⁺ [dnTGFβRII]⁺ [PD1]⁻ [β2m]⁻,
[anti-MUC1 CAR]⁺ [dnTGFβRII]⁺ [TGFβRII]⁻ [PD1]⁻ [β2m]⁻,
[anti-MUC1 CAR]⁺ [dnTGFβRII]⁺ [β2m]⁻ [PD1]⁻ [TCR]⁻,
[anti-MUC1 CAR]⁺ [TGFβRII]⁻ [PD1]⁻ [TCR]⁻ [β2m]⁻, or
[anti-MUC1 CAR]⁺ [dnTGFβRII]⁺ [TGFβRII]⁻ [PD1]⁻ [TCR]⁻ [β2m]⁻.

In even preferred embodiments, the above genotypes are combined with the over expression of IL-12 preferably via the insertion of IL-12a-2A-IL-12b construct and of HLAE as described herein and shown in FIG. 5, in order to obtain one of the following genotypes:
[anti-MUC1 CAR]⁺ [HLAE]⁺ [IL12]⁺,
[anti-MUC1 CAR]⁺ [dnTGFβRII]⁺ [HLAE]⁺ [IL12]⁺,
[anti-MUC1 CAR]⁺ [dnTGFβRII]⁺ [TCR]⁻ [HLAE]⁺ [IL12]⁺,
[anti-MUC1 CAR]⁺ [dnTGFβRII]⁺ [TGFβRII]⁻ [TCR]⁻ [HLAE]⁺ [IL12]⁺,
[anti-MUC1 CAR]⁺ [TGFβRII]⁻ [HLAE]⁺ [IL12]⁺,
[anti-MUC1 CAR]⁺ [TGFβRII]⁻ [TCR]⁻ [HLAE]⁺ [IL12]⁺,
[anti-MUC1 CAR]⁺ [β2m]⁻ [HLAE]⁺ [IL12]⁺,
[anti-MUC1 CAR]⁺ [dnTGFβRII]⁺ [β2m]⁻ [HLAE]⁺ [IL12]⁺,
[anti-MUC1 CAR]⁺ [TGFβRII]⁻ [β2m]⁻ [HLAE]⁺ [IL12]⁺,
[anti-MUC1 CAR]⁺ [dnTGFβRII]⁺ [β2m]⁻ [TCR]⁻ [HLAE]⁺ [IL12]⁺,
[anti-MUC1 CAR]⁺ [TGFβRII]⁻ [β2m]⁻ [TCR]⁻ [HLAE]⁺ [IL12]⁺,
[anti-MUC1 CAR]⁺ [dnTGFβRII]⁺ [TGFβRII]⁻ [β2m]⁻ [TCR]⁻ [HLAE]⁺ [IL12]⁺,
[anti-MUC1 CAR]⁺ [PD1]⁻ [HLAE]⁺ [IL12]⁺,
[anti-MUC1 CAR]⁺ [TGFβRII]⁻ [PD1]⁻ [HLAE]⁺ [IL12]⁺,
[anti-MUC1 CAR]⁺ [dnTGFβRII]⁺ [PD1]⁻ [HLAE]⁺ [IL12]⁺,
[anti-MUC1 CAR]⁺ [dnTGFβRII]⁺ [TGFβRII]⁻ [PD1]⁻ [HLAE]⁺ [IL12]⁺,
[anti-MUC1 CAR]⁺ [dnTGFβRII]⁺ [β2m]⁻ [PD1]⁻ [HLAE]⁺ [IL12]⁺,
[anti-MUC1 CAR]⁺ [TGFβRII]⁻ [PD1]⁻ [TCR]⁻ [HLAE]⁺ [IL12]⁺,
[anti-MUC1 CAR]⁺ [dnTGFβRII]⁺ [TGFβRII]-[PD1]⁻ [TCR]⁻ [HLAE]⁺ [IL12]⁺,
[anti-MUC1 CAR]⁺ [PD1]⁻ [β2m]⁻ [HLAE]⁺ [IL12]⁺,
[anti-MUC1 CAR]⁺ [TGFβRI]⁻ [PD1]⁻ [β2m]⁻ [HLAE]⁺ [IL12]⁺,
[anti-MUC1 CAR]⁺ [dnTGFβRII]⁺ [PD1]⁻ [β2m]⁻ [HLAE]⁺ [IL12]⁺,
[anti-MUC1 CAR]⁺ [dnTGFβRII]⁺ [TGFβRII]⁻ [PD1]⁻ [β2m]⁻ [HLAE]⁺ [IL12]⁺,
[anti-MUC1 CAR]⁺ [dnTGFβRII]⁺ [β2m]⁻ [PD1]⁻ [TCR]⁻ [HLAE]⁺ [IL12]⁺,
[anti-MUC1 CAR]⁺ [TGFβRII]⁻ [PD1]⁻ [TCR]⁻ [β2m]⁻ [HLAE]⁺ [IL12]⁺, or
[anti-MUC1 CAR]⁺ [dnTGFβRII]⁺ [TGFβRII]⁻ [PD1]⁻ [TCR]⁻ [β32m]-[HLAE]⁺ [IL12]⁺,

As mentioned before, the present invention is also particularly focused on the use of CAR positive cells in solid cancer treatments, which are made resistant to lymphodepleting agents, so that they can be combined with or preceded by lymphodepletion regimen for their use in allogeneic settings. Such cells preferably display the following genotypes:
with respect to anti-CD52 antibody partial or complete tolerance:
   [anti-MUC1 CAR]⁺ [CD52]⁻ [TCR]⁻,
   [anti-MUC1 CAR]⁺ [CD52]⁻ [TCR]⁻ [β2m]⁻,
   [anti-MUC1 CAR]⁺ [TGFβRII]⁻ [CD52]⁻ [TCR]⁻,
   [anti-MUC1 CAR]⁺ [TGFβRII]⁻ [CD52]⁻ [TCR]⁻ [β2m]⁻,
   [anti-MUC1 CAR]⁺ [dnTGFβRII]⁺ [CD52]⁻ [TCR]⁻,
   [anti-MUC1 CAR]⁺ [dnTGFβRII]⁺ [CD52]⁻ [TCR]⁻ [β2m]⁻,
with respect to purine analogues partial or complete tolerance:
   [anti-MUC1 CAR]⁺ [DCK]⁻ [TCR]⁻,
   [anti-MUC1 CAR]⁺ [DCK]⁻ [TCR]⁻ [β2m]⁻,
   [anti-MUC1 CAR]⁺ [TGFβRII]⁻ [DCK]⁻ [TCR]⁻,
   [anti-MUC1 CAR]⁺ [TGFβRII]⁻ [DCK]⁻ [TCR]⁻ [β2m]⁻,
   [anti-MUC1 CAR]⁺ [dnTGFβRII]⁺ [DCK]⁻ [TCR]⁻,
   [anti-MUC1 CAR]⁺ [dnTGFβRII]⁺ [DCK]⁻ [TCR]⁻ [β2m]⁻,
with respect to glucocorticoids partial or complete tolerance:
   [anti-MUC1 CAR]⁺ [GR]⁻ [TCR]⁻,
   [anti-MUC1 CAR]⁺ [GR]⁻ [TCR]⁻ [β2m]⁻,
   [anti-MUC1 CAR]⁺ [TGFβRII]⁻ [GR]⁻ [TCR]⁻,
   [anti-MUC1 CAR]⁺ [TGFβRII]⁻ [GR]⁻ [TCR]⁻ [β2m]⁻,
   [anti-MUC1 CAR]⁺ [dnTGFβRII]⁺ [GR]⁻ [TCR]⁻,
   [anti-MUC1 CAR]⁺ [dnTGFβRII]⁺ [GR]⁻ [TCR]⁻ [β2m]⁻,

### Further Improving Therapeutic Immune Cells by Expression of Transgenes at Inactivated Loci

The above preferred genotypes can be obtained by gene targeting integration, preferably at the PD1, TCR (TCRalpha and/or TCRbeta) or TGFβRII loci, but also at further selected loci as described here after.

By “gene targeting integration” is meant any known site-specific methods allowing to insert, replace or correct a genomic sequence into a living cell. Gene targeted integration usually involves the mechanisms of homologous gene recombination or NHEJ (Non homologous Ends Joining), which are enhanced by endonuclease sequence specific reagents, to result into insertion or replacement of at least one exogenous nucleotide, preferably a sequence of several nucleotides (i.e. polynucleotide), and more preferably a coding sequence at a predefined locus.

Expression of Further Transgenes which can Improve Immune Cells Potency Against Solid Tumors

The method of the present invention can be associated with other methods involving genetic transformations, such as a viral transduction, and also may be combined with other transgene expression not necessarily involving integration.

According to one aspect, the method according to the invention comprises the steps of introducing into an immune cell a mutation or polynucleotide coding sequence at an endogenous locus selected from:
a) polynucleotide sequence(s), which expression is(are) involved into reduction of glycolysis and calcium signaling in response to a low glucose condition, such as SERCA3 to increase calcium signaling, miR101 and mir26A to increase glycolysis, BCAT to mobilize glycolytic reserves; and/or
b) polynucleotide sequence(s), which expression up regulate(s) immune checkpoint proteins (e.g. TIM3, CEACAM, LAG3, TIGIT), such as IL27RA, STAT1, STAT3; and/or
c) polynucleotide sequence(s), which expression mediate(s) interaction with HLA-G, such as ILT2 or ILT4; and/or
d) polynucleotide sequence(s), which expression is(are) involved into the down regulation of T-cell proliferation such as SEMA7A, SHARPIN to reduce Treg proliferation, STAT1 to lower apoptosis, PEA15 to increase IL-2 secretion and RICTOR to favor CD8 memory differentiation; and/or
e) polynucleotide sequence(s), which expression is(are) involved into the down regulation of T-cell activation, such as mir21; and/or
f) polynucleotide sequence(s), which expression is(are) involved in signaling pathways responding to cytokines, such as JAK2 and AURKA; and/or
g) polynucleotide sequence(s), which expression is(are) involved in T-cell exhaustion, such as DNMT3, miRNA31, MT1A, MT2A, PTGER2;
preferably, by expressing into said cell a sequence-specific reagent that specifically targets said selected endogenous locus.

In further embodiments, the engineered immune cell as per the present invention can be further modified to obtain co-expression of an anti-MUC1 CAR in said cell with another exogenous genetic sequence selected from one encoding:
CRS inhibitor, such as is a mutated IL6Ra, sGP130 or IL18-BP;
Cytochrome(s) P450, CYP2D6-1, CYP2D6-2, CYP2C9, CYP3A4, CYP2C19 or CYP1A2, conferring hypersensitivity of said immune cells to a drug, such as cyclophosphamide and/or isophosphamide;
Dihydrofolate reductase (DHFR), inosine monophosphate dehydrogenase 2 (IMPDH2), calcineurin or methylguanine transferase (MGMT), mTORmut or Lckmut, conferring drug resistance;
Chemokine receptors, such as CCR2, CXCR2, or CXCR4; and/or
a secreted inhibitor of Tumor Associated Macrophages (TAM), such as a CCR2/CCL2 neutralization agent, to enhance the therapeutic activity of the immune cells;

Said transgene or exogenous polynucleotide sequence is preferably inserted so that its expression is placed under transcriptional control of at least one endogenous promoter present at one of said locus.

Targeting one locus as referred to above by performing gene integration is beneficial to further improve the potency of the therapeutic immune cells of the invention.

Examples of such exogenous sequences or transgenes that can be expressed or over-expressed at the selected loci are detailed hereafter:

### Expression of Further Transgenes Conferring Resistance to Drugs or Immune Depletion Agents

According to one aspect of the present method, the exogenous sequence that is integrated into the immune cells genomic locus encodes a molecule that confers resistance of said immune cells to a drug.

Examples of preferred exogenous sequences are variants of dihydrofolate reductase (DHFR) conferring resistance to folate analogs such as methotrexate, variants of inosine monophosphate dehydrogenase 2 (IMPDH2) conferring resistance to IMPDH inhibitors such as mycophenolic acid (MPA) or its prodrug mycophenolate mofetil (MMF), variants of calcineurin or methylguanine transferase (MGMT) conferring resistance to calcineurin inhibitor such as FK506 and/or CsA, variants of mTOR such as mTORmut conferring resistance to rapamycin) and variants of Lck, such as Lckmut conferring resistance to Imatinib and Gleevec.

The term “drug” is used herein as referring to a compound or a derivative thereof, preferably a standard chemotherapy agent that is generally used for interacting with a cancer cell, thereby reducing the proliferative or living status of the cell. Examples of chemotherapeutic agents include, but are not limited to, alkylating agents (e.g., cyclophosphamide, ifosamide), metabolic antagonists (e.g., purine nucleoside antimetabolite such as clofarabine, fludarabine or 2′-deoxyadenosine, methotrexate (MTX), 5-fluorouracil or derivatives thereof), antitumor antibiotics (e.g., mitomycin, adriamycin), plant-derived antitumor agents (e.g., vincristine, vindesine, Taxol), cisplatin, carboplatin, etoposide, and the like. Such agents may further include, but are not limited to, the anti-cancer agents TRIMETHOTRIXATE™ (TMTX), TEMOZOLOMIDE™, RALTRITREXED™, S-(4-Nitrobenzyl)-6-thioinosine (NBMPR),6-benzyguanidine (6-BG), bis-chloronitrosourea (BCNU) and CAMPTOTHECIN™, or a therapeutic derivative of any thereof.

As used herein, an immune cell is made “resistant or tolerant” to a drug when said cell, or population of cells is modified so that it can proliferate, at least in-vitro, in a culture medium containing half maximal inhibitory concentration (IC50) of said drug (said IC50 being determined with respect to an unmodified cell(s) or population of cells).

In a particular embodiment, said drug resistance can be conferred to the immune cells by the expression of at least one “drug resistance coding sequence”. Said drug resistance coding sequence refers to a nucleic acid sequence that confers “resistance” to an agent, such as one of the chemotherapeutic agents referred to above. A drug resistance coding sequence of the invention can encode resistance to anti-metabolite, methotrexate, vinblastine, cisplatin, alkylating agents, anthracyclines, cytotoxic antibiotics, anti-immunophilins, their analogs or derivatives, and the like (Takebe, N., S. C. Zhao, et al. (2001) “Generation of dual resistance to 4-hydroperoxycyclophosphamide and methotrexate by retroviral transfer of the human aldehyde dehydrogenase class 1 gene and a mutated dihydrofolate reductase gene”. Mol. Ther. 3(1): 88-96), (Zielske, S. P., J. S. Reese, et al. (2003) “In vivo selection of MGMT(P140K) lentivirus-transduced human NOD/SCID repopulating cells without pretransplant irradiation conditioning.” J. Clin. Invest. 112(10): 1561-70) (Nivens, M. C., T. Felder, et al. (2004) “Engineered resistance to camptothecin and antifolates by retroviral coexpression of tyrosyl DNA phosphodiesterase-I and thymidylate synthase” Cancer Chemother Pharmacol 53(2): 107-15), (Bardenheuer, W., K. Lehmberg, et al. (2005). “Resistance to cytarabine and gemcitabine and in vitro selection of transduced cells after retroviral expression of cytidine deaminase in human hematopoietic progenitor cells”. Leukemia 19(12): 2281-8), (Kushman, M. E., S. L. Kabler, et al. (2007) “Expression of human glutathione S-transferase P1 confers resistance to benzo[a]pyrene or benzo[a]pyrene-7,8-dihydrodiol mutagenesis, macromolecular alkylation and formation of stable N2-Gua-BPDE adducts in stably transfected V79MZ cells co-expressing hCYP1A1” Carcinogenesis 28(1): 207-14).

The expression of such drug resistance exogenous sequences in the immune cells as per the present invention more particularly allows the use of said immune cells in cell therapy treatment schemes where cell therapy is combined with chemotherapy or into patients previously treated with these drugs.

Several drug resistance coding sequences have been identified that can potentially be used to confer drug resistance according to the invention. One example of drug resistance coding sequence can be for instance a mutant or modified form of Dihydrofolate reductase (DHFR). DHFR is an enzyme involved in regulating the amount of tetrahydrofolate in the cell and is essential to DNA synthesis. Folate analogs such as methotrexate (MTX) inhibit DHFR and are thus used as anti-neoplastic agents in clinic. Different mutant forms of DHFR which have increased resistance to inhibition by anti-folates used in therapy have been described. In a particular embodiment, the drug resistance coding sequence according to the present invention can be a nucleic acid sequence encoding a mutant form of human wild type DHFR (GenBank: AAH71996.1), which comprises at least one mutation conferring resistance to an anti-folate treatment, such as methotrexate. In particular embodiment, mutant form of DHFR comprises at least one mutated amino acid at position G15, L22, F31 or F34, preferably at positions L22 or F31 (Schweitzer et al. (1990) “Dihydrofolate reductase as a therapeutic target” Faseb J 4(8): 2441-52; International application WO94/24277; and U.S. Pat. No. 6,642,043). In a particular embodiment, said DHFR mutant form comprises two mutated amino acids at position L22 and F31. Correspondence of amino acid positions described herein is frequently expressed in terms of the positions of the amino acids of the form of wild-type DHFR polypeptide. In a particular embodiment, the serine residue at position 15 is preferably replaced with a tryptophan residue. In another particular embodiment, the leucine residue at position 22 is preferably replaced with an amino acid which will disrupt binding of the mutant DHFR to antifolates, preferably with uncharged amino acid residues such as phenylalanine or tyrosine. In another particular embodiment, the phenylalanine residue at positions 31 or 34 is preferably replaced with a small hydrophilic amino acid such as alanine, serine or glycine.

Another example of drug resistance coding sequence can also be a mutant or modified form of ionisine-5′-monophosphate dehydrogenase II (IMPDH2), a rate-limiting enzyme in the de novo synthesis of guanosine nucleotides. The mutant or modified form of IMPDH2 is a IMPDH inhibitor resistance gene. IMPDH inhibitors can be mycophenolic acid (MPA) or its prodrug mycophenolate mofetil (MMF). The mutant IMPDH2 can comprises at least one, preferably two mutations in the MAP binding site of the wild type human IMPDH2 (Genebank: NP_000875.2) leading to a significantly increased resistance to IMPDH inhibitor. Mutations in these variants are preferably at positions T333 and/or S351 (Yam, P., M. Jensen, et al. (2006) “Ex vivo selection and expansion of cells based on expression of a mutated inosine monophosphate dehydrogenase 2 after HIV vector transduction: effects on lymphocytes, monocytes, and CD34+ stem cells” Mol. Ther. 14(2): 236-44)(Jonnalagadda, M., et al. (2013) “Engineering human T cells for resistance to methotrexate and mycophenolate mofetil as an in vivo cell selection strategy.” PLoS One 8(6): e65519).

Another drug resistance coding sequence is the mutant form of calcineurin. Calcineurin (PP2B-NCBI: ACX34092.1) is an ubiquitously expressed serine/threonine protein phosphatase that is involved in many biological processes and which is central to T-cell activation. Calcineurin is a heterodimer composed of a catalytic subunit (CnA; three isoforms) and a regulatory subunit (CnB; two isoforms). After engagement of the T-cell receptor, calcineurin dephosphorylates the transcription factor NFAT, allowing it to translocate to the nucleus and active key target gene such as IL2. FK506 in complex with FKBP12, or cyclosporine A (CsA) in complex with CyPA block NFAT access to calcineurin's active site, preventing its dephosphorylation and thereby inhibiting T-cell activation (Brewin et al. (2009) “Generation of EBV-specific cytotoxic T cells that are resistant to calcineurin inhibitors for the treatment of posttransplantation lymphoproliferative disease” Blood 114(23): 4792-803). In a particular embodiment, said mutant form can comprise at least one mutated amino acid of the wild type calcineurin heterodimer a at positions: V314, Y341, M347, T351, W352, L354, K360, preferably double mutations at positions T351 and L354 or V314 and Y341. In a particular embodiment, the valine residue at position 341 can be replaced with a lysine or an arginine residue, the tyrosine residue at position 341 can be replaced with a phenylalanine residue; the methionine at position 347 can be replaced with the glutamic acid, arginine or tryptophane residue; the threonine at position 351 can be replaced with the glutamic acid residue; the tryptophane residue at position 352 can be replaced with a cysteine, glutamic acid or alanine residue, the serine at position 353 can be replaced with the histidine or asparagines residue, the leucine at position 354 can be replaced with an alanine residue; the lysine at position 360 can be replaced with an alanine or phenylalanine residue. In another particular embodiment, said mutant form can comprise at least one mutated amino acid of the wild type calcineurin heterodimer b at positions: V120, N123, L124 or K125, preferably double mutations at positions L124 and K125. In a particular embodiment, the valine at position 120 can be replaced with a serine, an aspartic acid, phenylalanine or leucine residue; the asparagines at position 123 can be replaced with a tryptophan, lysine, phenylalanine, arginine, histidine or serine; the leucine at position 124 can be replaced with a threonine residue; the lysine at position 125 can be replaced with an alanine, a glutamic acid, tryptophan, or two residues such as leucine-arginine or isoleucine-glutamic acid can be added after the lysine at position 125 in the amino acid sequence. Correspondence of amino acid positions described herein is frequently expressed in terms of the positions of the amino acids of the form of wild-type human calcineurin heterodimer b polypeptide (NCBI: ACX34095.1).

Another drug resistance coding sequence is 0(6)-methylguanine methyltransferase (MGMT-UniProtKB: P16455) encoding human alkyl guanine transferase (hAGT). AGT is a DNA repair protein that confers resistance to the cytotoxic effects of alkylating agents, such as nitrosoureas and temozolomide (TMZ). 6-benzylguanine (6-BG) is an inhibitor of AGT that potentiates nitrosourea toxicity and is co-administered with TMZ to potentiate the cytotoxic effects of this agent. Several mutant forms of MGMT that encode variants of AGT are highly resistant to inactivation by 6-BG, but retain their ability to repair DNA damage (Maze, R. et al. (1999) “Retroviral-mediated expression of the P140A, but not P140A/G156A, mutant form of 06-methylguanine DNA methyltransferase protects hematopoietic cells against 06-benzylguanine sensitization to chloroethylnitrosourea treatment” J. Pharmacol. Exp. Ther. 290(3): 1467-74). In a particular embodiment, AGT mutant form can comprise a mutated amino acid of the wild type AGT position P140. In a preferred embodiment, said proline at position 140 is replaced with a lysine residue.

Another drug resistance coding sequence can be multidrug resistance protein (MDR1) gene. This gene encodes a membrane glycoprotein, known as P-glycoprotein (P-GP) involved in the transport of metabolic byproducts across the cell membrane. The P-Gp protein displays broad specificity towards several structurally unrelated chemotherapy agents. Thus, drug resistance can be conferred to cells by the expression of nucleic acid sequence that encodes MDR-1 (Genebank NP_000918).

Another drug resistance coding sequence can contribute to the production of cytotoxic antibiotics, such as those from ble or mcrA genes. Ectopic expression of ble gene or mcrA in an immune cell gives a selective advantage when exposed to the respective chemotherapeutic agents bleomycine and mitomycin C (Belcourt, M. F. (1999) “Mitomycin resistance in mammalian cells expressing the bacterial mitomycin C resistance protein MCRA”. PNAS. 96(18):10489-94).

Another drug resistance coding sequence can come from genes encoded mutated version of drug targets, such as mutated variants of mTOR (mTOR mut) conferring resistance to rapamycin such as described by Lorenz M. C. et al. (1995) “TOR Mutations Confer Rapamycin Resistance by Preventing Interaction with FKBP12-Rapamycin” The Journal of Biological Chemistry 270, 27531-27537, or certain mutated variants of Lck (Lckmut) conferring resistance to Gleevec as described by Lee K. C. et al. (2010) “Lck is a key target of imatinib and dasatinib in T-cell activation”, Leukemia, 24: 896-900.

As described above, the genetic modification step of the method can comprise a step of introduction into cells of an exogeneous nucleic acid comprising at least a sequence encoding the drug resistance coding sequence and a portion of an endogenous gene such that homologous recombination occurs between the endogenous gene and the exogeneous nucleic acid. In a particular embodiment, said endogenous gene can be the wild type “drug resistance” gene, such that after homologous recombination, the wild type gene is replaced by the mutant form of the gene which confers resistance to the drug.

### Expression of Transgene Enhancing Persistence of the Immune Cells In-Vivo

According to one aspect of the present method, the exogenous sequence that is integrated into the immune cells genomic locus encodes a molecule that enhances persistence of the immune cells, especially in-vivo persistence in a tumor environment.

By “enhancing persistence” is meant extending the survival of the immune cells in terms of life span, especially once the engineered immune cells are injected into the patient. For instance, persistence is enhanced, if the mean survival of the modified cells is significantly longer than that of non-modified cells, by at least 10%, preferably 20%, more preferably 30%, even more preferably 50%.

This especially relevant when the immune cells are allogeneic. This may be done by creating a local immune protection by introducing coding sequences that ectopically express and/or secrete immunosuppressive polypeptides at, or through, the cell membrane. A various panel of such polypeptides in particular antagonists of immune checkpoints, immunosuppressive peptides derived from viral envelope or NKG2D ligand can enhance persistence and/or an engraftment of allogeneic immune cells into patients.

According to one embodiment, the immunosuppressive polypeptide to be encoded by said exogenous coding sequence is a ligand of Cytotoxic T-Lymphocyte Antigen 4 (CTLA-4 also known as CD152, GenBank accession number AF414120.1). Said ligand polypeptide is preferably an anti-CTLA-4 immunoglobulin, such as CTLA-4a Ig and CTLA-4b Ig or a functional variant thereof.

According to one embodiment, the immunosuppressive polypeptide to be encoded by said exogenous coding sequence is an antagonist of PD1, such as PD-L1 (other names: CD274, Programmed cell death 1 ligand; ref. UniProt for the human polypeptide sequence Q9NZQ7), which encodes a type I transmembrane protein of 290 amino acids consisting of a Ig V-like domain, a Ig C-like domain, a hydrophobic transmembrane domain and a cytoplasmic tail of 30 amino acids. Such membrane-bound form of PD-L1 ligand is meant in the present invention under a native form (wild-type) or under a truncated form such as, for instance, by removing the intracellular domain, or with one or more mutation(s) (Wang S et al., 2003, J Exp Med. 2003; 197(9): 1083-1091). Of note, PD1 is not considered as being a membrane-bound form of PD-L1 ligand according to the present invention. According to another embodiment, said immunosuppressive polypeptide is under a secreted form. Such recombinant secreted PD-L1 (or soluble PD-L1) may be generated by fusing the extracellular domain of PD-L1 to the Fc portion of an immunoglobulin (Haile S T et al., 2014, Cancer Immunol. Res. 2(7): 610-615; Song M Y et al., 2015, Gut. 64(2):260-71). This recombinant PD-L1 can neutralize PD-1 and abrogate PD-1-mediated T-cell inhibition. PD-L1 ligand may be co-expressed with CTLA4 Ig for an even enhanced persistence of both.

According to another embodiment, the exogenous sequence encodes a non-human MHC homolog, especially a viral MHC homolog, or a chimeric β2m polypeptide such as described by Margalit A. et al. (2003) “Chimeric β2 microglobulin/CD3ζ polypeptides expressed in T cells convert MHC class I peptide ligands into T cell activation receptors: a potential tool for specific targeting of pathogenic CD8+ T cells” Int. Immunol. 15 (11): 1379-1387.

According to one embodiment, the exogenous sequence encodes NKG2D ligand. Some viruses such as cytomegaloviruses have acquired mechanisms to avoid NK cell mediate immune surveillance and interfere with the NKG2D pathway by secreting a protein able to bind NKG2D ligands and prevent their surface expression (Welte, S. A et al. (2003) “Selective intracellular retention of virally induced NKG2D ligands by the human cytomegalovirus UL16 glycoprotein”. Eur. J. Immunol., 33, 194-203). In tumors cells, some mechanisms have evolved to evade NKG2D response by secreting NKG2D ligands such as ULBP2, MICB or MICA (Salih H R, Antropius H, Gieseke F, Lutz S Z, Kanz L, et al. (2003) Functional expression and release of ligands for the activating immunoreceptor NKG2D in leukemia. Blood 102: 1389-1396).

According to one embodiment, the exogenous sequence encodes a cytokine receptor, such as an IL-12 receptor. IL-12 is a well known activator of immune cells activation (Curtis J. H. (2008) “IL-12 Produced by Dendritic Cells Augments CD8+ T Cell Activation through the Production of the Chemokines CCL1 and CCL171”. The Journal of Immunology. 181 (12): 8576-8584.

According to one embodiment the exogenous sequence encodes an antibody that is directed against inhibitory peptides or proteins. Said antibody is preferably be secreted under soluble form by the immune cells. Nanobodies from shark and camels are advantageous in this respect, as they are structured as single chain antibodies (Muyldermans S. (2013) “Nanobodies: Natural Single-Domain Antibodies” Annual Review of Biochemistry 82: 775-797). Same are also deemed more easily to fuse with secretion signal polypeptides and with soluble hydrophilic domains.

The different aspects developed above to enhance persistence of the cells are particularly preferred, when the exogenous coding sequence is introduced by disrupting an endogenous gene encoding β2m or another MHC component, as detailed further on.

### Expression of Transgenes Enhancing the Therapeutic Activity of Immune Cells

According to one aspect of the present method, the exogenous sequence that is integrated into the immune cells genomic locus encodes a molecule that enhances the therapeutic activity of the immune cells.

By “enhancing the therapeutic activity” is meant that the immune cells, or population of cells, engineered according to the present invention, become more aggressive than non-engineered cells or population of cells with respect to a selected type of target cells. Said target cells consists of a defined type of cells, or population of cells, preferably characterized by common surface marker(s). In the present specification, “therapeutic potential” reflects the therapeutic activity, as measured through in-vitro experiments. In general sensitive cancer cell lines, such as Daudi cells, are used to assess whether the immune cells are more or less active towards said cells by performing cell lysis or growth reduction measurements. This can also be assessed by measuring levels of degranulation of immune cells or chemokines and cytokines production. Experiments can also be performed in mice with injection of tumor cells, and by monitoring the resulting tumor expansion. Enhancement of activity is deemed significant when the number of developing cells in these experiments is reduced by the immune cells by more than 10%, preferably more than 20%, more preferably more than 30%, even more preferably by more than 50%.

According to one aspect of the invention, said exogenous sequence encodes a chemokine or a cytokine, such as IL-12. It is particularly advantageous to express IL-12 as this cytokine is extensively referred to in the literature as promoting immune cell activation (Colombo M. P. et al. (2002) “Interleukin-12 in anti-tumor immunity and immunotherapy” Cytokine Growth Factor Rev. 13(2):155-68).

According to a preferred aspect of the invention the exogenous coding sequence encodes or promote secreted factors that act on other populations of immune cells, such as T-regulatory cells, to alleviate their inhibitory effect on said immune cells.

According to one aspect of the invention, said exogenous sequence encodes an inhibitor of regulatory T-cell activity is a polypeptide inhibitor of forkhead/winged helix transcription factor 3 (FoxP3), and more preferably is a cell-penetrating peptide inhibitor of FoxP3, such as that referred as P60 (Casares N. et al. (2010) “A peptide inhibitor of FoxP3 impairs regulatory T cell activity and improves vaccine efficacy in mice.” J Immunol 185(9):5150-9).

By “inhibitor of regulatory T-cells activity” is meant a molecule or precursor of said molecule secreted by the T-cells and which allow T-cells to escape the down regulation activity exercised by the regulatory T-cells thereon. In general, such inhibitor of regulatory T-cell activity has the effect of reducing FoxP3 transcriptional activity in said cells.

According to one aspect of the invention, said exogenous sequence encodes a secreted inhibitor of Tumor Associated Macrophages (TAM), such as a CCR2/CCL2 neutralization agent. Tumor-associated macrophages (TAMs) are critical modulators of the tumor microenvironment. Clinicopathological studies have suggested that TAM accumulation in tumors correlates with a poor clinical outcome. Consistent with that evidence, experimental and animal studies have supported the notion that TAMs can provide a favorable microenvironment to promote tumor development and progression. (Theerawut C. et al. (2014) “Tumor-Associated Macrophages as Major Players in the Tumor Microenvironment” Cancers (Basel) 6(3): 1670-1690). Chemokine ligand 2 (CCL2), also called monocyte chemoattractant protein 1 (MCP1—NCBI NP_002973.1), is a small cytokine that belongs to the CC chemokine family, secreted by macrophages, that produces chemoattraction on monocytes, lymphocytes and basophils. CCR2 (C-C chemokine receptor type 2—NCBI NP_001116513.2), is the receptor of CCL2.

Although the coding sequence which is inserted at said locus generally encodes polypeptide(s) improving the therapeutic potential of the engineered immune cells, the inserted sequence can also be a nucleic acid able to direct or repress expression of other genes, such as interference RNAs or guide-RNAs. The polypeptides encoded by the inserted sequence may act directly or indirectly, such as signal transducers or transcriptional regulators.

### Engineered Immune Cells and Populations of Immune Cells

The present invention is also drawn to the variety of engineered immune cells obtainable according to one of the method described herein, under isolated form, or as part of populations of cells.
According to a preferred aspect of the invention the engineered cells are primary immune cells, such as NK cells or T-cells, which are generally part of populations of cells that may involve different types of cells. In general, population deriving from patients or donors isolated by leukapheresis from PBMC (peripheral blood mononuclear cells).

The present invention encompasses immune cells comprising any combinations of the different exogenous coding sequences and gene inactivation, which have been respectively and independently described above. Among these combinations are particularly preferred those combining the expression of a CAR under the transcriptional control of an endogenous promoter that is active during immune cell activation, in particular one promoter present at one TCR locus, in particular a TCRalpha promoter.

Another preferred combination is the insertion of an exogenous sequence encoding a CAR or one of its constituents under the transcription control of the hypoxia-inducible factor 1 gene promoter (Uniprot: Q16665).

The invention is also drawn to a pharmaceutical composition comprising an engineered primary immune cell or immune cell population as previously described for the treatment of infection or cancer, and to a method for treating a patient in need thereof, wherein said method comprises:
preparing a population of engineered primary immune cells according to the method of the invention as previously described;
optionally, purifying or sorting said engineered primary immune cells;
activating said population of engineered primary immune cells upon or after infusion of said cells into said patient.

The present invention has also for object the engineered immune cells resulting from the above methods, wherein said cells comprise an exogenous polynucleotide or an expression vector as referred to herein, especially for the expression of an anti-MUC1 CAR at its cell surface. Such engineered immune cells are preferably T-cells or NK cells, which are derived from primary cells or differentiated from stem cells, such as PS cells.

According to certain embodiments, the expression of TCR is reduced or suppressed in said immune cells by inactivation of at least one gene encoding TCRalpha or TCRbeta. This by a rare-cutting endonuclease.

According to certain embodiments, the polynucleotide encoding the anti-MUC1 CAR can be integrated at an endogenous locus under transcriptional control of an endogenous promoter, preferably at the TCRalpha or TCRbeta locus.

According to certain embodiments, immune cells can be further mutated to confer resistance to at least one immune suppressive drug, such as an anti-CD52 antibody.

According to certain embodiments, immune cells can be further mutated to confer resistance to at least one chemotherapy drug, in particular a purine analogue drug.

According to preferred embodiments, an engineered immune cell of the present invention has been mutated to improve its persistence or its lifespan into the patient, in particular into a gene encoding MHCI component(s) such as HLA or B2m.

According to preferred embodiments, an engineered immune cell of the present invention can be mutated to improve its CAR-dependent immune activation, in particular to reduce or suppress the expression of immune checkpoint proteins and/or receptors thereof.

According to certain embodiments, the anti-MUC1 CAR can be co-expressed in said cell with another exogenous genetic sequence encoding an inhibitor or decoy of TGFbeta receptor, such as a dominant negative TGFbeta receptor (dnTGFβRII) having at least 80% polypeptide sequence identity with SEQ ID NO:59. This expression can be obtained by introducing into the cell an exogenous polynucleotide comprising a first polynucleotide sequence encoding said anti-MUC1 CAR, a second polynucleotide encoding a 2A self-cleaving peptide, and a third polynucleotide encoding said dominant negative TGFbeta receptor.

Alternatively or supplementally, the engineered immune cell can have at least one TGFbeta receptor gene expression reduced or inactivated.

According to some further embodiments, the anti-MUC1 chimeric antigen receptor (CAR) can be co-expressed in said cell with another exogenous genetic sequence selected from one encoding:
NK cell inhibitor, such as HLAG, HLAE or ULBP1;
CRS inhibitor, such as is a mutated IL6Ra, sGP130 or IL18-BP; or
Cytochrome(s) P450, CYP2D6-1, CYP2D6-2, CYP2C9, CYP3A4, CYP2C19 or CYP1A2, conferring hypersensitivity of said immune cells to a drug, such as cyclophosphamide and/or isophosphamide,
Dihydrofolate reductase (DHFR), inosine monophosphate dehydrogenase 2 (IMPDH2), calcineurin or methylguanine transferase (MGMT), mTORmut or Lckmut, conferring drug resistance;
Chemokine or a cytokine, such as IL-2, IL-12 and IL-15;
Hyaluronidase, such as HYAL1, HYAL2 and SPAM1;
Chemokine receptors, such as CCR2, CXCR2, or CXCR4;
a secreted inhibitor of Tumor Associated Macrophages (TAM), such as a CCR2/CCL2 neutralization agent, to enhance the therapeutic activity of the immune cells; and/or
metabolic enzymes such as: glucose phosphate isomerase 1 (GPI1), lactate deshydrogenase (LDHA) and/or phosphoenolpyruvate carboxykinase 1 (PCK1).

### Dual Anti-MESOTHELIN/Anti-MUC1 CAR Immune Cells

As a further embodiment, the present invention also features immune cells or population of immune cells expressing both an anti-MUC1 CAR as previously described and an anti-MESOTHELIN CAR. This specific combination of CARs has significant advantage for treating solid tumors that are both MESOTHELIN and MUC1 positive. Targeting these two genetically unrelated antigens reduces chances for antigen escape. The inventors have found that this combination confers to the populations of cells of the present invention an extra capacity over solid tumors in-vivo. This also allows extending antitumor activity coverage, especially in heterogenous solid tumors where different types of tumorigenic cells can coexist. For instance, dual CAR immune cells expressing anti-MESOTHELIN CAR and anti-MUC1 CAR positive cells can kill either MESOTHELIN positive or tMUC1 positive cells, where MUC1 CAR alone could not.

Both anti-MUC1 CAR and anti-MESOTHELIN CAR can be co-expressed in the immune cells engineered by the methods as previously described, and thus can comprise any of the genetic attributes referred to in the present application such as reduced expression of TCR, B2M and PD1. For instance, the immune cells can be transduced with a viral vector comprising a polynucleotide construct including sequences encoding either and anti-MUC1 CAR or anti-MESOTHELIN CAR, or also a polynucleotide encoding both coding sequences linked by a 2A polypeptide. Alternatively, therapeutic population of cells can be obtained by mixing immune cells independently expressing anti-MUC1 CAR or anti-MESOTHELIN CAR.

The present invention thus encompasses therapeutic populations of engineered immune cells comprising a mix of cells expressing either an anti-MUC1 CAR or anti-MESOTHELIN CAR or both CARs, for their use in anti-cancer therapy, especially for the treatment of breast, cervical, endometrial, ovarian, pancreatic, stomach and lung cancers.

According to preferred embodiments, the mesothelin specific chimeric antigen receptors (anti-MESO CAR) is one that presents a structure comprising typically:
an extracellular ligand binding-domain comprising VH and VL from a monoclonal anti-mesothelin antibody;
a transmembrane domain; and
a cytoplasmic domain comprising a CD3 zeta signalling domain and a co-stimulatory domain.

According to preferred embodiments, the mesothelin specific chimeric antigen receptors (anti-MESO CAR) is one directed against human mesothelin (MSLN_human referred to as Q13421 in the Uniprot database), and more particularly specific mesothelin's polypeptide region represented by SEQ ID NO:209, which is presented at the surface of malignant cells. Efficient CARs have been developed by the applicant against this antigen region, in particular one comprising an extra cellular ligand binding-domain, which comprises VH and VL chains having at least 80%, preferably at least 90%, more preferably at least 95%, and even more preferably at least 99% sequence identity respectively with SEQ ID NO:210 (Meso1-VH) and SEQ ID NO:211 (Meso1-VL).

The extracellular ligand binding domain of the anti-MESO CARs preferably comprises one or several scFv segment from the antibody referred to as meso1, and more particularly the CDRs therefrom including SEQ ID NO:216, 217, 218, 219, 220 and/or 221.

According to a preferred aspect, said extracellular ligand binding domain of said CARs comprise:
a variable heavy VH chain comprising CDRs from the antibody meso1 having respectively at least 90% identity with SEQ ID NO:3 (CDRH1-Meso1), SEQ ID NO:4 (CDRH2-meso1) and/or SEQ ID NO:5 (CDRH3-meso1), and
a variable heavy VL chain comprising CDRs from the antibody Meso1 having respectively at least 90% identity with SEQ ID NO:6 (CDRL1-meso1), SEQ ID NO:7 (CDRL2-meso1) and/or SEQ ID NO:8 (CDRL3-meso1).

One preferred anti-MESOTHELIN CAR to be used in combination with an anti-MUC1 CAR as per the present invention is the one referred to as MESO1 CAR, which has at least 75%, preferably at least 80%, more preferably at least 90%, more preferably at least 95%, and even more preferably at least 99% identity with SEQ ID NO:222.

Other anti-MESO CARs can also be expressed as part of the present invention, such as for example MESO2 and P4-R2 or any functional variant thereof comprising ScFv sequences at least 80%, preferably at least 90%, more preferably at least 95%, and even more preferably at least 99% identity with the polypeptide sequences referred to in Table 12 and 13.

TABLE 12
Amino acid sequences of exemplary
binding domains of anti-MESO CARs

| ScFv | SEQ ID | Raw amino |
|---|---|---|
| sequences | # | acid sequence |
| MESO1 heavy | SEQ ID | QVQLQESGPGLVKPSETLSL |
| chain variable | NO. 210 | TCTVSGGSISSYYWSWIRQP |
| region | | PGKGLEWIGYIYYSGSTNYN |
| | | PSLKSRVTISVDTSKNQFSL |
| | | KLSSVTAADTAVYYCARVDY |
| | | KAFDIWGQGTMVTVSS |
| MESO1 light | SEQ ID | DIQMTQSPSSLSASVGDRVT |
| chain variable | NO. 211 | ITCRASQGIRNDLHWYQQKP |
| region | | GKAPKRLIYAASSLQSGVPS |
| | | RFSGSGSGTEFTLTISSLQP |
| | | EDFATYYCLQHYSYPWTFGQ |
| | | GTKVEIK |
| MESO2 heavy | SEQ ID | QVQLVQSGAEVKKPGESLKI |
| chain variable | NO: 212 | SCKGSGYSFTNYWIGWVRQM |
| region | | PGKGLEWMGVIMPSDSYTRY |
| | | SPSFQGQVTISADKSISTAY |
| | | LQWSSLKASDTAMYYCARYG |
| | | HGMYGGALDVWGQGTLVTVS |
| | | S |
| MESO2 light | SEQ ID | DIVLTQSPATLSLSPGERAT |
| chain variable | NO: 213 | LSCRASQSVRSSRLAWYQQK |
| region | | PGQAPRLLIYGASKRATGVP |
| | | ARFSGSGSGTDFTLTISSLE |
| | | PEDFAVYYCQQYSHDPSGTF |
| | | GQGTKVEIKRT |
| P4 heavy chain | SEQ ID | QVQLQQSGPGLVTPSQTLSL |
| variable region | NO: 214 | TCAISGDSVSSNSATWNWIR |
| | | QSPSRGLEWLGRTYYRSKWY |
| | | NDYAVSVKSRMSINPDTSKN |
| | | QFSLQLNSVTPEDTAVYYCA |
| | | RGMMTYYYGMDVWGQGTTVT |
| | | VSSGILG |
| P4 light chain | SEQ ID | QPVLTQSSSLSASPGASASL |
| variable region | NO: 215 | TCTLRSGINVGPYRIYWYQQ |
| | | KPGSPPQYLLNYKSDSDKQQ |
| | | GSGVPSRFSGSKDASANAGV |
| | | LLISGLRSEDEADYYCMIWH |
| | | SSAAVFGGGTQLTVLS |
| CDRH1-Meso1 | SEQ ID | SYYWS |
| | NO: 216 | |
| CDRH2-Meso1 | SEQ ID | YIYYSGSTNYNPSLKS |
| | NO: 217 | |
| CDRH3-Meso1 | SEQ ID | VDYKAFDI |
| | NO: 218 | |
| CDRL1-Meso1 | SEQ ID | RASQGIRNDLH |
| | NO: 219 | |
| CDRL2-Meso1 | SEQ ID | AASSLQS |
| | NO: 220 | |
| CDRL3-Meso1 | SEQ ID | LQHYSYPWT |
| | NO: 221 | |

TABLE 13
Examples of amino acid sequences of P4-R2, Meso1-R2, Meso1 and MESO2-R2 CARs.

| | Domains | | | | | | | | |
|---|---|---|---|---|---|---|---|---|---|
| CAR | signal | | | | R2 | | | | |
| name | peptide | | | | suicide | CD8α | CD8α | 4-1BB | CD3ζ |
| Full | (optional) | VH | linker | VL | switch | hinge | TM | IC | IC |
| P4-R2 | SEQ ID | SEQ ID | SEQ ID | SEQ ID | SEQ ID | SEQ ID | SEQ ID | SEQ ID | SEQ ID |
| | NO: 2 | NO: 214 | NO: 8 | NO: 215 | NO: 3 | NO: 4 | NO: 5 | NO: 6 | NO: 7 |
| Meso1- | SEQ ID | SEQ ID | SEQ ID | SEQ ID | SEQ ID | SEQ ID | SEQ ID | SEQ ID | SEQ ID |
| R2 | NO: 2 | NO: 210 | NO: 8 | NO: 211 | NO: 3 | NO: 4 | NO: 5 | NO: 6 | NO: 7 |
| Meso1 | SEQ ID | SEQ ID | SEQ ID | SEQ ID | None | SEQ ID | SEQ ID | SEQ ID | SEQ ID |
| CAR | NO: 2 | NO: 210 | NO: 8 | NO: 211 | | NO: 4 | NO: 5 | NO: 6 | NO: 7 |
| (SEQ ID | | | | | | | | | |
| NO: 222) | | | | | | | | | |
| MESO2- | SEQ ID | SEQ ID | SEQ ID | SEQ ID | SEQ ID | SEQ ID | SEQ ID | SEQ ID | SEQ ID |
| R2 | NO: 2 | NO: 212 | NO: 8 | NO: 213 | \|NO: 3 | NO: 4 | NO: 5 | NO: 6 | NO: 7 |

### Activation and Expansion of T-Cells

Whether prior to or after genetic modification, the immune cells according to the present invention can be activated or expanded, even if they can activate or proliferate independently of antigen binding mechanisms. T-cells, in particular, can be activated and expanded using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005. T-cells can be expanded in vitro or in vivo. T cells are generally expanded by contact with an agent that stimulates a CD3 TCR complex and a co-stimulatory molecule on the surface of the T-cells to create an activation signal for the T-cell. For example, chemicals such as calcium ionophore A23187, phorbol 12-myristate 13-acetate (PMA), or mitogenic lectins like phytohemagglutinin (PHA) can be used to create an activation signal for the T-cell.

As non-limiting examples, T cell populations may be stimulated in vitro such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. Conditions appropriate for T cell culture include an appropriate media (e.g., Minimal Essential Media or RPMI Media 1640 or, X-vivo 5, (Lonza)) that may contain factors necessary for proliferation and viability, including serum (e.g., fetal bovine or human serum), interleukin-2 (IL-2), insulin, IFN-g, IL-4, IL-7, GM-CSF, IL-10, IL-2, IL-15, TGFp, and TNF- or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, A1M-V, DMEM, MEM, a-MEM, F-12, X-Vivo 1, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% C02). T cells that have been exposed to varied stimulation times may exhibit different characteristics.

In another particular embodiment, said cells can be expanded by co-culturing with tissue or cells. Said cells can also be expanded in vivo, for example in the subject's blood after administrating said cell into the subject.

### Therapeutic Compositions and Applications

The method of the present invention described above allows producing engineered primary immune cells within a limited time frame of about 15 to 30 days, preferably between 15 and 20 days, and most preferably between 18 and 20 days so that they keep their full immune therapeutic potential, especially with respect to their cytotoxic activity.

These cells form a population of cells, which preferably originate from a single donor or patient. These populations of cells can be expanded under closed culture recipients to comply with highest manufacturing practices requirements and can be frozen prior to infusion into a patient, thereby providing “off the shelf” or “ready to use” therapeutic compositions.

As per the present invention, a significant number of cells originating from the same Leukapheresis can be obtained, which is critical to obtain sufficient doses for treating a patient. Although variations between populations of cells originating from various donors may be observed, the number of immune cells procured by a leukapheresis is generally about from 10⁸ to 10¹⁰ cells of PBMC. PBMC comprises several types of cells: granulocytes, monocytes and lymphocytes, among which from 30 to 60% of T-cells, which generally represents between 10⁸ to 10⁹ of primary T-cells from one donor. The method of the present invention generally ends up with a population of engineered cells that reaches generally more than about 10⁸ T-cells, more generally more than about 10⁹ T-cells, even more generally more than about 10¹⁰ T-cells, and usually more than 10¹¹ T-cells.

The invention is thus more particularly drawn to a therapeutically effective population of primary immune cells, wherein at least 30%, preferably 50%, more preferably 80% of the cells in said population have been modified according to any one the methods described herein.

According to a preferred aspect of the invention, more than 50% of the immune cells comprised in said population are TCR negative T-cells. According to a more preferred aspect of the invention, more than 50% of the immune cells comprised in said population are CAR positive T-cells. Engineered immune cells, Populations of cells, therapeutic compositions and uses.

The present invention is particularly focused on populations of cells which can comprise mixture of engineered cells comprising one or several attributes, which are obtainable by the methods described herein, for their use as therapeutic compositions.

Such populations of cells generally comprise at least 25%, preferably at least 50%, more preferably at least 75% of cells, having at least one of said genetic modification(s).

Such populations of cells preferably comprise at least 25%, preferably at least 50%, more preferably at least 75% of immune cells having at least two, preferably at least three, preferably at least four, even more preferably at least five of said genetic modification(s) also referred to herein as “attributes”.

Also the invention encompasses therapeutic compositions comprising a population of engineered immune cells characterized by the following (phenotypic) attributes:
Exogenous expression of a CAR targeting a tMUC1 epitope, and
Reduced B2M expression by at least 30%; preferably at least 50%, more preferably at least 75% of the cells; and/or
Reduced PD1 expression by at least 30%; preferably at least 50%, more preferably at least 75%;
and/or
Optionally, reduced TCR expression by at least 50%; preferably by at least 75%;
Optionally, increased IL-12, IL-15 or IL-18 expression by at least 30%; preferably by at least 50%, more preferably by at least 75%;
Optionally reduced TGFβ expression, by at least 30%; preferably 50%, more preferably by at least 75%;
Optionally, exogenous expression of a decoy of TGFβR2,
Optionally secretion of HYAL1, HYAL2 and SPAM1 by introduction of exogenous coding sequences; and
Optionally expression of GP11, PCK1 and/or LDHA by introduction of exogenous coding sequences.

The above percentage of expression can reflect more than 80%, 90%, 95% or 100% of the cells in the population having at least one of these phenotypes. The percentages are based on a comparison with non-engineered cells in the same culture conditions, for instance by measures of overall protein expression.

From the genotype perspective, the populations of cells according to the present invention can be characterized by the following genetic features:
At least 50% of the immune cells displaying an exogenous polynucleotide sequence encoding a CAR targeting a tMUC1 epitope; and
At least 50% of the immune cells display B2M inactive allele(s) preferably at least 75%;
and/or
At least 30% of the immune cells display mutated PD1 allele(s), preferably at least 50%, more preferably 75%;
Optionally, at least 50% of T-cells display TCR inactive allele(s), preferably at least 75%;
Optionally, at least 30% of the immune cells display exogenously introduced sequences encoding IL-12, IL-15 or IL-18, preferably at least 50%, more preferably at least 75%;
Optionally, at least 20% of the immune cells display sequences encoding a decoy of TGFβR2 exogenously inserted in their genome, preferably at least 50%, more preferably at least 75%;
Optionally, at least 20% of the immune cells display sequences encoding HYAL1, HYAL2 and/or SPAM1 exogenously inserted in their genome, preferably at least 50%, more preferably at least 75%;
Optionally at least 20% of the immune cells display sequences encoding GPI1 PCK1 and/or LDHA exogenously inserted in their genome, preferably at least 50%, more preferably at least 75%;
Optionally, at least 30% of the immune cells display mutated TGFβ allele(s), preferably at least 50%, more preferably at least 75%:

The above percentages can reflect more than 80%, 90%, 95% or 100% of the cells in the population comprising at least one of the genetic modifications. The percentages are based on a comparison with non-engineered cells, for instance by quantitative PCR performed on the overall cell population or representative samples thereof.

In such populations, it can be desirable that at least one TCRalpha allele is disrupted in more than 90%, preferably more than 95% of the cells for their subsequent allogeneic use in patients. In preferred embodiments, said TCR alpha gene is disrupted by the insertion of an exogenous sequence coding for a genetic attribute, such as the CAR itself or dnTGFBRII (SEQ ID NO:59) or both. Such decoy of TGFβR2 can be co-expressed with the CAR upon viral vector transduction (rLV or AAV vectors comprising both coding sequences).

According to preferred embodiments, B2M and/or PD1 allele is/are disrupted in said populations of cells by the insertion of an exogenous sequence encoding NK inhibitor, such as HLA-E (SEQ ID NO:60) or HLA-G (SEQ ID NO:61) or any functional sequences having at least 80%, 90% or 95% identity with same.

According to preferred embodiments, B2M and/or PD1 allele is/are disrupted in said populations of cells by the insertion of an exogenous sequence encoding IL-12a (SEQ ID NO:63) and/or IL12b (SEQ ID NO:64), IL-15a (SEQ ID NO:66) or IL-18 (SEQ ID NO:68) or any functional sequences having at least 80%, 90% or 95% identity with same.

According to preferred embodiments, said populations of cells comprise exogenous sequences encoding HYAL1 (SEQ ID NO:69), HYAL2 (SEQ ID NO:70), SPAM1 (SEQ ID NO:71), GP11(SEQ ID NO:72), PCK1(SEQ ID NO:73) or LDHA (SEQ ID NO:74), or any functional sequences having at least 80%, 90% or 95% identity with same, which are preferably inserted at PD1, CD69, CD25 or GMCSF loci.

According to preferred embodiments, said populations of cells comprise cells further mutated to confer resistance to lymphodepletion treatments, such as to inactivate or reduce expression of CD52 and/or dCK gene allele(s).

Said populations of cells preferably, but not necessarily, comprise an exogenous polynucleotide sequence encoding a CAR targeting a tMUC1 epitope, such as CLS MUC1-A, CLS MUC1-B, CLS MUC1-C, CLS MUC1-D. Such exogenous polynucleotide sequence encoding a CAR targeting a tMUC1 epitope has preferably at least 80%, 90% or 95% identity with respectively SEQ ID NO:205, SEQ ID NO:206, SEQ ID NO:207 and SEQ ID NO:208.

Such compositions or populations of cells can therefore be used as medicaments; especially for treating cancer, particularly for the treatment of lymphoma, but also for solid tumors such as melanomas, neuroblastomas, gliomas or carcinomas such as lung, breast, colon, prostate or ovary tumors in a patient in need thereof.

The invention is more particularly drawn to populations of primary TCR negative T-cells originating from a single donor, wherein at least 20%, preferably 30%, more preferably 50% of the cells in said population have been modified using sequence-specific reagents in at least two, preferably three different loci.

In another aspect, the present invention relies on methods for treating patients in need thereof, said method comprising at least one of the following steps:
(a) Determining specific antigen markers present at the surface of patients tumors biopsies;
(b) providing a population of engineered primary immune cells engineered by one of the methods of the present invention as previously described, preferably expressing a recombinant receptor directed against said specific antigen markers;
(c) Administrating said engineered population of engineered primary immune cells to said patient,

Generally, said populations of cells mainly comprises CD4 and CD8 positive immune cells, such as T-cells, which can undergo robust in vivo T cell expansion and can persist for an extended amount of time in-vitro and in-vivo.

The treatments involving the engineered primary immune cells according to the present invention can be ameliorating, curative or prophylactic. It may be either part of an autologous immunotherapy or part of an allogenic immunotherapy treatment.

In another embodiment, said isolated cell according to the invention or cell line derived from said isolated cell can be used for the treatment of solid tumors, in particular solid tumors, such as typically: oesophageal cancer, breast cancer, gastric cancer, cholangiocarcinoma, pancreatic cancer, colon cancer, lung cancer, thymic carcinoma, mesothelioma, ovarian cancer and/or endometrial cancer.

Adult tumors/cancers and pediatric tumors/cancers are also included.

The treatment with the engineered immune cells according to the invention may be in combination with one or more therapies against cancer selected from the group of antibodies therapy, chemotherapy, cytokines therapy, dendritic cell therapy, gene therapy, hormone therapy, laser light therapy and radiation therapy.

According to a preferred embodiment of the invention, said treatment can be administrated into patients undergoing an immunosuppressive treatment. Indeed, the present invention preferably relies on cells or population of cells, which have been made resistant to at least one immunosuppressive agent due to the inactivation of a gene encoding a receptor for such immunosuppressive agent. In this aspect, the immunosuppressive treatment should help the selection and expansion of the T-cells according to the invention within the patient.

The administration of the cells or population of cells according to the present invention may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous or intralymphatic injection, or intraperitoneally. In one embodiment, the cell compositions of the present invention are preferably administered by intravenous injection.

The administration of the cells or population of cells can consist of the administration of 10⁴-10⁹ cells per kg body weight, preferably 10⁵ to 10⁶ cells/kg body weight including all integer values of cell numbers within those ranges. The present invention thus can provide more than 10, generally more than 50, more generally more than 100 and usually more than 1000 doses comprising between 10⁶ to 10⁸ gene edited cells originating from a single donor's or patient's sampling.

The cells or population of cells can be administrated in one or more doses. In another embodiment, said effective amount of cells are administrated as a single dose. In another embodiment, said effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions within the skill of the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, said effective amount of cells or composition comprising those cells are administrated parenterally. Said administration can be an intravenous administration. Said administration can be directly done by injection within a tumor.

In certain embodiments of the present invention, cells are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or nataliziimab treatment for MS patients or efaliztimab treatment for psoriasis patients or other treatments for PML patients. In further embodiments, the T cells of the invention may be used in combination with chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludaribine, cyclosporin, FK506, rapamycin, mycoplienolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin) (Henderson, Naya et al. 1991; Liu, Albers et al. 1992; Bierer, Hollander et al. 1993). In a further embodiment, the cell compositions of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH, In another embodiment, the cell compositions of the present invention are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

The present invention is also particularly drawn to a general method of treating solid tumor(s) in a patient, comprising the steps of immunodepleting said patient with a lymphodepletion regimen and infusing genetically engineered lymphocytes made resistant to the lymphodepletion agent used in the lymphodepletion regimen and specifically targeting said solid tumor(s). Such genetically engineered lymphocytes are preferably CAR positive T-cells, more preferably endowed with a anti-MUC1CAR as described herein.

The lymphodepletion regimen preferably comprises an antibody directed against an antigen present at the surface of immune cells, such as CD52, CD3, CD4, CD8, CD45, or other specific markers, or being drugs such as purine analogues (ex: fludarabine and/or chlorofarabine) and glucocorticoids.

According to a preferred embodiment of the invention, the method comprises submitting the patient to a lymphodepletion regimen comprising an antibody directed against CD52, and administrating an engineered CAR T-cell endowed with an anti-MUC1 CAR, which expression of CD52 is reduced, deficient or inactivated.

As a preferred embodiment of the present invention, the lymphodepleting treatment can comprise an anti-CD52 antibody, such as alemtuzumab, alone or in combination. The lymphodepletion regimen may for instance combine cyclophosphamide, typically for 1 to 3 days, fludarabine for 1 to 5 days, and alemtuzumab from 1 to 5 days. In general, the lymphodepletion regimen can comprise cyclophosphamide between 50 and 70 mg/kg/day, fludarabine between 20 and 40 mg/m2/day, and alemtuzumab 0.1 to 0.5 mg/kg/day alone or in combination.

To this aim, the present invention provides with the combined use of a composition for 5 lymphodepleting a patient affected by a solid tumor, said composition comprising an anti-CD52 antibody, and a population of engineered lymphocytes targeting MUC1 that are not sensitive to said antibody, such population preferably comprising cells that express anti-MUC1 CAR and have impaired CD52 expression. In such engineered cells, allele(s) of the CD52 gene has been preferably inactivated by a rare-cutting endonuclease, such as a TALE-nuclease or a RNA-guided endonuclease as previously described.

The present invention also methods for treating a patient having a condition characterized by MUC1 expressing cells, comprising the steps of:
Engineering immune cells from a donor to express a functional anti-MUC1 CAR as previously described;
Administrating said CAR positive engineered immune cells to a patient to eliminate cells expressing a tMUC1 epitope.

In preferred embodiments, this method comprises a previous treatment step in which the patient is lymphodepleted. In this respect, said CAR positive engineered immune cells can be mutated to confer resistance to said lymphodepletion treatment. For instance, said CAR positive engineered immune cells can be mutated in its CD52 gene to get resistant to anti-CD52 treatment, such as alemtuzumab.

When applied to allogeneic settings, such protocol can be regarded as a method for treating a patient having a condition characterized by MUC1 expressing cells, wherein said method combines the administration of (1) a lymphodepleting agent and (2) a population of allogeneic engineered immune cells from a donor to express a chimeric antigen receptor (CAR) specifically directed against a tMUC1 epitope.

The present invention also provides with a medical kit comprising said lymphodepleting composition and said population of engineered cells resistant thereto for its use in solid tumors cancer treatment.

By “cytolytic activity” or “cytotoxic activity” or “cytotoxicity” is meant the percentage of cell lysis of target cells conferred by an immune cell.

A method for determining the cytotoxicity is described below:

With adherent target cells: 2·10⁴ specific target antigen (STA)-positive or STA-negative cells are seeded in 0.1 ml per well in a 96 well plate. The day after the plating, the STA-positive and the STA-negative cells are labeled with CellTrace CFSE and co-cultured with 4×10⁵ T cells for 4 hours. The cells are then harvested, stained with a fixable viability dye (eBioscience) and analyzed using the MACSQuant flow cytometer (Miltenyi).

With suspension target cells: STA-positive and STA-negative cells are respectively labeled with CellTrace CFSE and CellTrace Violet. About 2×10⁴ ROR1-positive cells are co-cultured with 2×10⁴ STA-negative cells with 4×10⁵ T cells in 0.1 ml per well in a 96-well plate. After a 4 hour incubation, the cells are harvested and stained with a fixable viability dye (eBioscience) and analyzed using the MACSQuant flow cytometer (Miltenyi).

The percentage of specific lysis can be calculated using the following formula:

$$\% cell lysis = 100 \% - \frac{\frac{\begin{matrix}\% viable target cells upon \\ coculture with CAR modified T cells\end{matrix}}{\begin{matrix}\% viable control cells upon \\ coculture with CAR modified T cells\end{matrix}}}{\frac{\begin{matrix}\% viable target cells upon \\ coculture with non modified T cells\end{matrix}}{\begin{matrix}\% viable control cells upon \\ coculture with non modified T cells\end{matrix}}}$$

By “increased cytotoxicity” is meant that the % cell lysis of target cells conferred by the engineered immune cells is increased by at least 10%, such as at least 20%, at least 30%, at least 40%, at least 50%, at least 60%, at least 70%, at least 80%, at least 90% or at least 100% or more, compared to the % cell lysis of target cells conferred by the immune cell not being engineered.
“identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 70%, 85%, 90%, 95%, 98% or 99% identity to specific polypeptides described herein and preferably exhibiting substantially the same functions, as well as polynucleotide encoding such polypeptides, are contemplated.

Unless otherwise stated, the present invention encompasses polypeptides and polynucleotides sharing at least 70%, generally at least 80%, more generally at least 85%, preferably at least 90%, more preferably at least 95% and even more preferably at least 97% with those described herein.
The term “subject” or “patient” as used herein generally refers to mammalians, preferably to primates and more preferably to humans.

The above written description of the invention provides a manner and process of making and using it such that any person skilled in this art is enabled to make and use the same, this enablement being provided in particular for the subject matter of the appended claims, which make up a part of the original description.

Where a numerical limit or range is stated herein, the endpoints are included. Also, all values and subranges within a numerical limit or range are specifically included as if explicitly written out.

Having generally described this invention, a further understanding can be obtained by reference to certain specific examples, which are provided herein for purposes of illustration only, and are not intended to limit the scope of the claimed invention.

### Examples

### Example 1: In-Vitro Experiments and Anti-MUC1 CAR-T Production

### Selection of UCARTMUC1 Target Cell Lines for In Vitro Assays:

Target cell lines T47D (ATCC® HTB-133™) and HCC70 (ATCC® CRL-2315™) were purchased from American Type Culture Collection (ATCC) MUC1 negative control cell line 293FT (R70007) was purchased from ThermoFisher Scientific. All cell lines were cultured according to manufacturer recommendations.

Cell surface expression of tumor associated MUC1 (tMUC1) was determined using flow cytometry. Adherent cells, T47D, HCC70 and 293FT were detached from cell culture flasks using Accutase Cell Dissociation Reagent (Biolegend: 423201) and were subsequently stained with commercial anti MUC1 antibodies HMFG2 (BD Biosciences: 566590) 16A (Biolegend: 355608) or SM3 (Invitrogen: 53989382). Data was acquired on BD FACS CANTO II flow cytometer and analyzed in FlowJo.

Reporter cell lines T47D-NanoLuc-GFP and HCC70-NanoLuc-GFP were generated by transducing wild type T47D and HCC70 cells with rLV bearing the nucleotide sequence encoding NanoLuciferase (NanoLuc) and EGFP separated by a self-cleaving peptide T2A form a single bi-cistronic transcript. MUC1 expression of reporter cell lines was evaluated as described above.

In Vitro Specificity of Top 4 Anti-MUC1 scFVs:

Anti-MUC1 scFVs (CLS MUC1-A, CLS MUC1-B, CLS MUC1-C and CLS MUC1-D) as described in Tables 3 to 6 were produced by Lake Pharma (201 Industrial Road San Carlos, CA 94070) as a recombinant protein fused to mouse FC. Primary cells from tissues known to express high level of normal MUC1: kidney (PCS-400-012), Lung (PCS-300-010) and Cervix (PCS-480-011) were obtained from ATCC and were cultured according to manufacturer recommendations. T47D, HCC70 and 293FT cells were used as positive and negative controls (respectively). All cells were detached using Accutase Cell Dissociation solution and were stained with recombinant scFV-FC protein followed by PE conjugated goat anti-mouse FC gamma specific antibody (Jackson Immunoresearch: 115-115-164) or commercial HMFG2 antibody (BD Biosciences: 566590). Data was acquired on BD FACS CANTO II instrument and analyzed in FlowJo. For comparison cells were also stained with commercial anti-TROP2 antibodies (Biolegend (NY18): 363804 and Miltenyi (REA916): 130-115-055). The FlowJo graph analysis is displayed in FIG. 2, where it is observed that commercial HMFG2 antibody as well as recombinant scFVs (MUC1-A-scFV, MUC1-B-scFV, MUC1-C and MUC1-D-scFV) stain tMUC1 expressing breast cancer cell lines T47D and HCC70 but fail to stain primary cells from tissues expressing normal MUC1 (kidney, lung and cervix) or MUC1 negative control cell line 293FT. These results demonstrate strong specificity of the 4 scFV candidates for tMUC1.

### Production of MUC1 CAR-T Cells:

At day 0, frozen human Peripheral Blood Mononuclear Cells (PBMC) from AllCells (Alameda, California 94502) were thawed, washed, counted and resuspended in X-vivo 15 medium (Lonza: 04-418Q) supplemented with 5% AB serum (GeminiBio: 100-318) and 20 ng/mL recombinant human IL-2 (Miltenyi: 130-097-743). The cells were then transferred to an incubator set at 37° C., 5% CO2.

At day 1, PBMC were counted, analyzed by flow cytometry to assess the % of CD3+ cells, centrifuged and resuspended in X-vivo 15 medium supplemented with 5% AB serum, 20 ng/mL human IL-2 and Dynabeads Human T activator CD3/CD28 (ThermoFisher #11161D: 25 μl per million of CD3+ cells). The cells were then transferred to an incubator set at 37° C., 5% CO2.

At day 4, T cells and rLV vectors bearing the polynucleotide sequences encoding the anti MUC1 CARs were resuspended in X-vivo 15 medium supplemented with 5% AB serum and 20 ng/ml IL-2, and seeded over retronectin coated plates. The plates were then transferred to an incubator set at 37° C., 5% CO2.

At day 5, T cells were sub-cultured into fresh X-vivo 15 medium-supplemented with 5% AB serum, 20 ng/ml IL-2. The cells were then transferred to an incubator set at 37° C., 5% CO2.

At day 7, T-cells were sub-cultured into fresh X-vivo 15 medium-supplemented with 5% AB serum, 20 ng/ml IL-2. If the CAR-T cells were engineered with additional attributes (TCR and PD1 knockout, and inducible IL-12 release), cells were passaged in the morning at 1e6 cells/mL in X-vivo 15 medium-supplemented with 5% AB serum, 20 ng/ml IL-2. Six hours later, cells were co-electroporated with mRNA encoding the right and the left arms of respectively the TRAC TALEN (SEQ ID NO:75 and 76) and PD1 TALEN (SEQ ID NO:77 and 78) as previously reported [Poirot et al. (2013) Blood. 122 (21): 1661 and Sachdeva et al. (2019) Nat Commun. 10 (1)] to efficiently inactivate TCRα and PD-1 genes and prevent TCRαβ expression at the surface of the primary T-cells. TALEN® is the registered name for the heterodimeric TALE-nucleases designed by Cellectis (8, rue de la Croix Jarry, 75013 Paris, France) that use Fok1 as nuclease catalytic domain as initially described by Voytas et al. in WO2011072246.

Transfection was performed using the AgilePulse technology. Cells were placed in an incubator set at 37° C., 5% CO2 for 15 minutes, following this step cells were pelleted and resuspended with 200 uL of X-vivo 15 medium-supplemented with 5% AB serum, 20 ng/ml IL-2. Cells were transduced with AAV6, bearing polynucleotide sequence encoding IL-12 cytokine and LNGFR reporter gene separated by a self-cleaving peptide T2A, at 50,000 genomes/cell.

At day 8, T cells were transferred to GRex devices for expansion. Between days 8 and 18, T cells were expanded in GRex devices. When GRex10 or GRex 6 multi-well cell culture plates were used, 75% of the culture media was removed at day 13 and replaced with fresh medium containing IL-2, and fresh IL-2 was added at day 11 and 15. During the expansion period, the cell cultures were incubated at 37° C. under 5% CO2.

At day 18, the totality of the UCART cells was cryopreserved for later use in in vitro and in vivo assays.

In order to engineer next-generation MUC-1 CAR T-cells [dnTGFBR2]^{pos} [TCR]^{neg} [B2M]^{neg} [HLA-E]^{pos} [PD1]^{neg} [IL-12/LNGFR]^{pos}, (FIG. 5) the following protocol was used:

At day 0, frozen human Peripheral Blood Mononuclear Cells (PBMC) from AllCells (Alameda, California 94502) were thawed, washed, counted and resuspended in X-vivo 15 medium (Lonza: 04-418Q) supplemented with 5% AB serum (GeminiBio: 100-318) and 20 ng/mL recombinant human IL-2 (Miltenyi: 130-097-743). The cells were then transferred to an incubator set at 37° C., 5% CO2.

At day 1, PBMC were counted, analyzed by flow cytometry to assess the % of CD3+ cells, centrifuged and resuspended in X-vivo 15 medium supplemented with 5% AB serum, 20 ng/mL human IL-2 and Dynabeads Human T activator CD3 CD28 (ThermoFisher #11161D: 25 μl per million of CD3+ cells). The cells were then transferred to an incubator set at 37° C., 5% CO2.

At day 3, T cells and rLV vectors bearing the polynucleotide sequences encoding the anti-MUC1 CARs expressed from a biscistronic construct with dnTGFBR2 were resuspended in X-vivo 15 medium supplemented with 5% AB serum and 20 ng/ml IL-2 and seeded over retronectin coated plates. The plates were then transferred to an incubator set at 37° C., 5% CO2.

At day 5, T cells were co-electroporated with mRNA encoding the right and the left arms of respectively the TRAC TALEN (SEQ ID NO:75 and 76) and B2M TALEN (SEQ ID NO:79 and 80) and transduced 30 minutes later with HLA-E AAV6 (SEQ ID NO:85) following the protocol previously reported [Poirot et al. (2013) Blood. 122 (21): 1661 and Sachdeva et al. (2019) Nat Commun. 10 (1)] to efficiently inactivate TCRα and B2M gene and prevent TCRαp expression at the surface of the primary T-cells. TALEN® is the registered name for the heterodimeric TALE-nucleases designed by Cellectis (8, rue de la Croix Jarry, 75013 Paris, France) that use Fok1 as nuclease catalytic domain as initially described by Voytas et al. in WO2011072246.

At day 6, engineered T cells were sub-cultured into fresh X-vivo 15 medium-supplemented with 5% AB serum and 20 ng/ml IL-2.

At day 7, T-cells were electroporated with mRNA encoding the right and the left arms of respectively the PD-1 TALEN (SEQ ID NO:77 and 78) following the protocol previously reported [Poirot et al. (2013) Blood. 122 (21): 1661 and Sachdeva et al. (2019) Nat Commun. 10 (1)] and transduced 30 minutes later with IL-12-LNGFR AAV6 (SEQ ID NO:84) as previously reported.

Transfection was performed using the AgilePulse technology. Cells were placed in an incubator set at 37° C., 5% CO2 for 15 minutes, following this step cells were pelleted and resuspended with 200 uL of X-vivo 15 medium-supplemented with 5% AB serum, 20 ng/ml IL-2. Cells were transduced with AAV6, bearing polynucleotide sequence encoding HLA-E on day 5 or IL-12 cytokine and LNGFR reporter gene separated by a self-cleaving peptide T2A on day 7, at 50,000 genomes/cell. Following transduction cells were transferred to an incubator set at 30° C., 5% CO2, and incubated overnight.

At day 8, T cells were transferred to GRex devices for expansion. Between days 8 and 18, T cells were expanded in GRex devices. When GRex10 or GRex 6 multi-well cell culture plates were used, 75% of the culture media was removed at day 13 and replaced with fresh medium containing IL-2, and fresh IL-2 was added at day 11 and 15. During the expansion period, the cell cultures were incubated at 37° C. under 5% CO2.

At day 18, the totality of the UCART cells was cryopreserved for later use in in vitro and in vivo assays.

In order to engineer next-generation MUC-1 CAR T-cells [TGFBR2]^{neg} [TCR]^{neg} [B2M]^{neg}[HLA-E]^{pos} [PD1]^{neg}[IL12/LNGFR]^{pos}, (FIG. 17) the following protocol was used:

At day 0, frozen human Peripheral Blood Mononuclear Cells (PBMC) from AllCells (Alameda, California 94502) were thawed, washed, counted and resuspended in X-vivo 15 medium (Lonza: 04-418Q) supplemented with 5% AB serum (GeminiBio: 100-318) and 20 ng/mL recombinant human IL-2 (Miltenyi: 130-097-743). The cells were then transferred to an incubator set at 37° C., 5% CO2.

At day 1, PBMC were counted, analyzed by flow cytometry to assess the % of CD3+ cells, centrifuged and resuspended in X-vivo 15 medium supplemented with 5% AB serum, 20 ng/mL human IL-2 and Dynabeads Human T activator CD3/CD28 (ThermoFisher #11161D: 25 μl per million of CD3+ cells). The cells were then transferred to an incubator set at 37° C., 5% CO2.

At day 2, T cells and rLV vectors bearing the polynucleotide sequences encoding the anti MUC1 CARs were resuspended in X-vivo 15 medium supplemented with 5% AB serum and 20 ng/ml IL-2, and seeded over retronectin coated plates. The plates were then transferred to an incubator set at 37° C., 5% CO2.

At day 3, T-cells were sub-cultured into fresh X-vivo 15 medium-supplemented with 5% AB serum, 20 ng/ml IL-2. If the CAR-T cells were engineered with additional attributes (TCR and B2M knockout, and HLA-E), cells were passaged in the morning at 1e6 cells/mL in X-vivo 15 medium-supplemented with 5% AB serum, 20 ng/ml IL-2. Six hours later, cells were co-electroporated with mRNA encoding the right and the left arms of respectively the TRAC TALEN (SEQ ID NO:75 and 76) and B2M TALEN (SEQ ID NO:79 and 80) as previously reported [Poirot et al. (2013) Blood. 122 (21): 1661 and Sachdeva et al. (2019) Nat Commun. 10 (1)] to efficiently inactivate TCRα and B2M genes and prevent TCRαβ expression at the surface of the primary T-cells. TALEN is the registered name for the heterodimeric TALE-nucleases designed by Cellectis (8, rue de la Croix Jarry, 75013 Paris, France) that use Fok1 as nuclease catalytic domain as initially described by Voytas et al. in WO2011072246.

Transfection was performed using the AgilePulse technology. Cells were placed in an incubator set at 37° C., 5% CO2 for 15 minutes, following this step cells were pelleted and resuspended with 200 uL of X-vivo 15 medium-supplemented with 5% AB serum, 20 ng/ml IL-2. Cells were transduced with AAV6, bearing polynucleotide sequence encoding HLA-E gene separated by a self-cleaving peptide T2A, at 50,000 genomes/cell.

At day 7, T cells were further engineered with additional attributes (TGFBR2 and PD1 KO and IL12 expression). T-cells were passed at 1e6 cells/mL in X-vivo 15 medium-supplemented with 5% AB serum, 20 ng/ml IL-2. Six hours later, cells were co-electroporated with mRNA encoding the left and right arms of the PD1 TALEN (SEQ ID NO:77 and 78) and TGFBR2 TALEN (SEQ ID NO: 223 and 224) as previously reported to efficiently inactivate TGFBR2 and PD1 genes and to allow specific IL-12 expression upon MUC1 recognition and T-cell activation.

At day 9, T cells were transferred to GRex devices for expansion. Between days 9 and 18, T cells were expanded in GRex devices at 37° C. under 5% CO2with medium exchange from time to time.

At day 18, the totality of the UCART cells was cryopreserved for later use in in vitro and in vivo assays.

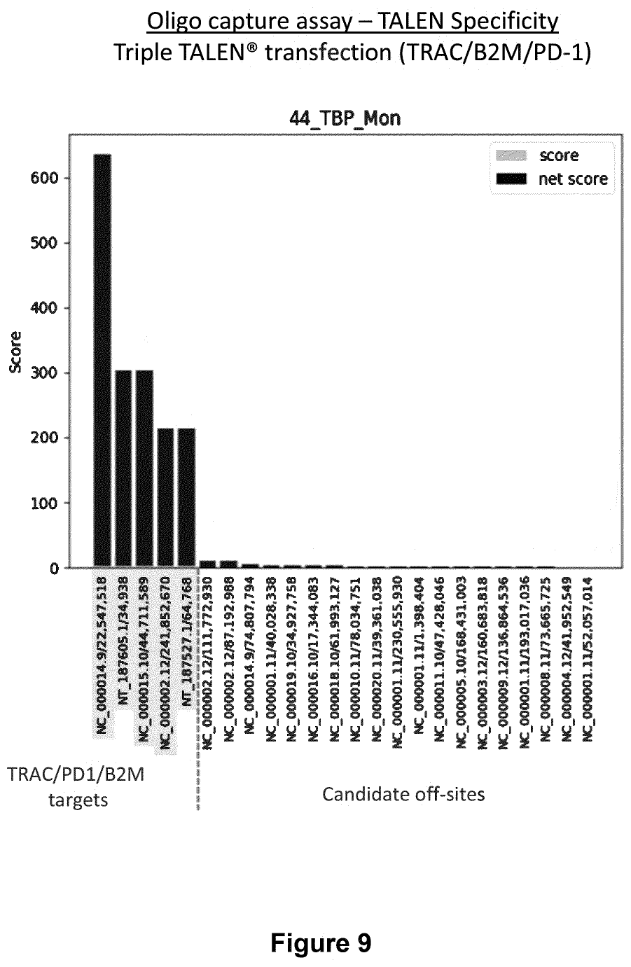

To analyze genomic on-target and potential off-target sites arising from co-transfection of several TALEN simultaneously, we used an oligo captured assay coupled with next-generation sequencing adapted from previous assay (Tsai et al. 2015). Only on-targets presented a high score, suggesting that no off-target could be detected with the combination of TRAC, B2M and PD1 TALEN that were used to generate UCART MUC1-A [dnTGFBR2]^{pos} [TCR]^{neg} [B2M]^{neg} [HLA-E]^{pos} [PD1]^{neg} [IL-12/LNGFR]^{pos} cells (FIG. 9).

TABLE 14
polynucleotide and polypeptide sequences used
to introduce the genetic attributes into UCART MUC1

| SEQ ID NO: # | Polynucleotide/polypeptide sequences |
|---|---|
| TRAC TALEN Left | MGDPKKKRKVIDIADLRTLGYSQQQQEKIKPKVRSTVAQHHEALVGHGFTHAHIVAL |
| (SEQ ID NO: 75) | SQHPAALGTVAVKYQDMIAALPEATHEAIVGVGKQWSGARALEALLTVAGELRGPP |
| | LQLDTGQLLKIAKRGGVTAVEAVHAWRNALTGAPLNLTPQQVVAIASNGGGKQALE |
| | TVQRLLPVLCQAHGLTPQQVVAIASNNGGKQALETVQRLLPVLCQAHGLTPQQVVA |
| | IASNGGGKQALETVQRLLPVLCQAHGLTPEQVVAIASHDGGKQALETVQRLLPVLCQ |
| | AHGLTPEQVVAIASHDGGKQALETVQRLLPVLCQAHGLTPEQVVAIASHDGGKQAL |
| | ETVQRLLPVLCQAHGLTPEQVVAIASNIGGKQALETVQALLPVLCQAHGLTPEQVVAI |
| | ASHDGGKQALETVQRLLPVLCQAHGLTPEQVVAIASNIGGKQALETVQALLPVLCQA |
| | HGLTPQQVVAIASNNGGKQALETVQRLLPVLCQAHGLTPEQVVAIASNIGGKQALET |
| | VQALLPVLCQAHGLTPQQVVAIASNGGGKQALETVQRLLPVLCQAHGLTPEQVVAI |
| | ASNIGGKQALETVQALLPVLCQAHGLTPQQVVAIASNGGGKQALETVQRLLPVLCQ |
| | AHGLTPEQVVAIASHDGGKQALETVQRLLPVLCQAHGLTPQQVVAIASNGGGRPAL |
| | ESIVAQLSRPDPALAALTNDHLVALACLGGRPALDAVKKGLGDPISRSQLVKSELEEKK |
| | SELRHKLKYVPHEYIELIEIARNSTQDRILEMKVMEFFMKVYGYRGKHLGGSRKPDGAI |
| | YTVGSPIDYGVIVDTKAYSGGYNLPIGQADEMQRYVEENQTRNKHINPNEWWKVYP |
| | SSVTEFKFLFVSGHFKGNYKAQLTRLNHITNCNGAVLSVEELLIGGEMIKAGTLTLEEV |
| | RRKFNNGEINFAAD |
| TRAC TALEN Right | MGDPKKKRKVIDIADLRTLGYSQQQQEKIKPKVRSTVAQHHEALVGHGFTHAHIVAL |
| (SEQ ID NO: 76) | SQHPAALGTVAVKYQDMIAALPEATHEAIVGVGKQWSGARALEALLTVAGELRGPP |
| | LQLDTGQLLKIAKRGGVTAVEAVHAWRNALTGAPLNLTPEQVVAIASHDGGKQALE |
| | TVQRLLPVLCQAHGLTPQQVVAIASNGGGKQALETVQRLLPVLCQAHGLTPEQVVAI |
| | ASHDGGKQALETVQRLLPVLCQAHGLTPEQVVAIASNIGGKQALETVQALLPVLCQA |
| | HGLTPQQVVAIASNNGGKQALETVQRLLPVLCQAHGLTPEQVVAIASHDGGKQALE |
| | TVQRLLPVLCQAHGLTPQQVVAIASNGGGKQALETVQRLLPVLCQAHGLTPQQVVA |
| | IASNNGGKQALETVQRLLPVLCQAHGLTPQQVVAIASNNGGKQALETVQRLLPVLC |
| | QAHGLTPQQVVAIASNGGGKQALETVQRLLPVLCQAHGLTPEQVVAIASNIGGKQA |
| | LETVQALLPVLCQAHGLTPEQVVAIASHDGGKQALETVQRLLPVLCQAHGLTPEQVV |
| | AIASNIGGKQALETVQALLPVLCQAHGLTPEQVVAIASHDGGKQALETVQRLLPVLC |
| | QAHGLTPQQVVAIASNNGGKQALETVQRLLPVLCQAHGLTPQQVVAIASNGGGRP |
| | ALESIVAQLSRPDPALAALTNDHLVALACLGGRPALDAVKKGLGDPISRSQLVKSELEE |
| | KKSELRHKLKYVPHEYIELIEIARNSTQDRILEMKVMEFFMKVYGYRGKHLGGSRKPD |
| | GAIYTVGSPIDYGVIVDTKAYSGGYNLPIGQADEMQRYVEENQTRNKHINPNEWWK |
| | VYPSSVTEFKFLFVSGHFKGNYKAQLTRLNHITNCNGAVLSVEELLIGGEMIKAGTLTL |
| | EEVRRKFNNGEINFAAD |
| PD1 TALEN Left | MGDPKKKRKVIDIADLRTLGYSQQQQEKIKPKVRSTVAQHHEALVGHGFTHAHIVAL |
| (SEQ ID NO: 77) | SQHPAALGTVAVKYQDMIAALPEATHEAIVGVGKQWSGARALEALLTVAGELRGPP |
| | LQLDTGQLLKIAKRGGVTAVEAVHAWRNALTGAPLNLTPEQVVAIASKLGGKQALET |
| | VQALLPVLCQAHGLTPEQVVAIASHDGGKQALETVQRLLPVLCQAHGLTPEQVVAIA |
| | SHDGGKQALETVQRLLPVLCQAHGLTPQQVVAIASNGGGKQALETVQRLLPVLCQA |
| | HGLTPEQVVAIASHDGGKQALETVQRLLPVLCQAHGLTPQQVVAIASNGGGKQALE |
| | TVQRLLPVLCQAHGLTPQQVVAIASYKGGKQALETVQRLLPVLCQAHGLTPQQVVAI |
| | ASNGGGKQALETVQRLLPVLCQAHGLTPQQVVAIASNNGGKQALETVQRLLPVLCQ |
| | AHGLTPQQVVAIASNNGGKQALETVQRLLPVLCQAHGLTPQQVVAIASNNGGKQA |
| | LETVQRLLPVLCQAHGLTPQQVVAIASNNGGKQALETVQRLLPVLCQAHGLTPEQV |
| | VAIASHDGGKQALETVQRLLPVLCQAHGLTPEQVVAIASHDGGKQALETVQRLLPVL |
| | CQAHGLTPEQVVAIASNIGGKQALETVQALLPVLCQAHGLTPQQVVAIASNGGGRP |
| | ALESIVAQLSRPDPALAALTNDHLVALACLGGRPALDAVKKGLGDPISRSQLVKSELEE |
| | KKSELRHKLKYVPHEYIELIEIARNSTQDRILEMKVMEFFMKVYGYRGKHLGGSRKPD |
| | GAIYTVGSPIDYGVIVDTKAYSGGYNLPIGQADEMQRYVEENQTRNKHINPNEWWK |
| | VYPSSVTEFKFLFVSGHFKGNYKAQLTRLNHITNCNGAVLSVEELLIGGEMIKAGTLTL |
| | EEVRRKFNNGEINFAAD |
| PD1 TALEN Right | MGDPKKKRKVIDIADLRTLGYSQQQQEKIKPKVRSTVAQHHEALVGHGFTHAHIVAL |
| (SEQ ID NO: 78) | SQHPAALGTVAVKYQDMIAALPEATHEAIVGVGKQWSGARALEALLTVAGELRGPP |
| | LQLDTGQLLKIAKRGGVTAVEAVHAWRNALTGAPLNLTPEQVVAIASHDGGKQALE |
| | TVQRLLPVLCQAHGLTPQQVVAIASNGGGKQALETVQRLLPVLCQAHGLTPEQVVAI |
| | ASHDGGKQALETVQRLLPVLCQAHGLTPQQVVAIASNGGGKQALETVQRLLPVLCQ |
| | AHGLTPQQVVAIASNGGGKQALETVQRLLPVLCQAHGLTPQQVVAIASNGGGKQA |
| | LETVQRLLPVLCQAHGLTPQQVVAIASNNGGKQALETVQRLLPVLCQAHGLTPEQV |
| | VAIASNIGGKQALETVQALLPVLCQAHGLTPQQVVAIASNGGGKQALETVQRLLPVL |
| | CQAHGLTPEQVVAIASHDGGKQALETVQRLLPVLCQAHGLTPQQVVAIASNGGGK |
| | QALETVQRLLPVLCQAHGLTPQQVVAIASNNGGKQALETVQRLLPVLCQAHGLTPE |
| | QVVAIASNGGKQALETVQRLLPVLCQAHGLTPQQVVAIASNNGGKQALETVQRLLP |
| | VLCQAHGLTPEQVVAIASHDGGKQALETVQRLLPVLCQAHGLTPQQVVAIASNGGG |
| | RPALESIVAQLSRPDPALAALTNDHLVALACLGGRPALDAVKKGLGDPISRSQLVKSEL |
| | EEKKSELRHKLKYVPHEYIELIEIARNSTQDRILEMKVMEFFMKVYGYRGKHLGGSRKP |
| | DGAIYTVGSPIDYGVIVDTKAYSGGYNLPIGQADEMQRYVEENQTRNKHINPNEWW |
| | KVYPSSVTEFKFLFVSGHFKGNYKAQLTRLNHITNCNGAVLSVEELLIGGEMIKAGTLT |
| | LEEVRRKFNNGEINFAAD |
| B2M TALEN Left | MGDPKKKRKVIDIADLRTLGYSQQQQEKIKPKVRSTVAQHHEALVGHGFTHAHIVAL |
| (SEQ ID NO: 79) | SQHPAALGTVAVKYQDMIAALPEATHEAIVGVGKQWSGARALEALLTVAGELRGPP |
| | LQLDTGQLLKIAKRGGVTAVEAVHAWRNALTGAPLNLTPQQVVAIASNGGGKQALE |
| | TVQRLLPVLCQAHGLTPEQVVAIASNIGGKQALETVQALLPVLCQAHGLTPQQVVAI |
| | ASNNGGKQALETVQRLLPVLCQAHGLTPEQVVAIASHDGGKQALETVQRLLPVLCQ |
| | AHGLTPQQVVAIASNGGGKQALETVQRLLPVLCQAHGLTPQQVVAIASNNGGKQA |
| | LETVQRLLPVLCQAHGLTPQQVVAIASNGGGKQALETVQRLLPVLCQAHGLTPQQV |
| | VAIASNNGGKQALETVQRLLPVLCQAHGLTPEQVVAIASHDGGKQALETVQRLLPVL |
| | CQAHGLTPQQVVAIASNGGGKQALETVQRLLPVLCQAHGLTPEQVVAIASHDGGK |
| | QALETVQRLLPVLCQAHGLTPQQVVAIASNNGGKQALETVQRLLPVLCQAHGLTPE |
| | QVVAIASHDGGKQALETVQRLLPVLCQAHGLTPQQVVAIASNNGGKQALETVQRLL |
| | PVLCQAHGLTPEQVVAIASHDGGKQALETVQRLLPVLCQAHGLTPQQVVAIASNGG |
| | GRPALESIVAQLSRPDPALAALTNDHLVALACLGGRPALDAVKKGLGDPISRSQLVKS |
| | ELEEKKSELRHKLKYVPHEYIELIEIARNSTQDRILEMKVMEFFMKVYGYRGKHLGGSR |
| | KPDGAIYTVGSPIDYGVIVDTKAYSGGYNLPIGQADEMQRYVEENQTRNKHINPNE |
| | WWKVYPSSVTEFKFLFVSGHFKGNYKAQLTRLNHITNCNGAVLSVEELLIGGEMIKA |
| | GTLTLEEVRRKFNNGEINFAAD |
| B2M TALEN Right | MGDPKKKRKVIDIADLRTLGYSQQQQEKIKPKVRSTVAQHHEALVGHGFTHAHIVAL |
| (SEQ ID NO: 80) | SQHPAALGTVAVKYQDMIAALPEATHEAIVGVGKQWSGARALEALLTVAGELRGPP |
| | LQLDTGQLLKIAKRGGVTAVEAVHAWRNALTGAPLNLTPQQVVAIASNNGGKQALE |
| | TVQRLLPVLCQAHGLTPQQVVAIASNNGGKQALETVQRLLPVLCQAHGLTPEQVVAI |
| | ASNIGGKQALETVQALLPVLCQAHGLTPQQVVAIASNGGGKQALETVQRLLPVLCQ |
| | AHGLTPEQVVAIASNIGGKQALETVQALLPVLCQAHGLTPQQVVAIASNNGGKQAL |
| | ETVQRLLPVLCQAHGLTPEQVVAIASHDGGKQALETVQRLLPVLCQAHGLTPEQVVA |
| | IASHDGGKQALETVQRLLPVLCQAHGLTPQQVVAIASNGGGKQALETVQRLLPVLC |
| | QAHGLTPEQVVAIASHDGGKQALETVQRLLPVLCQAHGLTPEQVVAIASHDGGKQA |
| | LETVQRLLPVLCQAHGLTPEQVVAIASNIGGKQALETVQALLPVLCQAHGLTPQQVV |
| | AIASNNGGKQALETVQRLLPVLCQAHGLTPQQVVAIASNNGGKQALETVQRLLPVLC |
| | QAHGLTPEQVVAIASHDGGKQALETVQRLLPVLCQAHGLTPQQVVAIASNGGGRPA |
| | LESIVAQLSRPDPALAALTNDHLVALACLGGRPALDAVKKGLGDPISRSQLVKSELEEK |
| | KSELRHKLKYVPHEYIELIEIARNSTQDRILEMKVMEFFMKVYGYRGKHLGGSRKPDG |
| | AIYTVGSPIDYGVIVDTKAYSGGYNLPIGQADEMQRYVEENQTRNKHINPNEWWKV |
| | YPSSVTEFKFLFVSGHFKGNYKAQLTRLNHITNCNGAVLSVEELLIGGEMIKAGTLTLE |
| | EVRRKFNNGEINFAAD |
| TGFBR2 TALEN | MGDPKKKRKVIDIADLRTLGYSQQQQEKIKPKVRSTVAQHHEALVGHGFTHAHIVAL |
| Left | SQHPAALGTVAVKYQDMIAALPEATHEAIVGVGKQWSGARALEALLTVAGELRGPP |
| (SEQ ID NO: 223) | LQLDTGQLLKIAKRGGVTAVEAVHAWRNALTGAPLNLTPDQVVAIASHDGGKQALE |
| | TVQRLLPVLCQDHGLTPDQVVAIASHDGGKQALETVQRLLPVLCQDHGLTPDQVVA |
| | IASHDGGKQALETVQRLLPVLCQDHGLTPDQVVAIASNGGGKQALETVQRLLPVLCQ |
| | DHGLTPDQVVAIASNIGGKQALETVQALLPVLCQDHGLTPDQVVAIASNGGGKQAL |
| | ETVQRLLPVLCQDHGLTPDQVVAIASNNGGKQALETVQRLLPVLCQDHGLTPDQVV |
| | AIASNIGGKQALETVQALLPVLCQDHGLTPDQVVAIASNNGGKQALETVQRLLPVLC |
| | QDHGLTPDQVVAIASNNGGKQALETVQRLLPVLCQDHGLTPDQVVAIASNIGGKQA |
| | LETVQALLPVLCQDHGLTPDQVVAIASNNGGKQALETVQRLLPVLCQDHGLTPDQV |
| | VAIASNGGGKQALETVQRLLPVLCQDHGLTPDQVVAIASNIGGKQALETVQALLPVL |
| | CQDHGLTPDQVVAIASNGGGKQALETVQRLLPVLCQDHGLTPQQVVAIASNGGGR |
| | PALESIVAQLSRPDPALAALTNDHLVALACLGGRPALDAVRRGLGDPISRSQLVKSELE |
| | EKKSELRHKLKYVPHEYIELIEIARNSTQDRILEMKVMEFFMKVYGYRGKHLGGSRKP |
| | DGAIYTVGSPIDYGVIVDTKAYSGGYNLPIGQADEMQRYVEENQTRNKHINPNEWW |
| | KVYPSSVTEFKFLFVSGHFKGNYKAQLTRLNHITNCNGAVLSVEELLIGGEMIKAGTLT |
| | LEEVRRKFNNGEINFAAD |
| TGFBR2 TALEN | MGDPKKKRKVIDIADLRTLGYSQQQQEKIKPKVRSTVAQHHEALVGHGFTHAHIVAL |
| Right | SQHPAALGTVAVKYQDMIAALPEATHEAIVGVGKQWSGARALEALLTVAGELRGPP |
| (SEQ ID NO: 224) | LQLDTGQLLKIAKRGGVTAVEAVHAWRNALTGAPLNLTPDQVVAIASNNGGKQALE |
| | TVQRLLPVLCQDHGLTPDQVVAIASNIGGKQALETVQALLPVLCQDHGLTPDQVVAI |
| | ASNNGGKQALETVQRLLPVLCQDHGLTPDQVVAIASNIGGKQALETVQALLPVLCQ |
| | DHGLTPDQVVAIASNIGGKQALETVQALLPVLCQDHGLTPDQVVAIASNNGGKQAL |
| | ETVQRLLPVLCQDHGLTPDQVVAIASNIGGKQALETVQALLPVLCQDHGLTPDQVVA |
| | IASNGGGKQALETVQRLLPVLCQDHGLTPDQVVAIASNNGGKQALETVQRLLPVLC |
| | QDHGLTPDQVVAIASNGGGKQALETVQRLLPVLCQDHGLTPDQVVAIASHDGGKQ |
| | ALETVQRLLPVLCQDHGLTPDQVVAIASHDGGKQALETVQRLLPVLCQDHGLTPDQ |
| | VVAIASNGGGKQALETVQRLLPVLCQDHGLTPDQVVAIASNGGGKQALETVQRLLP |
| | VLCQDHGLTPDQVVAIASHDGGKQALETVQRLLPVLCQDHGLTPQQVVAIASNGG |
| | GRPALESIVAQLSRPDPALAALTNDHLVALACLGGRPALDAVRRGLGDPISRSQLVKS |
| | ELEEKKSELRHKLKYVPHEYIELIEIARNSTQDRILEMKVMEFFMKVYGYRGKHLGGSR |
| | KPDGAIYTVGSPIDYGVIVDTKAYSGGYNLPIGQADEMQRYVEENQTRNKHINPNE |
| | WWKVYPSSVTEFKFLFVSGHFKGNYKAQLTRLNHITNCNGAVLSVEELLIGGEMIKA |
| | GTLTLEEVRRKFNNGEINFAAD |
| TRAC target | TGATCCTCTTGTCCCACAGATATCCAGAACCCTGACCCTGCCGTGTACCAGCTGAG |
| sequence | AGA |
| SEQ ID NO: 225 | |
| PD1 target | TACCTCTGTGGGGCCATCTCCCTGGCCCCCAAGGCGCAGATCAAAGAGA |
| sequence | |
| SEQ ID NO: 226 | |
| B2M target | TTAGCTGTGCTCGCGCTACTCTCTCTTTCTGGCCTGGAGGCTATCCA |
| sequence | |
| SEQ ID NO: 227 | |
| TGFBR2 target | TCCCTATGAGGAGTATGCCTCTTGGAAGACAGAGAAGGACATCTTCTCA |
| sequence | |
| SEQ ID NO: 87 | |
| AAV6 PD1KO IL- | GACTCCCCAGACAGGCCCTGGAACCCCCCCACCTTCTCCCCAGCCCTGCTCGTGG |
| 12 | TGACCGAAGGGGACAACGCCACCTTCACCTGCAGCTTCTCCAACACATCGGAGA |
| SEQ ID NO: 84 | GCTTCGTGCTAAACTGGTACCGCATGAGCCCCAGCAACCAGACGGACAAGCTGG |
| | CCGCCTTCCCCGAGGACCGCAGCCAGCCCGGCCAGGACTGCCGCTTCCGTGTCA |
| | CACAACTGCCCAACGGGCGTGACTTCCACATGAGCGTGGTCAGGGCCCGGCGC |
| | AATGACAGCGGCACCTACCTCTGTGGGGCCGGTTCTGGCGTGAAACAGACTTTG |
| | AATTTTGACCTTCTCAAGTTGGCGGGAGACGTGGAGTCCAACCCAGGGCCCATG |
| | TGGCCCCCTGGGTCAGCCTCCCAGCCACCGCCCTCACCTGCCGCGGCCACAGGT |
| | CTGCATCCAGCGGCTCGCCCTGTGTCCCTGCAGTGCCGGCTCAGCATGTGTCCA |
| | GCGCGCAGCCTCCTCCTTGTGGCTACCCTGGTCCTCCTGGACCACCTCAGTTTGG |
| | CCAGAAACCTCCCCGTGGCCACTCCAGACCCAGGAATGTTCCCATGCCTTCACCA |
| | CTCCCAAAACCTGCTGAGGGCCGTCAGCAACATGCTCCAGAAGGCCAGACAAAC |
| | TCTAGAATTTTACCCTTGCACTTCTGAAGAGATTGATCATGAAGATATCACAAAA |
| | GATAAAACCAGCACAGTGGAGGCCTGTTTACCATTGGAATTAACCAAGAATGAG |
| | AGTTGCCTAAATTCCAGAGAGACCTCTTTCATAACTAATGGGAGTTGCCTGGCCT |
| | CCAGAAAGACCTCTTTTATGATGGCCCTGTGCCTTAGTAGTATTTATGAAGACTT |
| | GAAGATGTACCAGGTGGAGTTCAAGACCATGAATGCAAAGCTTCTGATGGATCC |
| | TAAGAGGCAGATCTTTCTAGATCAAAACATGCTGGCAGTTATTGATGAGCTGAT |
| | GCAGGCCCTGAATTTCAACAGTGAGACTGTGCCACAAAAATCCTCCCTTGAAGA |
| | ACCGGATTTTTATAAAACTAAAATCAAGCTCTGCATACTTCTTCATGCTTTCAGAA |
| | TTCGGGCAGTGACTATTGATAGAGTGATGAGCTATCTGAATGCTTCCGGAAGCG |
| | GAGCTACTAACTTCAGCCTGCTGAAGCAGGCTGGAGACGTGGAGGAGAACCCT |
| | GGACCTATGTGTCACCAGCAGTTGGTCATCTCTTGGTTTTCCCTGGTTTTTCTGGC |
| | ATCTCCCCTCGTGGCCATATGGGAACTGAAGAAAGATGTTTATGTCGTAGAATT |
| | GGATTGGTATCCGGATGCCCCTGGAGAAATGGTGGTCCTCACCTGTGACACCCC |
| | TGAAGAAGATGGTATCACCTGGACCTTGGACCAGAGCAGTGAGGTCTTAGGCTC |
| | TGGCAAAACCCTGACCATCCAAGTCAAAGAGTTTGGAGATGCTGGCCAGTACAC |
| | CTGTCACAAAGGAGGCGAGGTTCTAAGCCATTCGCTCCTGCTGCTTCACAAAAA |
| | GGAAGATGGAATTTGGTCCACTGATATTTTAAAGGACCAGAAAGAACCCAAAAA |
| | TAAGACCTTTCTAAGATGCGAGGCCAAGAATTATTCTGGACGTTTCACCTGCTGG |
| | TGGCTGACGACAATCAGTACTGATTTGACATTCAGTGTCAAAAGCAGCAGAGGC |
| | TCTTCTGACCCCCAAGGGGTGACGTGCGGAGCTGCTACACTCTCTGCAGAGAGA |
| | GTCAGAGGGGACAACAAGGAGTATGAGTACTCAGTGGAGTGCCAGGAGGACA |
| | GTGCCTGCCCAGCTGCTGAGGAGAGTCTGCCCATTGAGGTCATGGTGGATGCCG |
| | TTCACAAGCTCAAGTATGAAAACTACACCAGCAGCTTCTTCATCAGGGACATCAT |
| | CAAACCTGACCCACCCAAGAACTTGCAGCTGAAGCCATTAAAGAATTCTCGGCA |
| | GGTGGAGGTCAGCTGGGAGTACCCTGACACCTGGAGTACTCCACATTCCTACTT |
| | CTCCCTGACATTCTGCGTTCAGGTCCAGGGCAAGAGCAAGAGAGAAAAGAAAG |
| | ATAGAGTCTTCACGGACAAGACCTCAGCCACGGTCATCTGCCGCAAAAATGCCA |
| | GCATTAGCGTGCGGGCCCAGGACCGCTACTATAGCTCATCTTGGAGCGAATGGG |
| | CATCTGTGCCCTGCAGTGAGGGCAGAGGCAGCCTGCTGACCTGCGGCGACGTC |
| | GAGGAGAACCCCGGGCCCATGGGGGCAGGTGCCACCGGCCGCGCCATGGACG |
| | GGCCGCGCCTGCTGCTGTTGCTGCTTCTGGGGGTGTCCCTTGGAGGTGCCAAGG |
| | AGGCATGCCCCACAGGCCTGTACACACACAGCGGTGAGTGCTGCAAAGCCTGCA |
| | ACCTGGGCGAGGGTGTGGCCCAGCCTTGTGGAGCCAACCAGACCGTGTGTGAG |
| | CCCTGCCTGGACAGCGTGACGTTCTCCGACGTGGTGAGCGCGACCGAGCCGTGC |
| | AAGCCGTGCACCGAGTGCGTGGGGCTCCAGAGCATGTCGGCGCCGTGCGTGGA |
| | GGCCGATGACGCCGTGTGCCGCTGCGCCTACGGCTACTACCAGGATGAGACGA |
| | CTGGGCGCTGCGAGGCGTGCCGCGTGTGCGAGGCGGGCTCGGGCCTCGTGTTC |
| | TCCTGCCAGGACAAGCAGAACACCGTGTGCGAGGAGTGCCCCGACGGCACGTA |
| | TTCCGACGAGGCCAACCACGTGGACCCGTGCCTGCCCTGCACCGTGTGCGAGGA |
| | CACCGAGCGCCAGCTCCGCGAGTGCACACGCTGGGCCGACGCCGAGTGCGAGG |
| | AGATCCCTGGCCGTTGGATTACACGGTCCACACCCCCAGAGGGCTCGGACAGCA |
| | CAGCCCCCAGCACCCAGGAGCCTGAGGCACCTCCAGAACAAGACCTCATAGCCA |
| | GCACGGTGGCAGGTGTGGTGACCACAGTGATGGGCAGCTCCCAGCCCGTGGTG |
| | ACCCGAGGCACCACCGACAACCTCATCCCTGTCTATTGCTCCATCCTGGCTGCTG |
| | TGGTTGTGGGTCTTGTGGCCTACATAGCCTTCAAGAGGTGATCTAGAGGGCCCG |
| | TTTAAACCCGCTGATCAGCCTCGACTGTGCCTTCTAGTTGCCAGCCATCTGTTGTT |
| | TGCCCCTCCCCCGTGCCTTCCTTGACCCTGGAAGGTGCCACTCCCACTGTCCTTTC |
| | CTAATAAAATGAGGAAATTGCATCGCATTGTCTGAGTAGGTGTCATTCTATTCTG |
| | GGGGGGGGGTGGGGCAGGACAGCAAGGGGGAGGATTGGGAAGACAATAGC |
| | AGGCATGCTGGGGATGCGGTGGGCTCTATGACTAGTGGCGAATTCGGCGCAGA |
| | TCAAAGAGAGCCTGCGGGCAGAGCTCAGGGTGACAGGTGCGGCCTCGGAGGC |
| | CCCGGGGCAGGGGTGAGCTGAGCCGGTCCTGGGGTGGGTGTCCCCTCCTGCAC |
| | AGGATCAGGAGCTCCAGGGTCGTAGGGCAGGGACCCCCCAGCTCCAGTCCAGG |
| | GCTCTGTCCTGCACCTGGGGAATGGTGACCGGCATCTCTGTCCTCTAGCTCTGGA |
| | AGCACCCCAGCCCCTCTAGTCTGCCCTCACCCCTGACCCTGACCCTCCACCCTGAC |
| | CCCGTCCTAACCCCTGACCTTTG |
| AAV6 B2MKO | CGCGCACCCCAGATCGGAGGGCGCCGATGTACAGACAGCAAACTCACCCAGTCT |
| HLA-E | AGTGCATGCCTTCTTAAACATCACGAGACTCTAAGAAAAGGAAACTGAAAACGG |
| SEQ ID NO: 85 | GAAAGTCCCTCTCTCTAACCTGGCACTGCGTCGCTGGCTTGGAGACAGGTGACG |
| | GTCCCTGCGGGCCTTGTCCTGATTGGCTGGGCACGCGTTTAATATAAGTGGAGG |
| | CGTCGCGCTGGCGGGCATTCCTGAAGCTGACAGCATTCGGGCCGAGATGTCTCG |
| | CTCCGTGGCCTTAGCTGTGCTCGCGCTACTCTCTCTTAGCGGCCTCGAAGCTGTT |
| | ATGGCTCCGCGGACTTTAATTTTAGGTGGTGGCGGATCCGGTGGTGGCGGTTCT |
| | GGTGGTGGCGGCTCCATCCAGCGTACGCCCAAAATTCAAGTCTACAGCCGACAT |
| | CCTGCAGAGAACGGCAAATCTAATTTCCTGAACTGCTATGTATCAGGCTTTCACC |
| | CTAGCGATATAGAAGTGGACCTGCTGAAAAACGGAGAGAGGATAGAAAAGGTC |
| | GAACACAGCGACCTCTCCTTTTCCAAGGACTGGAGCTTTTATCTTCTGTATTATAC |
| | TGAATTTACACCCACGGAAAAAGATGAGTATGCGTGCCGAGTAAACCACGTCAC |
| | GCTGTCACAGCCCAAAATAGTAAAATGGGATCGCGACATGGGTGGTGGCGGTT |
| | CTGGTGGTGGCGGTAGTGGCGGCGGAGGAAGCGGTGGTGGCGGTTCCGGATC |
| | TCACTCCTTGAAGTATTTCCACACTTCCGTGTCCCGGCCCGGCCGCGGGGAGCCC |
| | CGCTTCATCTCTGTGGGCTACGTGGACGACACCCAGTTCGTGCGCTTCGACAAC |
| | GACGCCGCGAGTCCGAGGATGGTGCCGCGGGCGCCGTGGATGGAGCAGGAGG |
| | GGTCAGAGTATTGGGACCGGGAGACACGGAGCGCCAGGGACACCGCACAGATT |
| | TTCCGAGTGAACCTGCGGACGCTGCGCGGCTACTACAATCAGAGCGAGGCCGG |
| | GTCTCACACCCTGCAGTGGATGCATGGCTGCGAGCTGGGGCCCGACAGGCGCTT |
| | CCTCCGCGGGTATGAACAGTTCGCCTACGACGGCAAGGATTATCTCACCCTGAA |
| | TGAGGACCTGCGCTCCTGGACCGCGGTGGACACGGCGGCTCAGATCTCCGAGC |
| | AAAAGTCAAATGATGCCTCTGAGGCGGAGCACCAGAGAGCCTACCTGGAAGAC |
| | ACATGCGTGGAGTGGCTCCACAAATACCTGGAGAAGGGGAAGGAGACGCTGCT |
| | TCACCTGGAGCCCCCAAAGACACACGTGACTCACCACCCCATCTCTGACCATGAG |
| | GCCACCCTGAGGTGCTGGGCTCTGGGCTTCTACCCTGCGGAGATCACACTGACC |
| | TGGCAGCAGGATGGGGAGGGCCATACCCAGGACACGGAGCTCGTGGAGACCA |
| | GGCCTGCAGGGGATGGAACCTTCCAGAAGTGGGCAGCTGTGGTGGTGCCTTCT |
| | GGAGAGGAGCAGAGATACACGTGCCATGTGCAGCATGAGGGGCTACCCGAGCC |
| | CGTCACCCTGAGATGGAAGCCGGCTTCCCAGCCCACCATCCCCATCGTGGGCAT |
| | CATTGCTGGCCTGGTTCTCCTTGGATCTGTGGTCTCTGGAGCTGTGGTTGCTGCT |
| | GTGATATGGAGGAAGAAGAGCTCAGGTGGAAAAGGAGGGAGCTACTATAAGG |
| | CTGAGTGGAGCGACAGTGCCCAGGGGTCTGAGTCTCACAGCTTGTAACTGTGCC |
| | TTCTAGTTGCCAGCCATCTGTTGTTTGCCCCTCCCCCGTGCCTTCCTTGACCCTGG |
| | AAGGTGCCACTCCCACTGTCCTTTCCTAATAAAATGAGGAAATTGCATCGCATTG |
| | TCTGAGTAGGTGTCATTCTATTCTGGGGGGTGGGGTGGGGCAGGACAGCAAGG |
| | GGGAGGATTGGGAAGACAATAGCAGGCATGCTGGGGATGCGGTGGGCTCTAT |
| | GTCTCTTTCTGGCCTGGAGGCTATCCAGCGTGAGTCTCTCCTACCCTCCCGCTCTG |
| | GTCCTTCCTCTCCCGCTCTGCACCCTCTGTGGCCCTCGCTGTGCTCTCTCGCTCCG |
| | TGACTTCCCTTCTCCAAGTTCTCCTTGGTGGCCCGCCGTGGGGCTAGTCCAGGGC |
| | TGGATCTCGGGGAAGCGGCGGGGTGGCCTGGGAGTGGGGAAGGGGGTGCGC |
| | ACCCGGGACGCGCGCTACTTGCCCCTTTCGGCGGGGAGCAGGGGAGACCTTTG |
| | GCCTACGGCGACGGGAGGGTCGGGACAAAG |

### Detection of CAR Expression:

CLS MUC1-A, CLS MUC1-B, CLS MUC1-C, and CLS MUC1-D different UCART cell products were then evaluated in vitro. CAR surface expression was evaluated by flow cytometry using either Biotin-SP conjugated goat anti-mouse F(ab)2 fragment specific antibody, that detects the mouse F(ab)2 fragment of the CAR constructs or the Biotin-SP conjugated goat anti-human F(ab)2 fragment specific antibody, that detects the human F(ab)2 fragment of the CAR ScFvs or the Alexa Fluor-488 conjugated rituximab that recognizes the R2 suicide switch portion of all the CARs.

### Killing Activity Assays

MUC1 UCART cells endowed CLS MUC1-A, CLS MUC1-B, CLS MUC1-C, and CLS MUC1-D CARs were produced and tested for CAR expression as described above. At day0 target cells, either T47D-NanoLuc-GFP or HCC70-NanoLuc-GFP, were plated in a flat-bottom 96-well plate at a density of 10,000 target cells per well. Cells were plated in their respective complete medium. At day1, medium was removed from target cells and CLS MUC1-A, CLS MUC1-B, CLS MUC1-C, and CLS MUC1-D or the Non-Transduced (NTD) UCART cells were added to target cells at a 5:1, 2.5:1 or 1:1 CAR+ effector to target (E:T) ratio in X-Vivo medium supplemented with 5% AB serum. Target cells were co-cultured with CARs or the NTD control for 48 hours in an incubator set at 37° C., 5% CO2. Subsequently, NanoLuciferase signal was developed. Following removal of medium, wells were washed once with 100 uL of PBS and then incubated with 0.026% Triton X-100 in PBS for 2 minutes with vortexing. A total of 10 uL of lysate was diluted in 40 uL of PBS, mixed with 50 uL of Nano-Glo substrate and incubated for 3 minutes. Following incubation luminescence was read. Percent specific lysate was calculated using the following equation: % Lysis=(1−(Target+scFV UCART)/(Target+NTD UCART))*100.

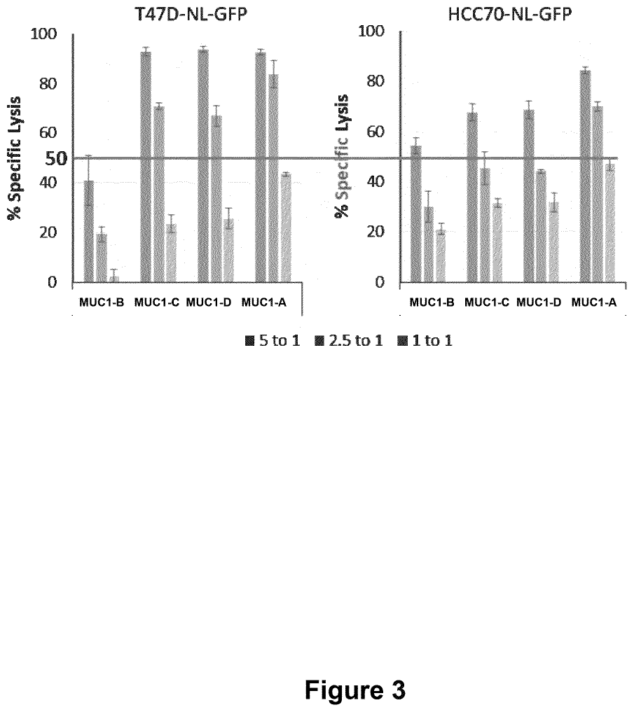

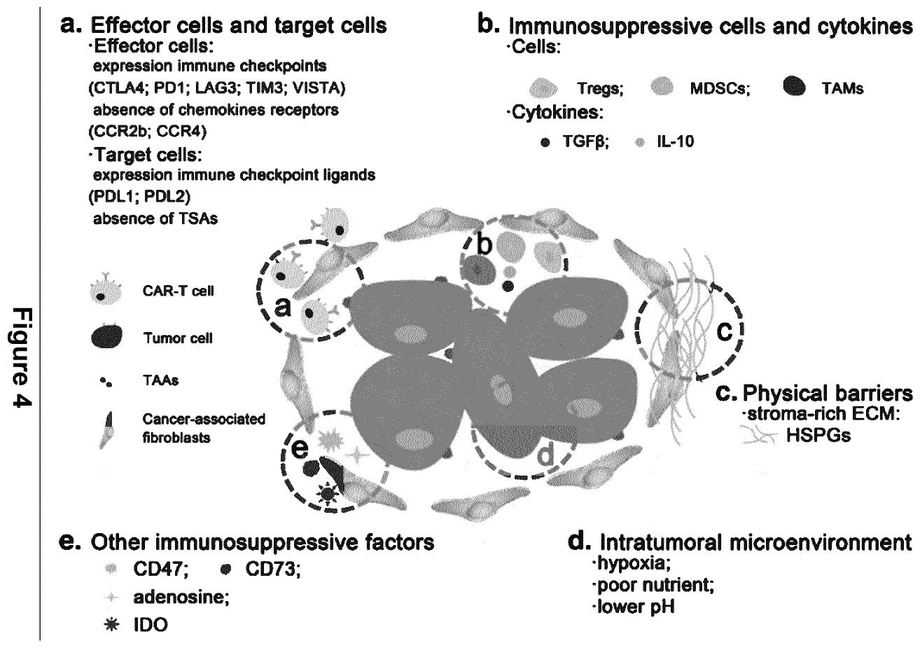

The results are presented in the diagrams of FIG. 3, displaying the % lysis for each of the four anti-MUC1 CAR-T at the different ratios 5:1, 2.5:1 or 1:1. The diagrams show killing activity of the primary T-cells respectively endowed with that the four CAR constructs in a concentration dependent manner. The killing activity induced by the CLS MUC1-A CAR construct was significantly higher than with the other constructs.

### Tumor Microarrays for Detection of MUC1 Expression in Breast Cancer Tumors.

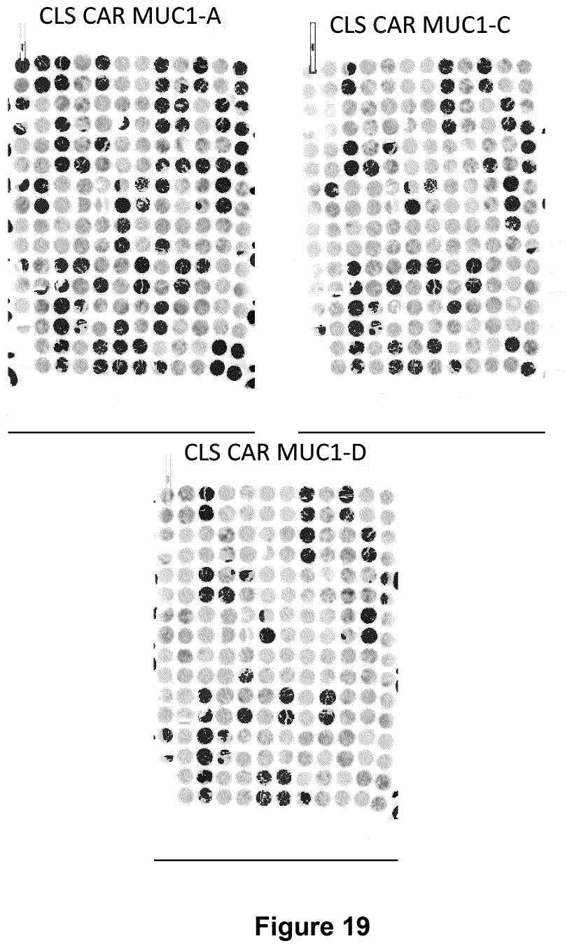

Proteins expressing CLS MUC1-A, CLS MUC1-C and CLS MUC1-D scFVs coupled to the CD8 hinge and mouse IgG1 Fc were produced in CHO cells and purified using protein A. Human paraffin embedded tissue microarrays (TMA) including 84 breast cancer samples were purchased from US Biomax. scFV proteins were used for immunohistochemistry assays. TMAs were dewaxed in consecutive baths: xylene, 100% ethanol, 96% ethanol, 70% ethanol and water. Slides were then washed in reaction buffer and stained in the Discovery XT2 instrument for automated staining. Slides were first incubated with reaction buffer for 32 minutes, then incubated with primary antibodies at 3 ug/ml for CAR CLS MUC1-A and CLS MUC1-C, and 9 ug/ml for CAR MUC1-D for 60 minutes at 37C. Then anti-mouse HRP was applied for 16 minutes and sections were stained with hematoxylin and bluing reagent. Slides were then washed with soap water and tap water and finally mounted and analyzed under the microscope. Specific positive staining was graded as follows: grade 1: minimal; grade 2: slight; grade 3: moderate; grade 4: marked, grade 5: strong. Slides were digitized using a whole scanning device AT2 from Leica (FIG. 19).

### Example 2: In Vivo Anti-MUC1 CAR Activity

### Rationale for the Choice of the Animal Model

Because of the human specificity of the MUC-1 CAR+ T-cells, studies in standard immunocompetent animal models are not applicable due to the rapid targeting and elimination of human T-cells by xenogeneic immune reactions. The animal model selected is the highly immunodeficient NSG mice strain (NOD·Cg-Prkdcscid II2rgtm1WjI/SzJ strain from the Jackson laboratory) as it allows the engraftment of both human MUC1+ tumor cells and human CAR T-cells.

### Validation of the UCART MUC1 Cells Genetic Attributes and TCR Depletion

Less than 20% of the UCART cells produced according to the protocol described in example 1 and modified with CLS MUC1-A CAR remained TCRαβ+, suggesting that the level of TALEN-mediated inactivation of the TCRα gene was highly efficient in the population of cells.

Remaining TCRαβ+ cells were depleted on day 18 of CAR-T cells production. UCART MUC1 cells were counted, pelleted and resuspended in PBS containing 0.5% heat inactivated FBS at a density of 10e8 cells/mL. Subsequently, cells were incubated with anti-TCRα/β-biotin antibody (Miltenyi CliniMACS TCRα/P Kit: 200-070-407) for 30 minutes at 4C at a concentration of 1.875 uL of antibody per every 10e7 cells. Following incubation, cells were washed with 5 volumes of PBS containing 0.5% FBS, resuspended at 10e8 cells/mL and incubated with 3.75 uL of anti-biotin beads per every 10e7 cells (Miltenyi CliniMACS TCRα/P Kit: 200-070-407) for 30 minutes at 4C. Following incubation, cells were washed as before and resuspended at 1.25e8 cells/mL and passed through pre-equilibrated LS column. Flow through containing TCRαβ− cells was collected along with five 1 mL washes. Depletion of TCRαβ+ cells was analyzed by flow cytometry by staining cells with anti-TCRαβ-PE-Vio770 antibody (Miltenyi: 130-119-617). Efficiency of inactivation of PD-1 gene and the efficiency of IL-12 release were tested by direct comparison of cell surface expression of PD-1 and LNGFR reporter on CAR-T cells activated with PMA/ionomycin (40 ng/mL PMA and 2 nM ionomycin) for 24 hours. Following activation, cell surface expression of PD-1 and LNGFR were tested by flow cytometry by staining cells with anti PD-1 or anti LNGFR antibodies. Efficiency of editing for UCART MUC1 modified with CLS MUC1-A and attributes was calculated to be 50% PD-1 knockout and 4.7% LNGFR+. Similar values were achieved for NTD control cells. UCART MUC1 cells modified with CLS MUC1-A CAR without attributes expressed high level of PD-1 (68%) and did not express LNGFR.

### HCC70 Human Tumor Cells Engraftment in NSG Mice

The NSG mice were purchased at 6 weeks old and shaved one week prior to implantation. Wild type HCC70 (ATCC® CRL-2315™) cells were cultured according to the supplier recommendation. On the day on implantation, 80-90% confluent monolayer of HCC70 cells was washed with PBS and then incubated with Accutase Cell Dissociation Reagent in an incubator set at 37° C., 5% CO2. Accutase digestion was quenched by adding 1 volume equivalent of complete growth medium. Cells were pelleted at 300 g for 5 minutes and resuspended with 30 mL of PBS and counted using Vi-cell. Counted cell were pelleted in 50 mL conical tube at 300 g for 5 minutes at 4° C. Pelleted cells were incubated on ice for 5 minutes and then resuspended in 1:1 mixture of matri gel (Corning: 356237) and PBS at a density of 100e6 cell/mL or at 50e6 cells/mL. On day 0, 100 uL of HCC70 cell suspension was implanted into mammary fat pad of NSG mice for a total of 10e6 or 5e6 HCC70 cells per mouse. Tumor volume was measured once a week for 19 days. Good engraftment of HCC70 was observed.

### Tumor Implantation, CAR-T Treatment and Tumor Monitoring

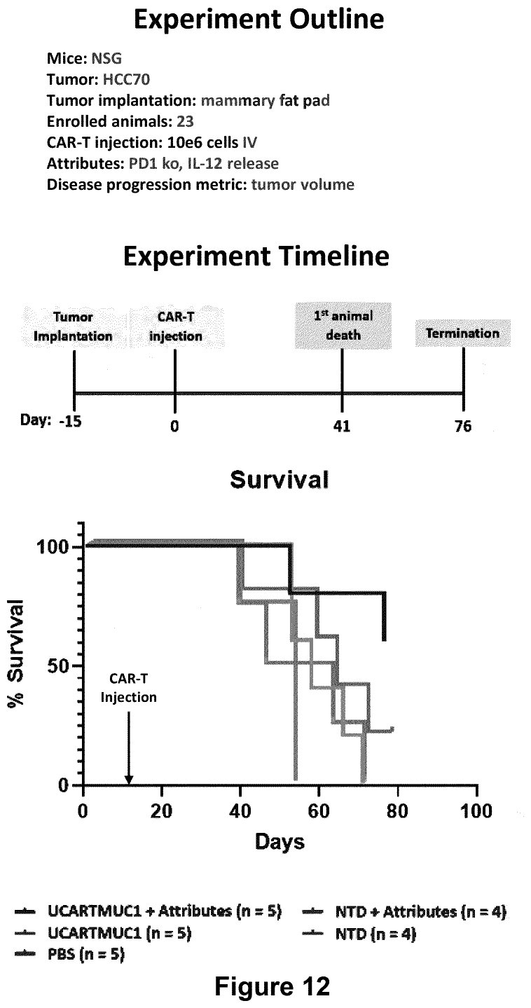

As summarized in FIG. 12, 100 μL of HCC70-NanoLuc-GFP cells at a density of 100e6 cells/mL were injected into the mammary fat pad of the NSG strain of 6 weeks of age. On day 0, mice were infused respectively with 10e6 CAR+UCART CLS MUC1-A and non-transduced corresponding T-cells (NDT) controls. Animals were assigned to each treatment group so that the average tumor volume per group was most similar. Tumor volume was measured once a week. Animals were sacrificed once tumor volume exceeded 2000 mm³ or tumors became ulcerated.

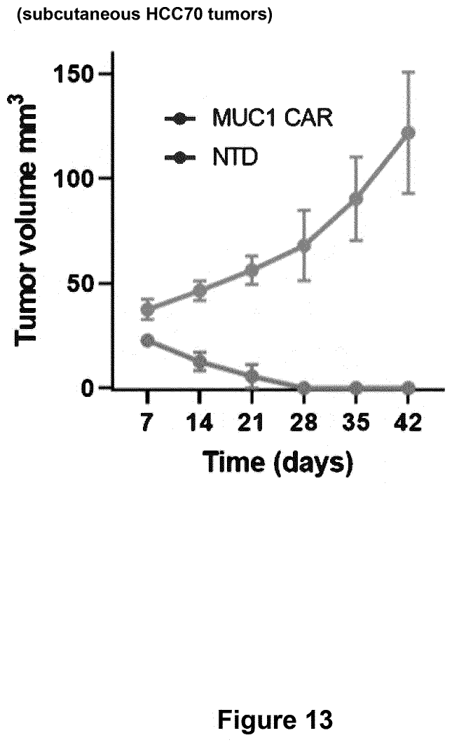

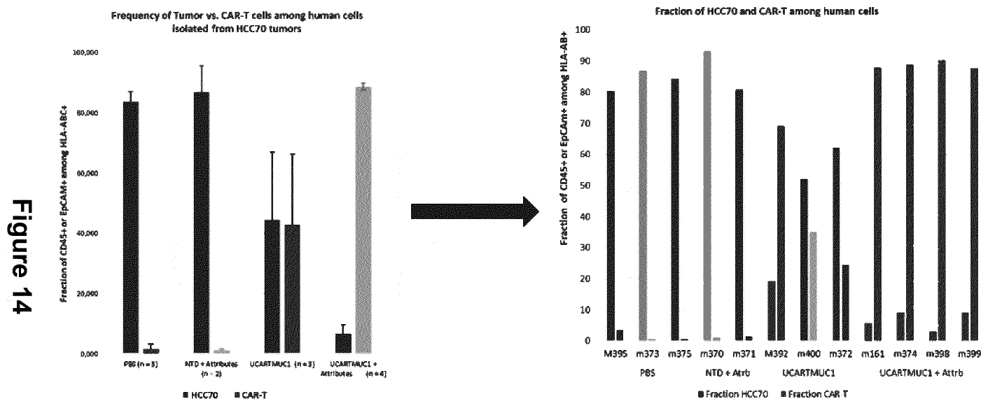

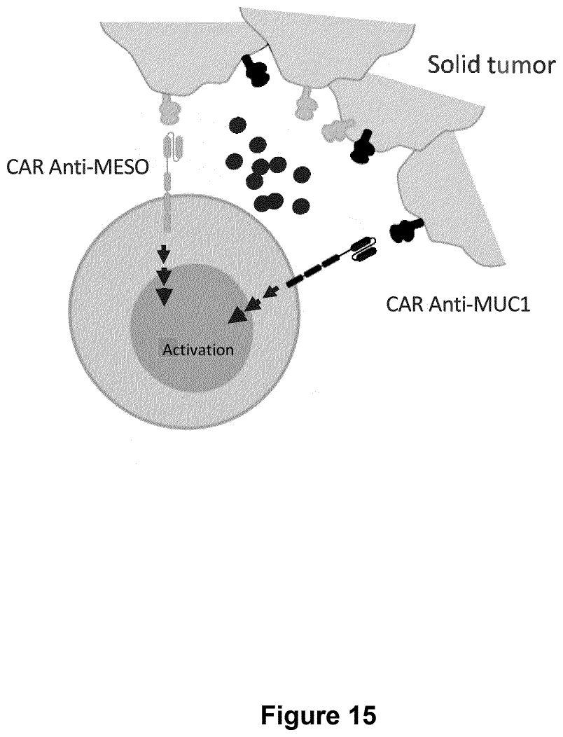

FIGS. 11 and 13 show the results of experiments where the treatment started 7 days post mammary fat implantation or post subcutaneous tumor implantation respectively and where the tumor volume was followed for the indicated days. It can be observed that mice treated with T-cells endowed with CLS MUC1-A CAR were free of tumors by about 28 days, whereas controls had an exponential volume increase of the same tumors.

### CAR-T Cell Engineered for In Vivo Infusion:

UCART cells were engineered and transduced with CLS MUC1-A CAR and comparison were performed with or without attributes (CAR-T CLS MUC1-A or UCART CLS MUC1-A+Attributes). Attributes consisted of TCR knockout, PD-1 knockout and IL-12 release. Similarly, non-transduced control (NTD: CAR negative control T-cells) were produced with or without attributes (NTD and NDT+Attributes). CAR-T cells were produced as described above and were infused fresh from production, without prior freezing. Slight modification to the production was implemented. At day 18 of CAR-T cell production cells were taken out of G-rex and were resuspended at 2e6 cells/mL and were seeded in regular tissue culture flasks. On day 20 of production cells were washed once with PBS, counted and concentrated to 100e6 CAR+ cells/mL. A total of 100 uL of UCART product or controls were injected intravenously.

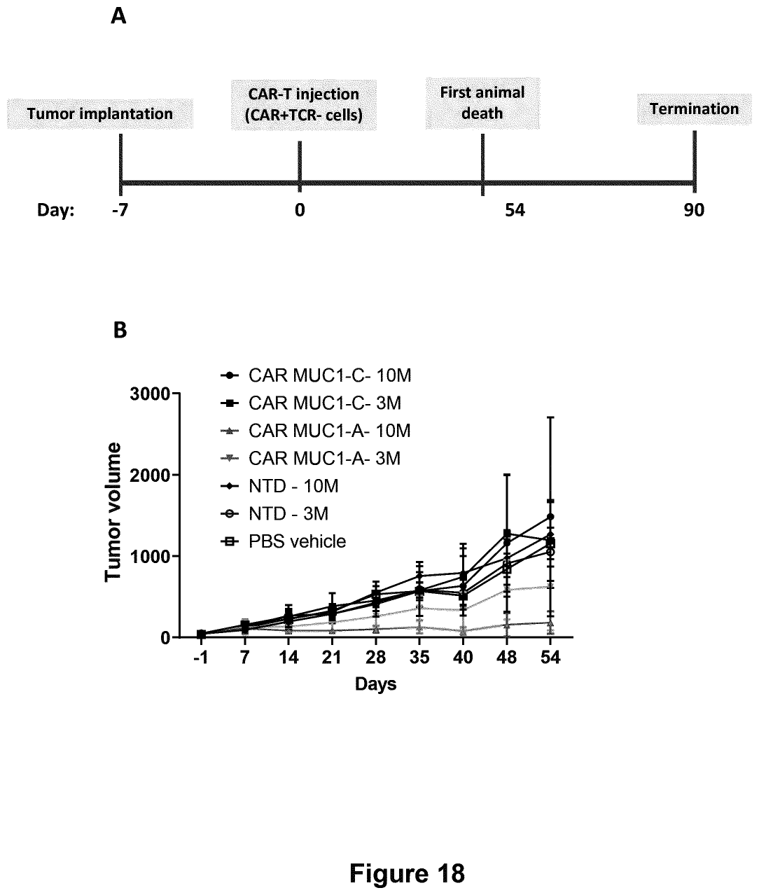

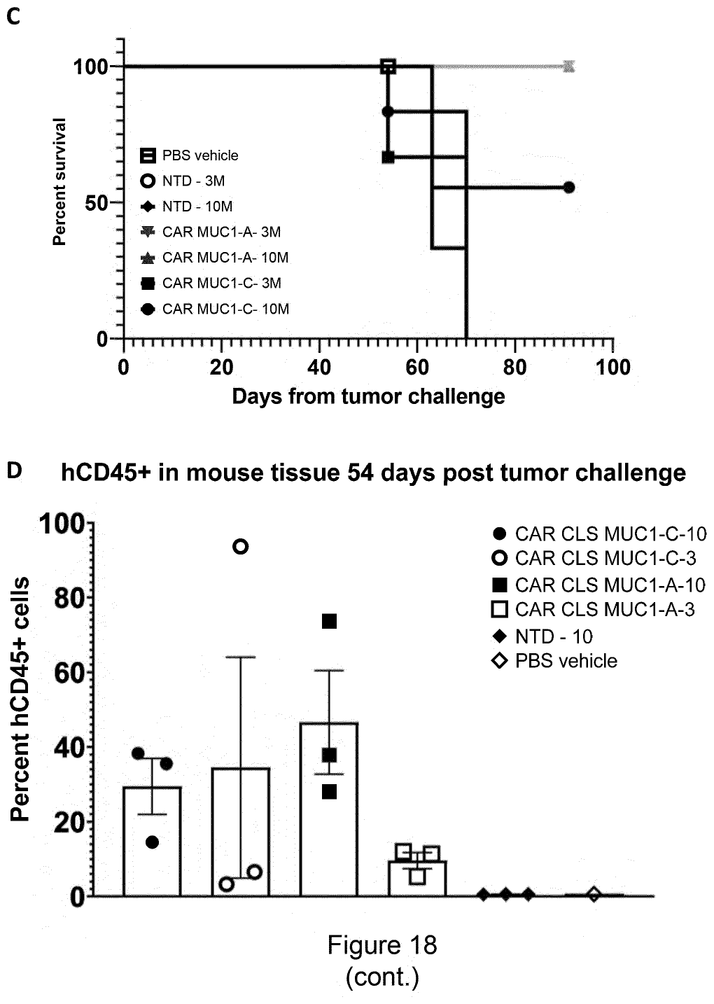

UCART cells were transduced with CLS MUC1-A or CLS MUC-1 C and further engineered for TCR KO, PD1-KO and IL-12 integration. CAR-T cells were produced as described above. Engineered T-cells were infused fresh from production either with 10e6 or 3e6 CAR+UCARTMUC1-A or UCARTMUC1-C, or with 3e6 or 10e6 total non-transduced T-cells (NTD) controls (from the same donor) or PBS. Tumor volume was measured once a week after treatment with CLS MUC1-A or CLS MUC1-C engineered CAR-T cells. Animals were sacrificed once tumor volume exceeded 2000 mm3 or tumors became ulcerated (FIG. 18A). The results demonstrates that UCART bearing CLS MUC1-A could prevent tumor growth at 3 million injected cells and even further at 10 million cells and provided the best survival (FIGS. 18B and C).

FACS Analysis of Tumor Samples Treated with UCARTMUC1+/− Attributes

Tumors were isolated from mice treated as previously described with CAR-T cells transduced with CLS MUC1-A, with or without attributes (PD-1 and TCR knockout and IL-12 release) or with NTD cells (with or without attributes). Tumors were homogenized in Accutase Cell Dissociation Reagent and were transferred to an incubator set at 37° C., 5% CO2 for 30 minutes. Dissociated cells were washed with 20 mL of ice-cold PBS containing 3% FBS and passed through 100 um mesh. Tumor isolates were counted on vi-cell and stained for FACS analysis.

In order to examine CAR-T cell infiltration in tumors as well as the expression of MUC1 on tumor cells, to determine if there is loss of MUC1 expression, which would indicate antigen escape, cells were stained with fixable viability dye e780, to gate on viable cells, with anti-human HLA-ABC-VioBlue and anti-mouse MHCII-PE, to gate on human cells, and with anti-human CD45 and anti-human EpCAM to gate on epithelial tumor cells, and finally with anti-MUC1 (HMFG2 or 16A) antibodies or their corresponding isotype controls to determine MUC1 expression on tumor isolated from individual mice.

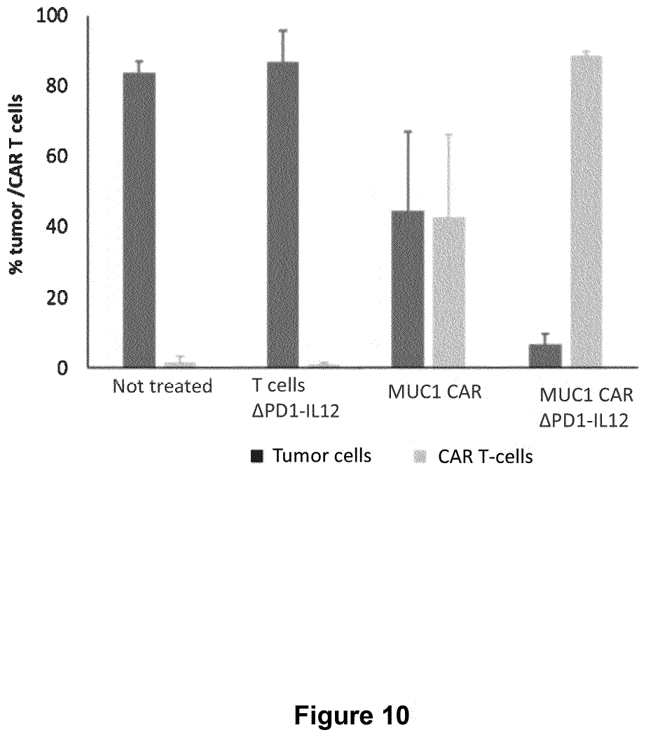

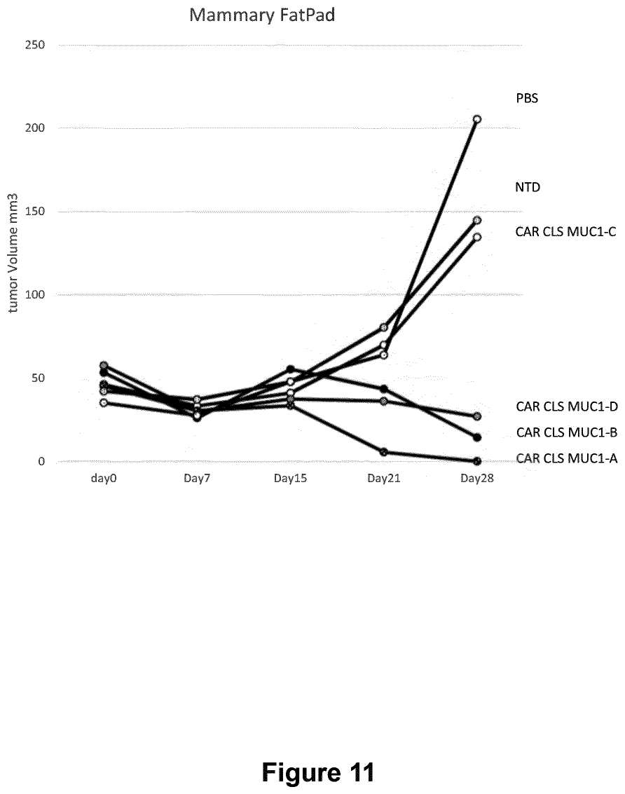

As represented in FIGS. 10 and 14, FACS data was analyzed in FlowJo and the values corresponding to the percentage of MUC1 positive cells among viable human epithelial cells from each animal were exported to Excell and plotted as average for each treatment group. For each tumor isolate, frequencies of CAR-T cells (hCD45+) and tumor cells (hEpCAM+) among all viable human cells (Viability e780−, hHLA-ABC+) were plotted as both individual data points and as an average for each treatment group.

The results showed that CAR-T cells engineered with attributes (PD1/TRAC knockout and IL-12 release) were found in HCC70 tumors at much higher frequency than CAR-T cells without attributes.

These results suggest that addition of attributes enhances tumor infiltration and/or intratumoral CAR-T cell expansion. Enhanced tumor infiltration supports observed anti-tumor response, which was consistent with the results previously observed in FIG. 12.

Overall, these results suggest that addition of attributes are significant for achieving complete response in treatment of solid tumors.

Tumors from mice treated with CLS MUC1-A or CLS MUC1-C engineered CART cells were isolated from mice on day 54 (FIG. 18A), were homogenized in Accutase Cell Dissociation Reagent and were transferred to an incubator set at 37° C., 5% CO2 for 30 minutes. Dissociated cells were washed with 20 mL of ice-cold PBS containing 3% FBS and 30 passed through 100 um mesh. Tumor isolates were counted on vi-cell and stained for FACS analysis. In order to examine CAR-T cell infiltration in tumors, viable cells were identified using fixable viability dye e780, and human CAR T-cells were identified with human anti-CD45 antibody and a mouse anti-CD45 antibody. Both MUC-1A and MUC1-C engineered CAR-T cells could be detected in the tumor 54 days post challenge (FIG. 18D) with mean value of more than 40% for 10e6 CLS MUC1-A engineered CAR-T cells injected.

In a hostile tumor microenvironment CAR-T cells expansion, infiltration and effector functions can be inhibited by myriad of intratumoral factors. Therefore, even the most active CARs may hardly achieve clinical anti-tumor response without additional features. Here we demonstrate that some of the tumor microenvironment barriers can be successfully eliminated through combinations of attributes such as PD1 knockout, which limits CAR-T cells exhaustion, coupled with inducible IL-12 release, enhancing T-cell potency. The attributes included in this invention can synergize strategies to increase potency in developing successful lines of CAR-T cells against solid tumors.

It also appears from the above results that engineering MUC1 CAR T-cells is a promising approach that enhances potency and improve allogenic persistence in the context of solid tumors, especially to target Triple Negative Breast Cancer.

1-77. (canceled)

78. A method for manufacturing a population of engineered therapeutic immune cells, comprising the steps of:
a) providing immune cells originating from a patient or donor;
b) expressing in said cells an anti-MUC1 chimeric antigen receptor (CAR);
c) introducing at least one genetic modification(s) in the genome of said cell, said modification(s) being selected from those leading to an enhanced IL-2, IL-12, IL-15 or IL-18 expression;
d) expanding said cells to form a population of therapeutically effective immune cells.

79. The method of claim 78, wherein further genetic modification(s) is introduced into the genome leading to:
reduced or inactivated TCR expression;
reduced or inactivated B2M;
reduced interaction between TGFβ and TGFβR2; and/or
reduced interaction between PD1 and PDL1;

80. The method of claim 79, wherein said further genetic modification leads to the inactivation of the expression of at least one TGFbeta receptor.

81. The method of claim 78, wherein said genetic modification(s) is (are) obtained by using sequence specific gene editing reagents.

82. An engineered immune cell expressing an anti-MUC1 CAR obtainable by the method of claim 78.

83. An engineered immune cell expressing an anti-MUC1 CAR, wherein said anti-MUC1 CAR comprises at least:
an extracellular ligand binding-domain comprising VH and VL from a monoclonal antibody targeting tMUC1 epitope(s);
a transmembrane domain; and
a cytoplasmic domain comprising a CD3 zeta signalling domain and a co-stimulatory domain,
wherein said extra cellular ligand binding-domain is directed against an antigen of the MUC1 polypeptide region HGVTSAPDTRPAPGSTAPPA (SEQ ID NO:1), and
wherein said CAR is co-expressed with another exogenous sequence encoding a cytokine selected from IL-2, IL-12, IL-15 and IL-18.

84. The engineered immune cell of claim 83, wherein said engineered immune cell comprises at least one further genetic modification introduced into the genome leading to:
reduced or inactivated TCR expression;
reduced or inactivated B2M;
reduced interaction between TGFβ and TGFβR2; and/or
reduced interaction between PD1 and PDL1.

85. The engineered immune cell of claim 83, wherein said anti-MUC1 comprises an extra cellular ligand binding-domain ScFvs having at least 95% sequence identity with respectively MUC1-A ScFv (SEQ ID NO:17), MUC1-B (SEQ ID NO:27), MUC1-C(SEQ ID NO:37), and/or MUC1-D (SEQ ID NO:47).

86. The engineered immune cell of claim 83, wherein:
said variable light (VL) chain comprises CDRs selected from SEQ ID NO:11 (CDR-VL1-A), SEQ ID NO:12 (CDR-VL2-A) and SEQ ID NO:13 (CDR-VL3-A), and
said variable heavy (VH) chain comprises CDRs selected from SEQ ID NO:14 (CDR-VH1-A), SEQ ID NO:15 (CDR-VH2-A) and SEQ ID NO:16 (CDR-VH3-A).

87. The engineered immune cell of claim 83, wherein said anti-MUC1 has at least 80% overall amino acid sequence identity with SEQ ID NO:18 (CLS MUC1-A CAR).

88. The engineered immune cell of claim 83, wherein:
said variable light (VL) chain comprises CDRs selected from SEQ ID NO:21 (CDR-VL1-B), SEQ ID NO:22 (CDR-VL2-B) and SEQ ID NO:23 (CDR-VL3-B), and
said variable heavy (VH) chain comprises CDRs selected from SEQ ID NO:24 (CDR-VH1-B), SEQ ID NO:25 (CDR-VH2-B) and SEQ ID NO:26 (CDR-VH3-B).

89. The engineered immune cell of claim 83, wherein said anti-MUC1 has at least 80% overall amino acid sequence identity with SEQ ID NO:28 (CLS MUC1-B CAR).

90. The engineered immune cell of claim 83, wherein:
said variable light (VL) chain comprises CDRs selected from SEQ ID NO: 31(CDR-VL1-C), SEQ ID NO:32 (CDR-VL2-C) and SEQ ID NO:33 (CDR-VL3-C), and
said variable heavy (VH) chain comprises CDRs selected from SEQ ID NO:34 (CDR-VH1-C), SEQ ID NO:35 (CDR-VH2-C) and SEQ ID NO:36 (CDR-VH3-C).

91. The engineered immune cell of claim 83, wherein said CAR has at least 80% overall amino acid sequence identity with SEQ ID NO:38 (CLS MUC1-C CAR).

92. The engineered immune cell of claim 83, wherein:
said variable light (VL) chain comprising CDRs selected from SEQ ID NO:41 (CDR-VL1-D), SEQ ID NO:42 (CDR-VL2-D) and SEQ ID NO:43 (CDR-VL3-D), and
said variable heavy (VH) chain comprising CDRs selected from SEQ ID NO:44 (CDR-VH1-D), SEQ ID NO:45 (CDR-VH2-D) and SEQ ID NO:46 (CDR-VH3-D).

93. The engineered immune cell of claim 83, wherein said CAR has at least 80% overall amino acid sequence identity with SEQ ID NO:48 (MUC1-D CAR).

94. The engineered immune cell of claim 83, wherein said immune cell is T-cell or NK cell.

95. A population of cells comprising more than 25% of engineered immune cells of claim 83.

96. A therapeutic composition comprising the population of cells of claim 95.

97. A method comprising administering the population of cells of claim 95 to a cancer patient.
